(19) United States
(12) Patent Application Publication
(10) Pub. No.: US 2008/0280887 A1
(43) Pub. Date: Nov. 13, 2008

(54) Selected Cgrp-Antagonists, Processes for Preparing Them and Their Use as Pharmaceutical Compositions

(76) Inventors: Stephan Georg Mueller, Warthausen (DE); Klaus Rudolf, Warthausen (DE); Philipp Lustenberger, Basel (CH); Gerhard Schaenzle, Biberach (DE); Dirk Stenkamp, Biberach (DE); Henri Doods, Warthausen (DE); Kirsten Arndt, Biberach (DE)

Correspondence Address: MICHAEL P. MORRIS;BOEHRINGER INGELHEIM USA CORPORATION, 900 RIDGEBURY ROAD, P. O. BOX 368, RIDGEFIELD, CT, 06877-0368, US

(21) Appl. No.: 12/067,885

(22) PCT Filed: Sep. 27, 2006
(86) PCT No.: PCT/EP06/66789

§ 371 Date: Jun. 9, 2008

(30) Foreign Application Priority Data
Sep. 28, 2005 (EP) 05021282.8

(51) Int. Cl. A61K 31/496; A61P 25/06; A61P 3/10; C07D 401/14; C07D 413/14; A61K 31/551; A61K 31/5377; A61K 31/519

(52) U.S. Cl. 514/221; 544/286; 514/253.02; 544/364; 544/121; 540/500; 514/235.8; 514/252.16; 514/253.09; 514/253.13

(57) ABSTRACT

The invention relates to CGRP-antagonists of general formula (I), wherein R¹, R², R³, R⁴ and R⁵ are defined in claim 1, to the tautomers, isomers, diastereomers, enantiomers, hydrates, mixtures salts and salt hydrates thereof, in particular to 10 salts thereof, which are physiologically compatible with acids or inorganic or organic bases and to compounds of general formula (I), wherein one or several hydrogen atoms are substituted by deuterium. Drugs containing said compounds, the use thereof and a method for the production thereof are also disclosed.

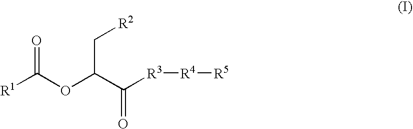

The present invention relates to CGRP antagonists of general formula I

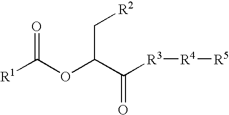

wherein R¹, R², R³, R⁴ and R⁵ are defined as in claim 1, the tautomers, the isomers, the diastereomers, the enantiomers, the hydrates thereof, the mixtures thereof and the salts thereof and the hydrates of the salts thereof, particularly the physiologically acceptable salts thereof with inorganic or organic acids or bases, as well as those compounds of general formula I wherein one or more hydrogen atoms are replaced by deuterium, pharmaceutical compositions containing these compounds, their use and processes for preparing them.

### PRIOR ART

CGRP antagonists for the treatment of migraine have already been described in International Patent Applications PCT/EP97/04862 and PCT/EP04/000087.

### DETAILED DESCRIPTION OF THE INVENTION

In the above general formula I in a first embodiment

R¹ denotes a group selected from

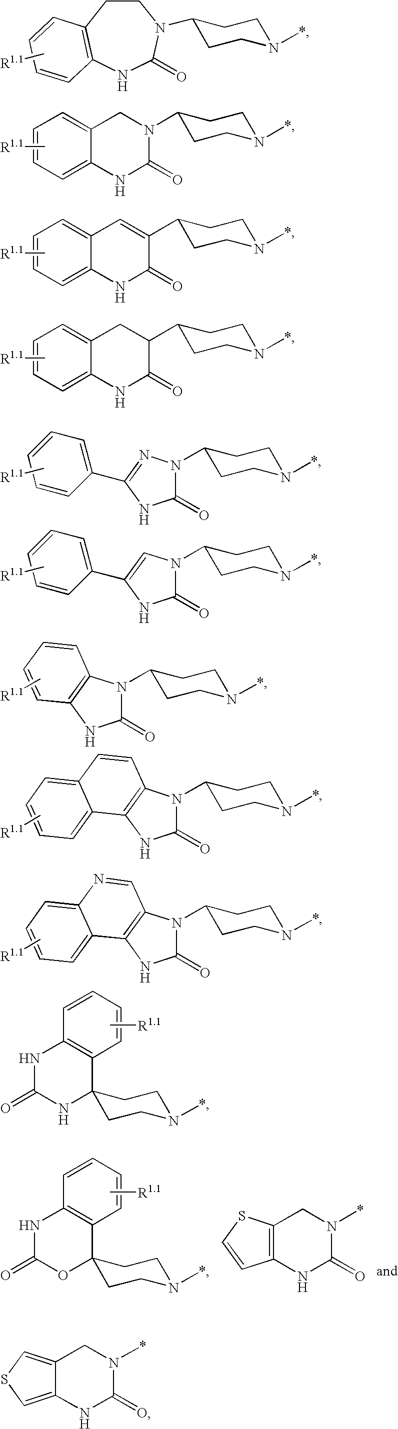

wherein
R^{1.1} denotes H, halogen, HO, F₃C or C₁₋₆-alkyl-O—,
   R² denotes a group of general formulae II

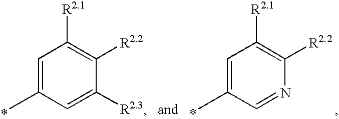

wherein
R^{2.1} denotes H, halogen, C₁₋₃-alkyl-O, C₁₋₃-alkyl or F₃C,
R^{2.2} denotes H, H₂N, HO, H₃C—O, H—C(O)—O or C₁₋₃-alkyl-C(O)—O,
R^{2.3} denotes H, halogen, C₁₋₃-alkyl or F₃C—,
   R³ denotes a group of general formulae III

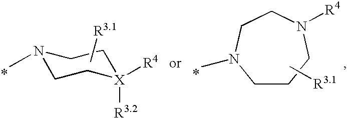

wherein
X denotes N or C,
R^{3.1} denotes H, C₁₋₃-alkyl or R^{3.1.1}—(O)C,
R^{3.1.1} denotes HO or C₁₋₆-alkyl-O,
R^{3.2} denotes a pair of free electrons, if X═N, or
R^{3.2} denotes H or C₁₋₃-alkyl-, if X═C,
   R⁴ denotes a group selected from

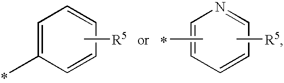

R⁵ denotes R^{5.1}—O—C(O)— and
R^{5.1} denotes H, C₁₋₈-alkyl, phenyl, indanyl, pyridyl-C₁₋₃-alkylene, HO—C₂₋₄-alkylene, C₁₋₆-alkyl-O—C₂₋₄-alkylene, HO—C₂₋₄-alkylene-O—C₂₋₄-alkylene, C₁₋₆-alkyl-C₂₋₄-alkylene-O—C₂₋₄-alkylene, H₂N—C₂₋₄-alkylene, (C₁₋₆-alkyl)-NH—C₂₋₄-alkylene, (C₁₋₆-alkyl)₂N—C₂₋₄-alkylene, H₂N—C(O)—C₁₋₃-alkylene, (C₁₋₆-alkyl)-NH—C(O)—C₁₋₃-alkylene, (C₁₋₆-alkyl)₂N—C(O)—C₁₋₃-alkylene, C₁₋₆-alkyl-C(O)—O—C₁₋₃-alkylene, C₁₋₆-alkyl-O—C(O)—O—C₁₋₃-alkylene, R^{5.1.1}—C(O)—C₁₋₃-alkylene or R^{5.1.2}—C₂₋₄-alkylene,
R^{5.1.1} denotes a group selected from

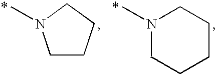

R^{5.1.2} denotes a group selected from

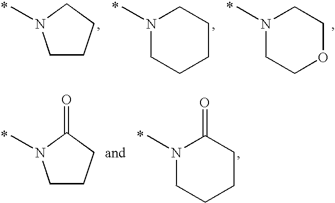

the tautomers, the diastereomers, the enantiomers, the hydrates, the mixtures thereof and the salts thereof and the hydrates of the salts, particularly the physiologically acceptable salts thereof with inorganic or organic acids or bases.

A second embodiment of the present invention comprises those compounds of the above general formula I, wherein

R¹ denotes a group selected from

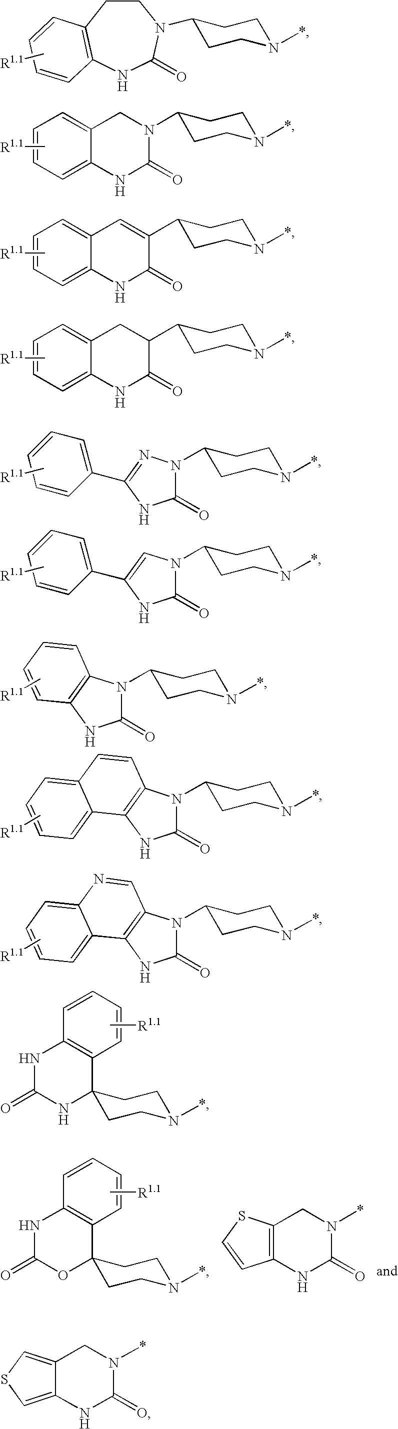

wherein
R^{1.1} denotes H or H₃C—O—,
   R² denotes a group selected from

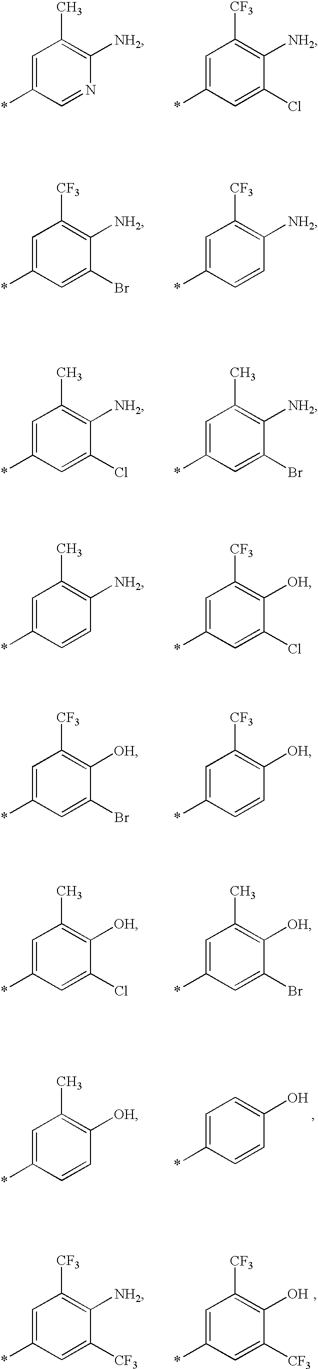

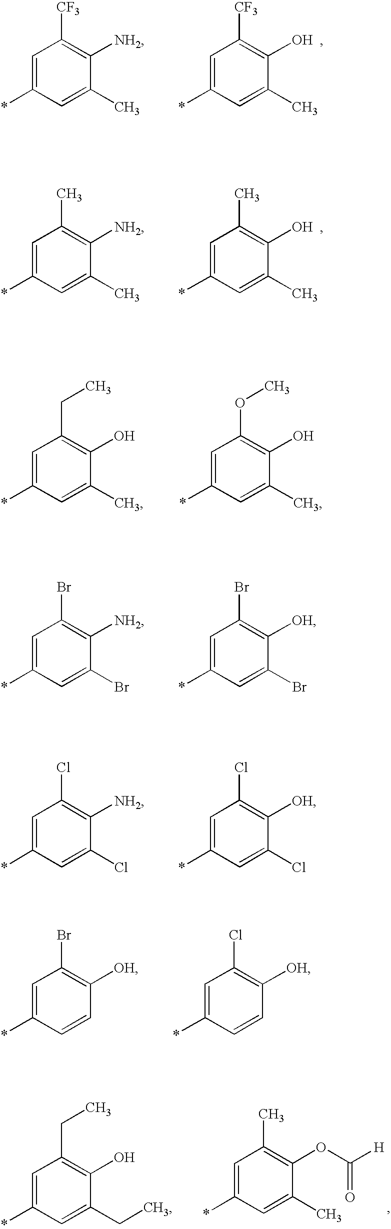

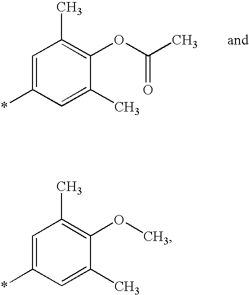

R³-R⁴ together denote a group selected from

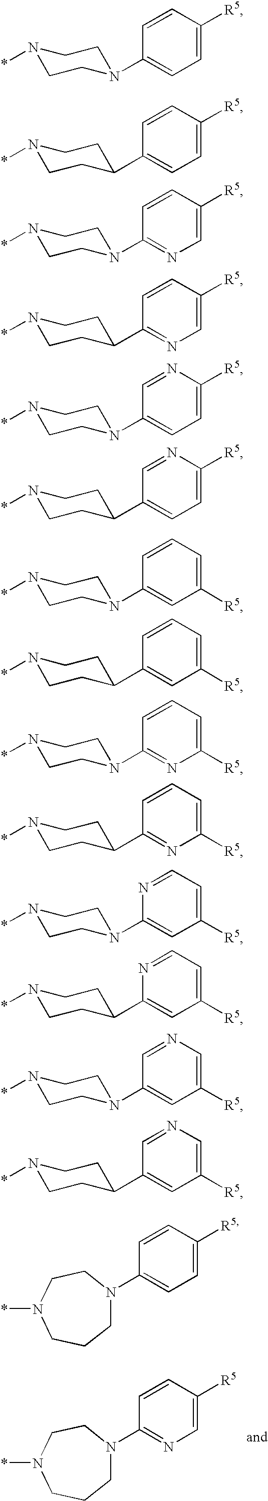

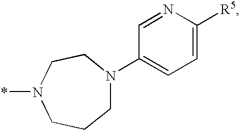

R⁵ denotes R^{5.1}—O—C(O)— and
R^{5.1} denotes H, C₁₋₈-alkyl, phenyl, indanyl, pyridyl-C₁₋₃-alkylene, HO—C₂₋₄-alkylene, C₁₋₆-alkyl-O—C₂₋₄-alkylene, HO—C₂₋₄-alkylene-O—C₂₋₄-alkylene, C₁₋₆-alkyl-C₂₋₄-alkylene-O—C₂₋₄-alkylene, H₂N—C₂₋₄-alkylene, (C₁₋₆-alkyl)-NH—C₂₋₄-alkylene, (C₁₋₆-alkyl)₂N—C₂₋₄-alkylene, H₂N—C(O)—C₁₋₃-alkylene, (C₁₋₆-alkyl)-NH—C(O)—C₁₋₃-alkylene, (C₁₋₆-alkyl)₂N—C(O)—C₁₋₃-alkylene, C₁₋₆-alkyl-C(O)—O—C₁₋₃-alkylene, C₁₋₆-alkyl-O—C(O)—O—C₁₋₃-alkylene, R^{5.1.1}—C(O)—C₁₋₃-alkylene or R^{5.1.2}—C₂₋₄-alkylene,
R^{5.1.1} denotes a group selected from

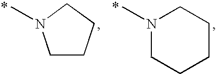

R^{5.1.2} denotes a group selected from

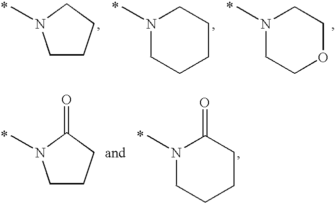

the tautomers, the diastereomers, the enantiomers, the hydrates, the mixtures thereof and the salts thereof and the hydrates of the salts, particularly the physiologically acceptable salts thereof with inorganic or organic acids or bases.

A third embodiment of the present invention comprises those compounds of the above general formula I, wherein

R¹ denotes a group selected from

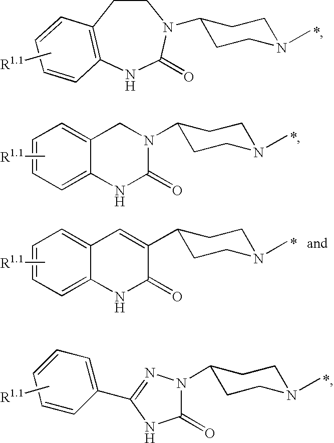

R² denotes a group selected from

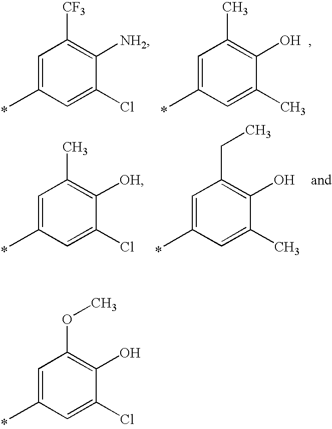

R³-R⁴ together denote a group selected from

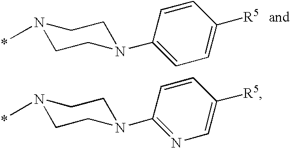

R⁵ denotes R^{5.1}—O—C(O)— and
R^{5.1} denotes H, C₁₋₆-alkyl, phenyl, indanyl, pyridyl-CH₂, C₁₋₃-alkyl-O—C₂₋₄-alkylene, C₁₋₃-alkyl-O—C₂₋₄-alkylene-O—C₂₋₄-alkylene, (C₁₋₃-alkyl)₂N—C₂₋₄-alkylene, (C₁₋₃-alkyl)₂N—C(O)—C₁₋₃-alkylene, C₁₋₆-alkyl-C(O)—O—C₁₋₃-alkylene, C₁₋₃-alkyl-O—C(O)—O—C₁₋₃-alkylene,
R^{5.1.1}—C(O)—C₁₋₃-alkylene or R^{5.1.2}—C₂₋₄-alkylene,
R^{5.1.1} denotes a group selected from

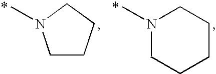

R^{5.1.2} denotes a group selected from

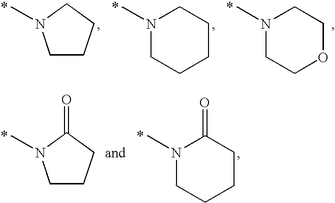

the tautomers, the diastereomers, the enantiomers, the hydrates, the mixtures thereof and the salts thereof and the hydrates of the salts, particularly the physiologically acceptable salts thereof with inorganic or organic acids or bases.

The following compounds are also mentioned as examples of most particularly preferred compounds of the above general formula I:

| No. | Structure |
|---|---|
| (1) | 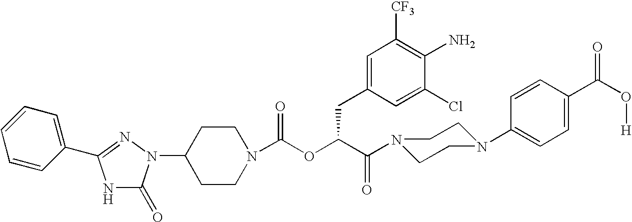 |
| (2) | 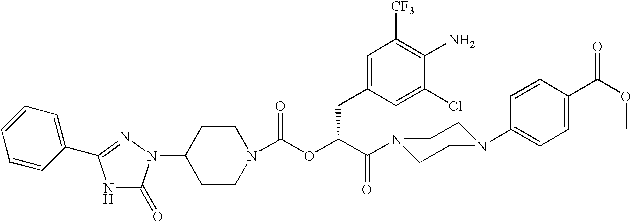 |
| (3) | 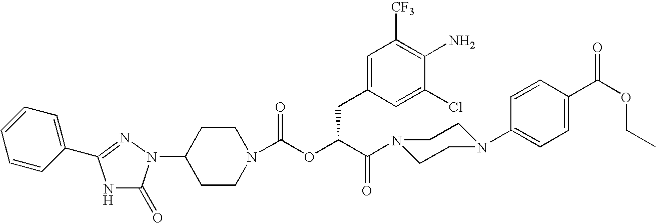 |
| (4) | 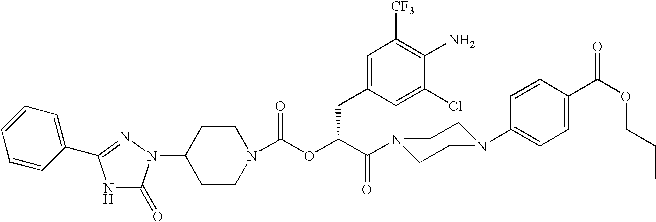 |
| (5) | 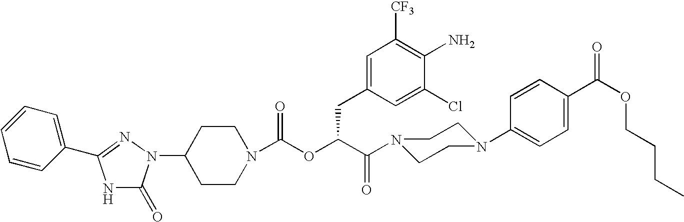 |
| (6) | 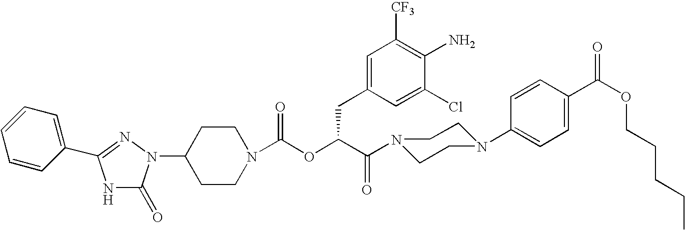 |
| (7) | 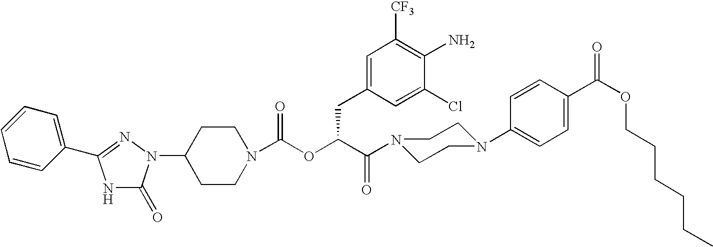 |
| (8) | 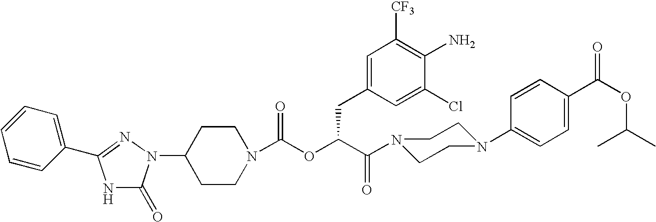 |
| (9) | 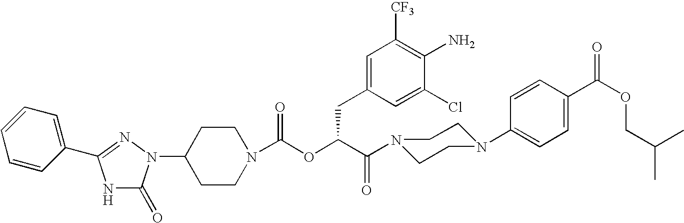 |
| (10) | 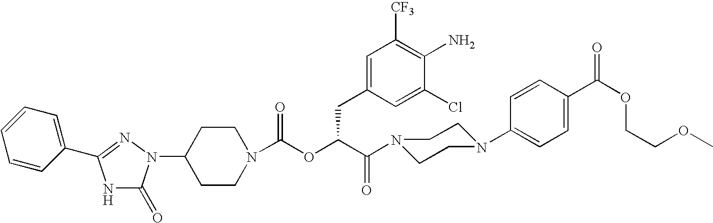 |
| (11) | 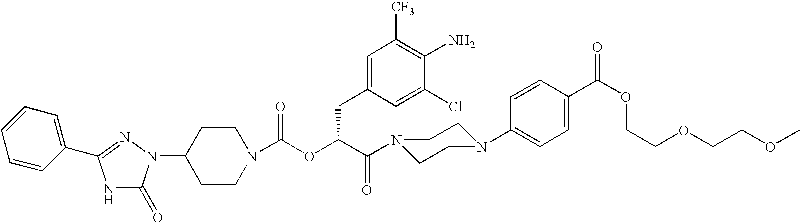 |
| (12) | 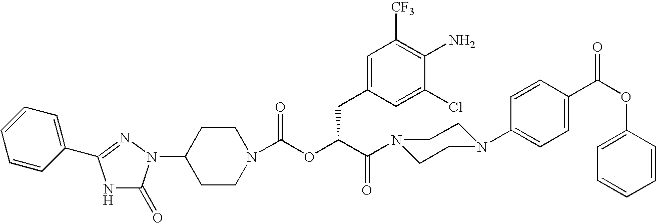 |
| (13) | 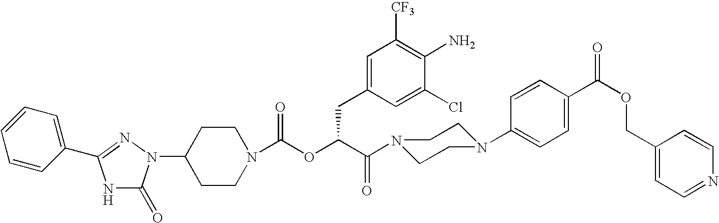 |
| (14) | 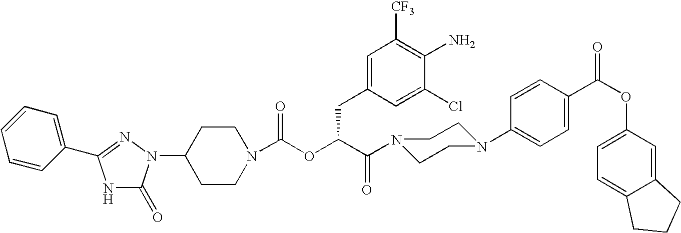 |
| (15) | 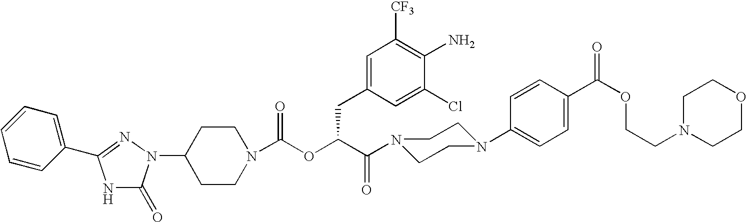 |
| (16) | 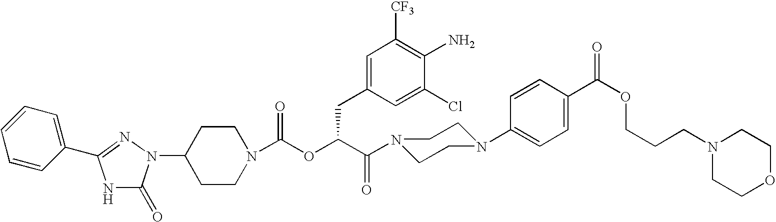 |
| (17) | 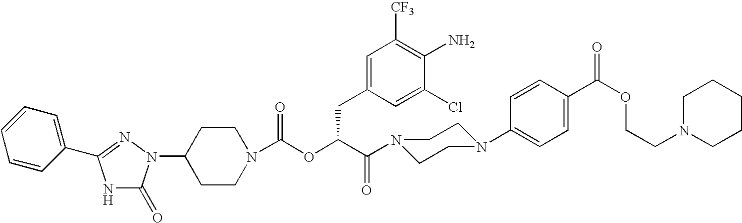 |
| (18) | 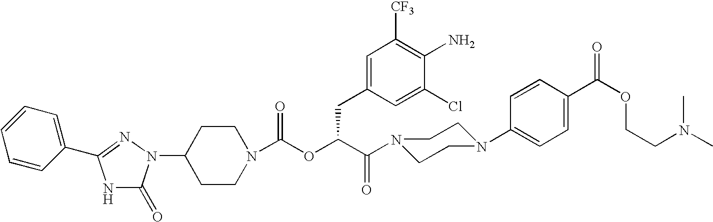 |
| (19) | 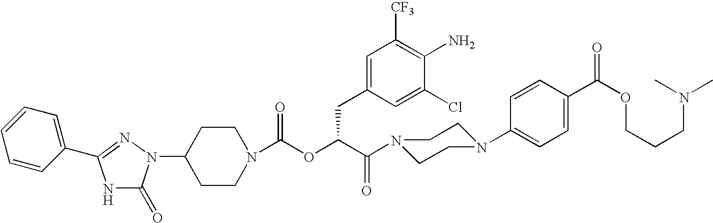 |
| (20) | 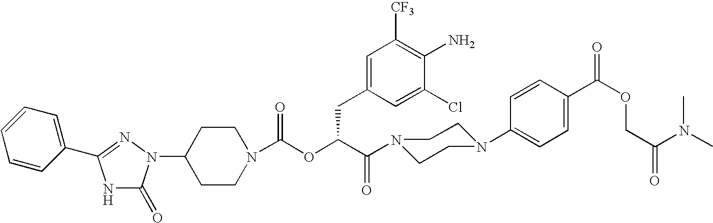 |
| (21) | 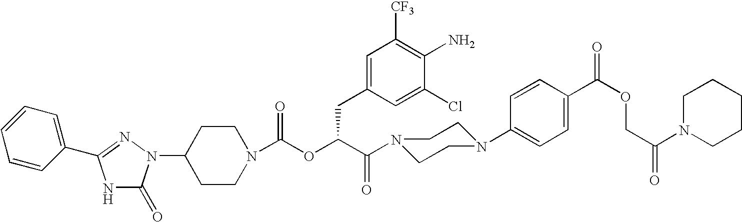 |
| (22) | 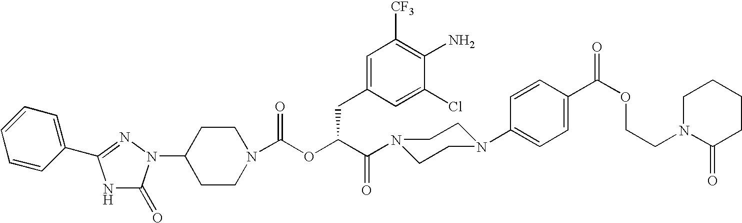 |
| (23) | 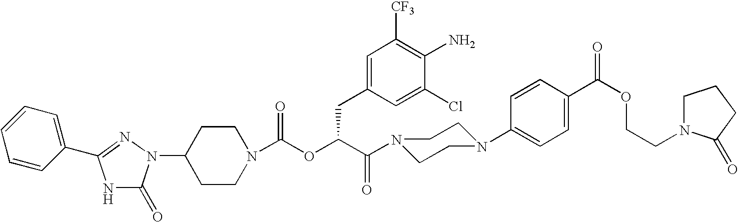 |
| (24) | 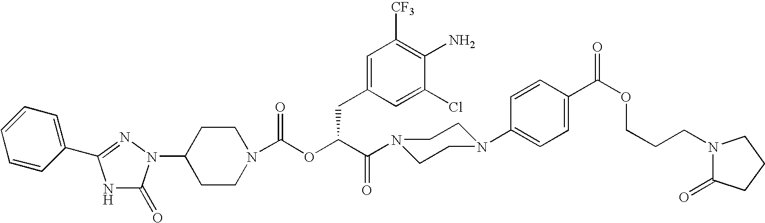 |
| (25) | 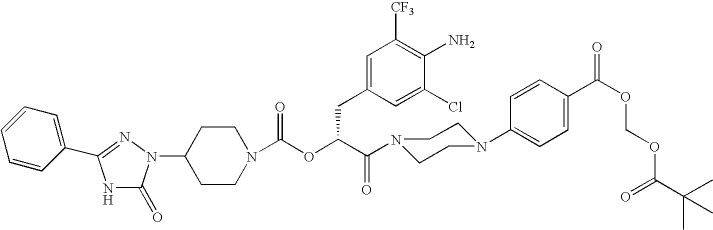 |
| (26) | 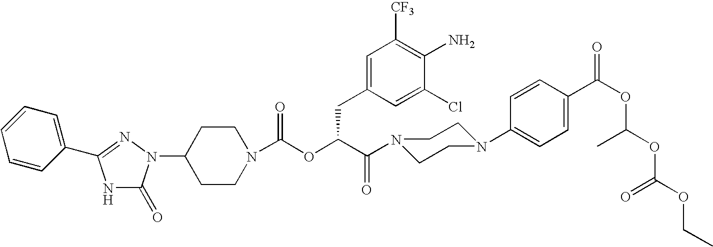 |
| (27) | 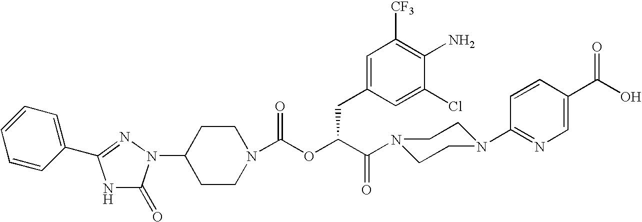 |
| (28) | 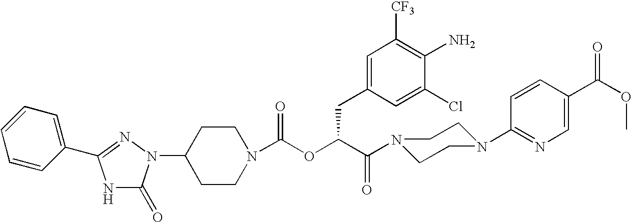 |
| (29) | 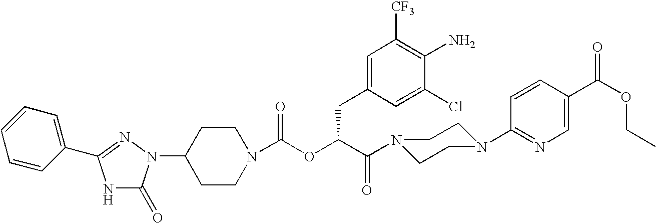 |
| (30) | 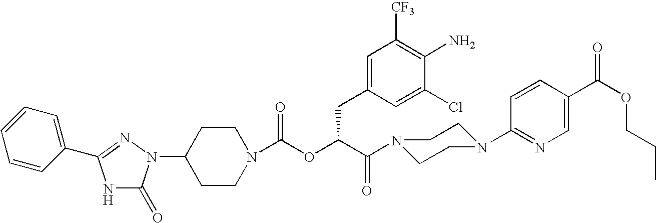 |
| (31) | 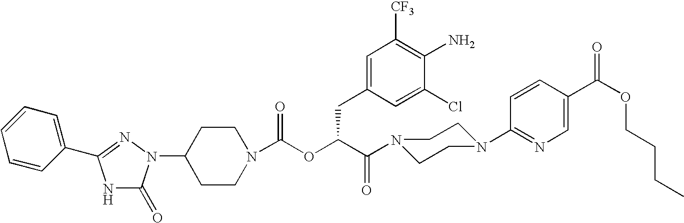 |
| (32) | 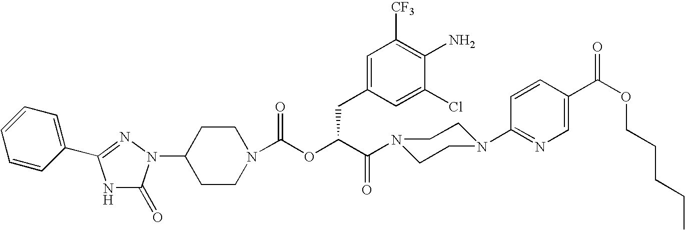 |
| (33) | 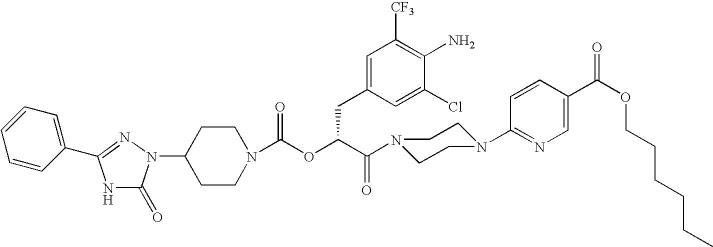 |
| (34) | 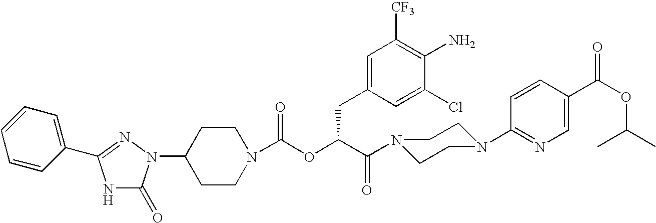 |
| (35) | 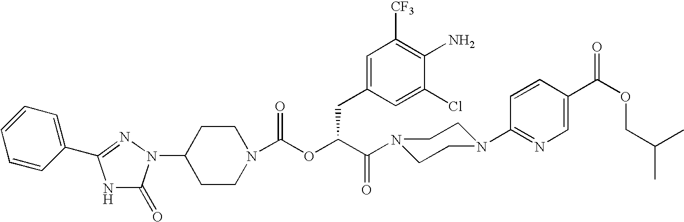 |
| (36) | 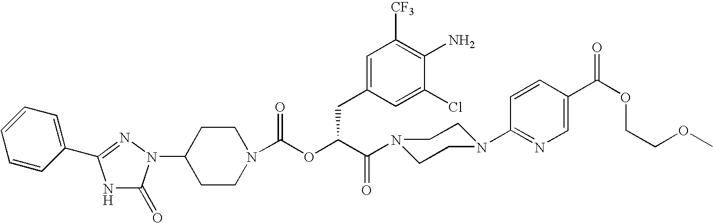 |
| (37) | 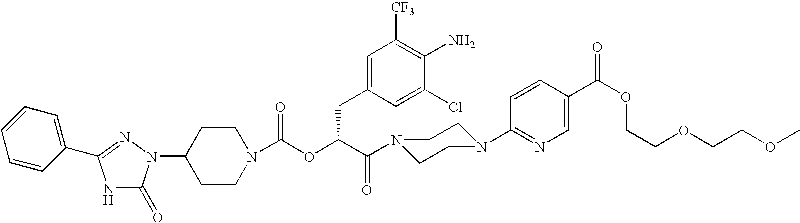 |
| (38) | 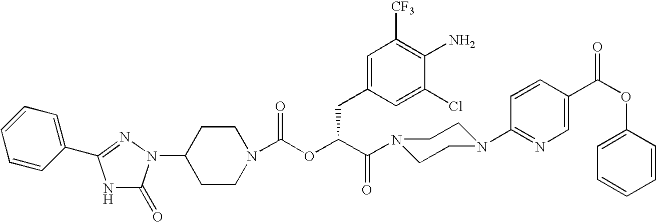 |
| (39) | 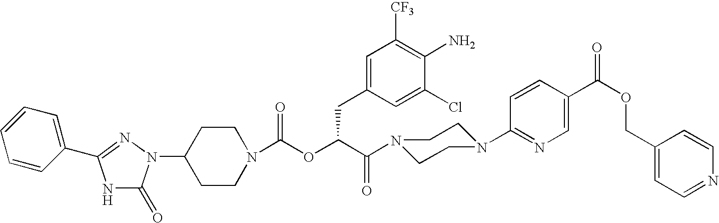 |
| (40) | 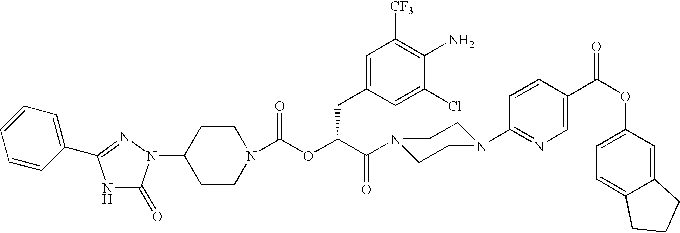 |
| (41) | 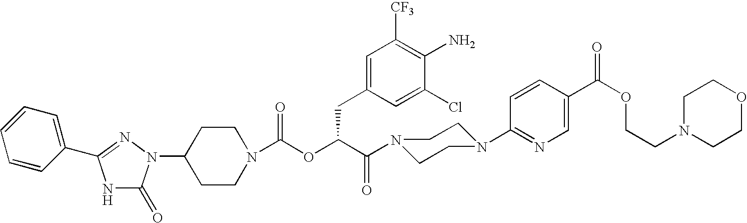 |
| (42) | 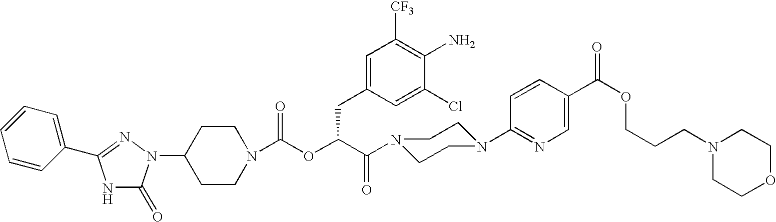 |
| (43) | 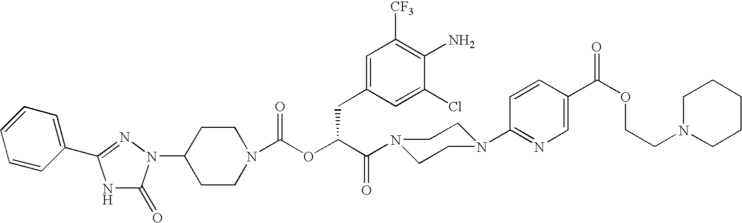 |
| (44) | 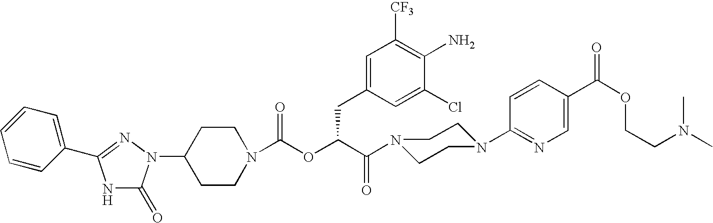 |
| (45) | 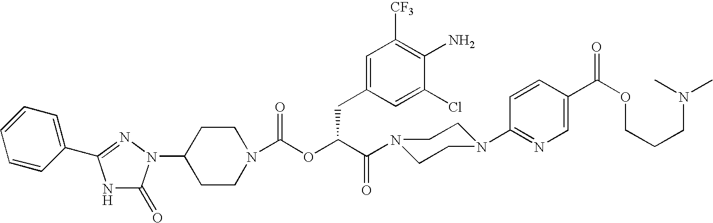 |
| (46) | 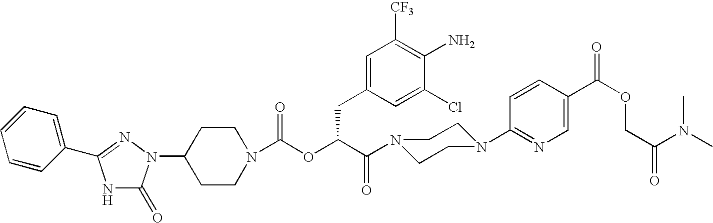 |
| (47) | 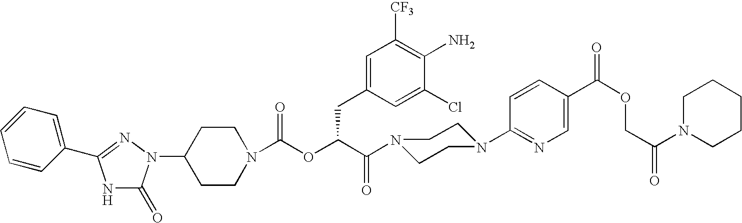 |
| (48) | 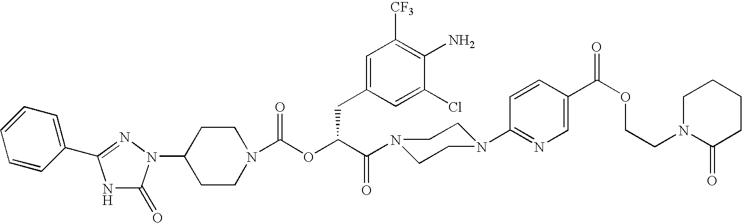 |
| (49) | 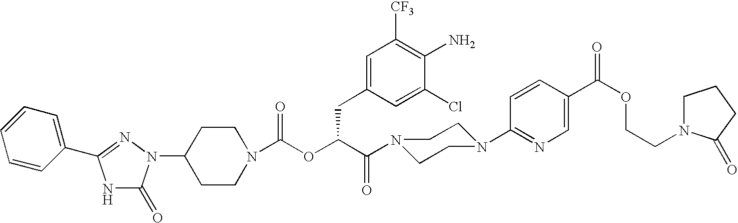 |
| (50) | 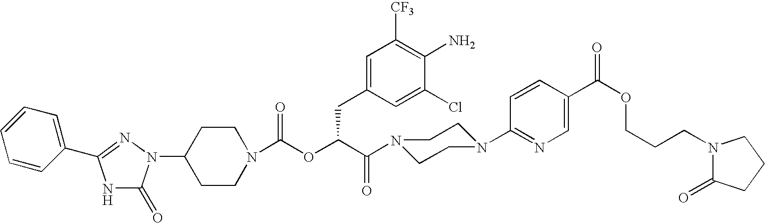 |
| (51) | 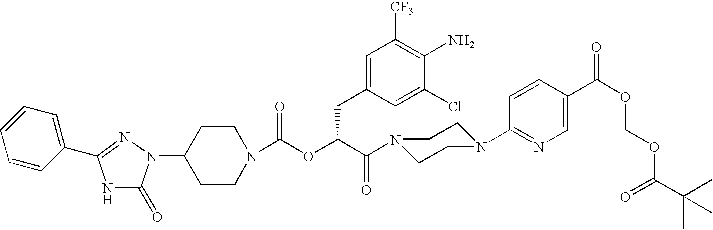 |
| (52) | 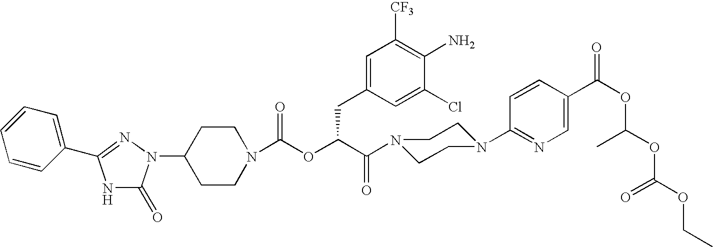 |
| (53) | 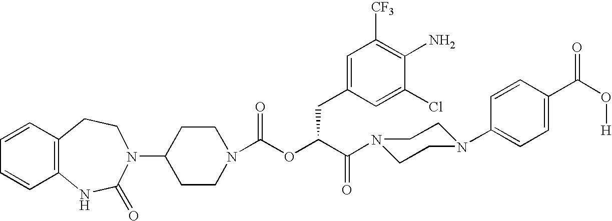 |
| (54) | 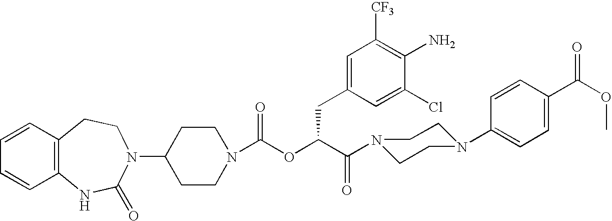 |
| (55) | 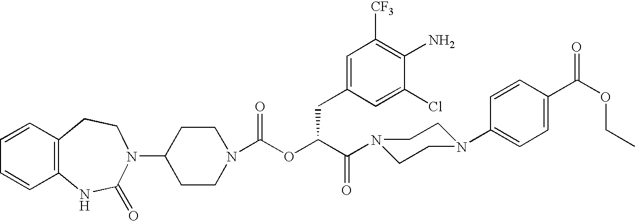 |
| (56) | 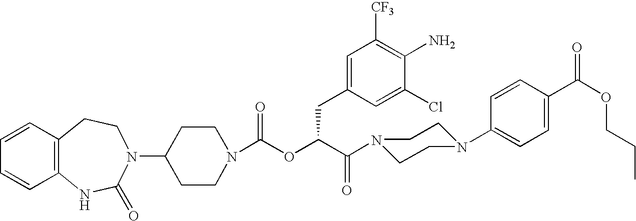 |
| (57) | 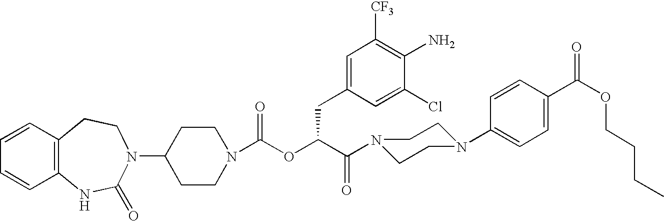 |
| (58) | 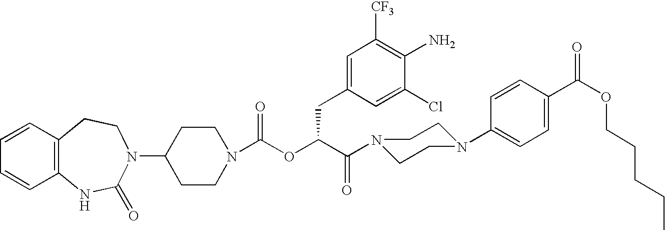 |
| (59) | 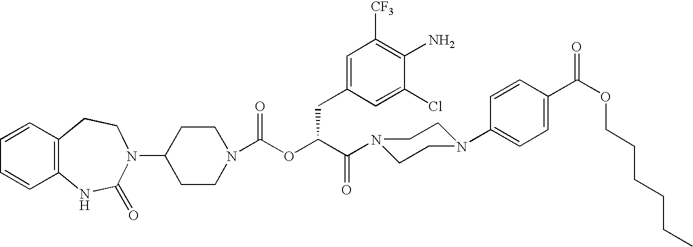 |
| (60) | 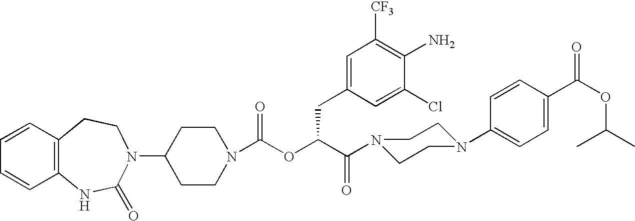 |
| (61) | 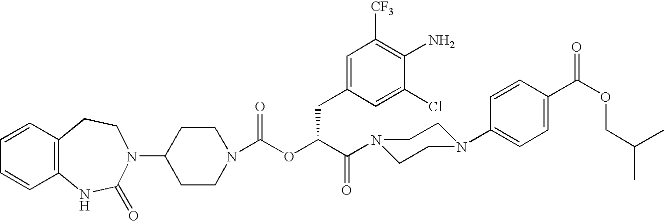 |
| (62) | 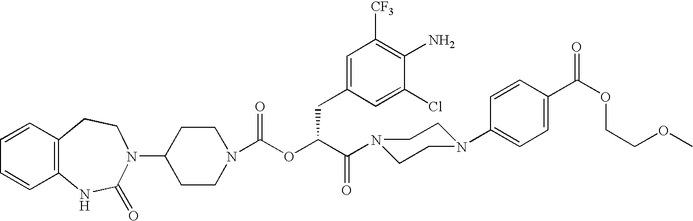 |
| (63) | 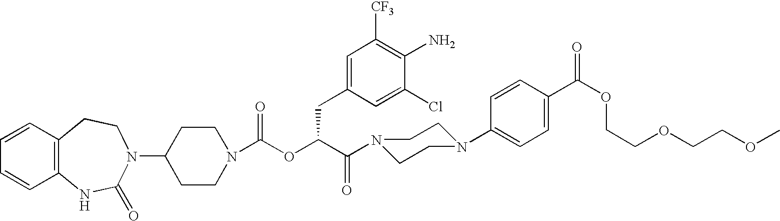 |
| (64) | 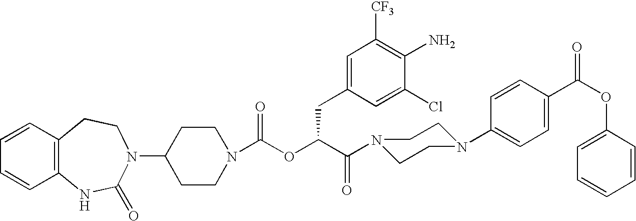 |
| (65) | 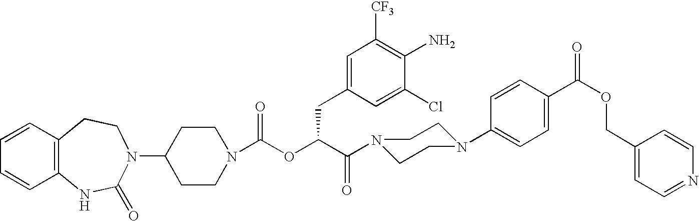 |
| (66) | 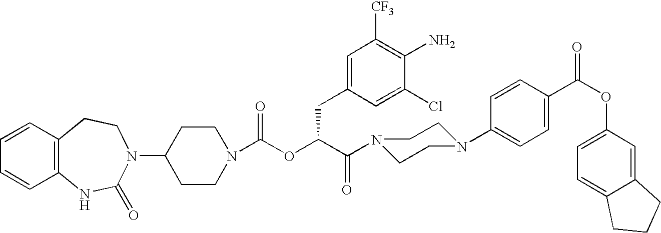 |
| (67) | 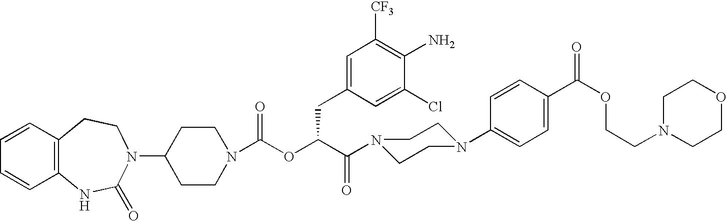 |
| (68) | 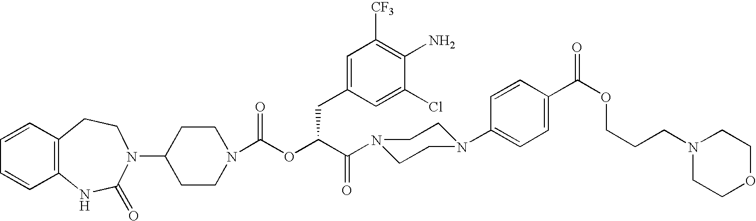 |
| (69) | 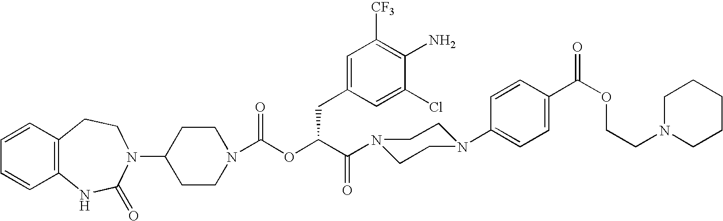 |
| (70) | 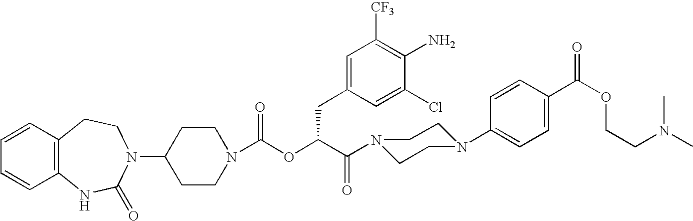 |
| (71) | 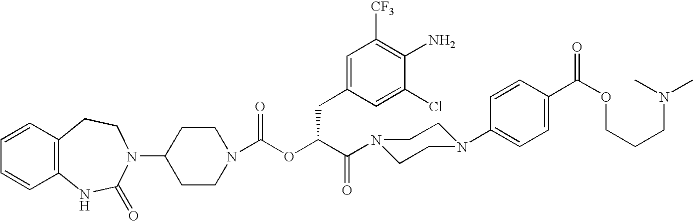 |
| (72) | 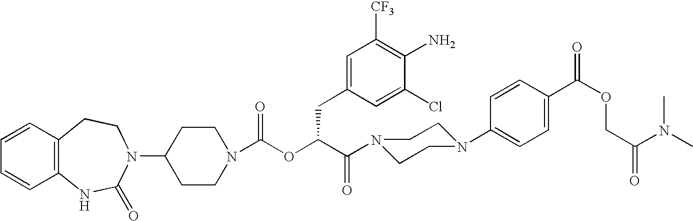 |
| (73) | 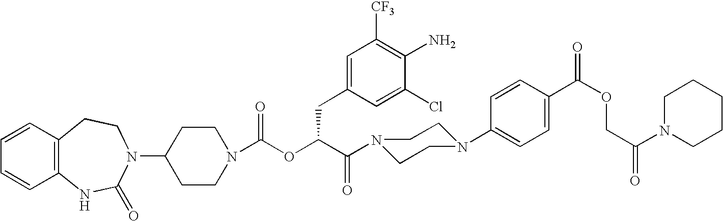 |
| (74) | 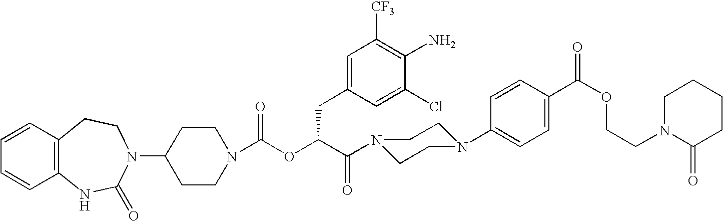 |
| (75) | 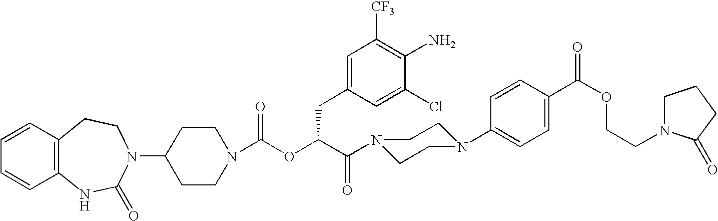 |
| (76) | 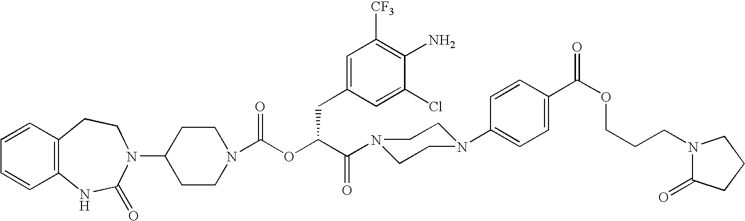 |
| (77) | 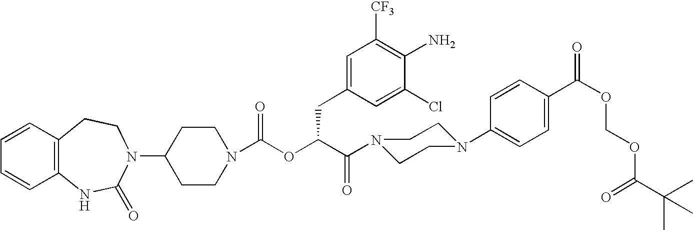 |
| (78) | 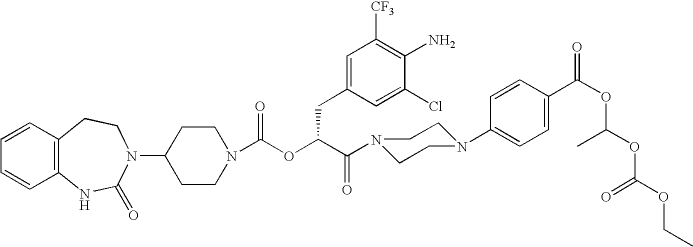 |
| (79) | 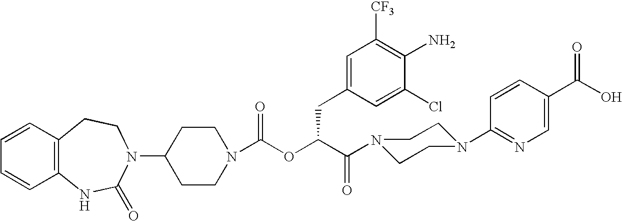 |
| (80) | 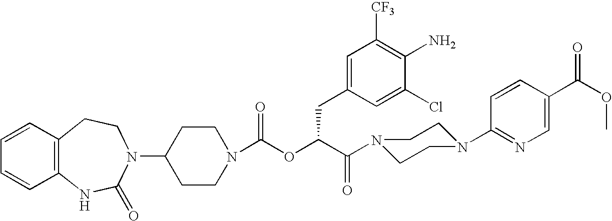 |
| (81) | 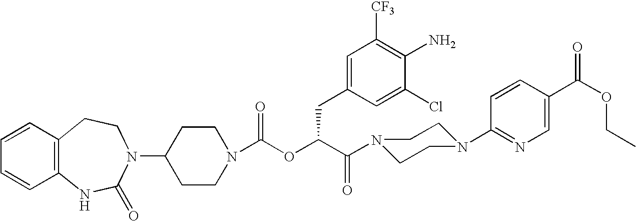 |
| (82) | 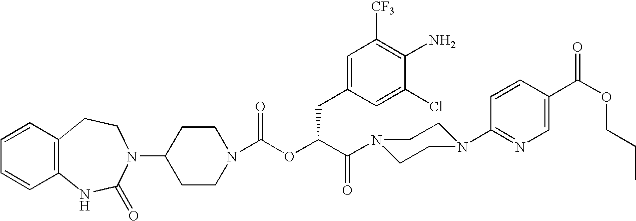 |
| (83) | 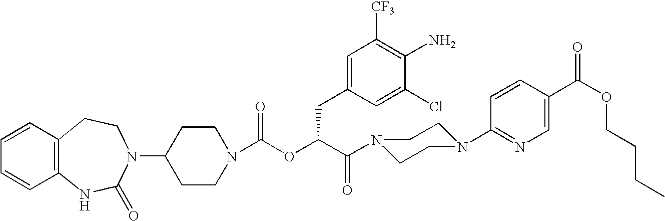 |
| (84) | 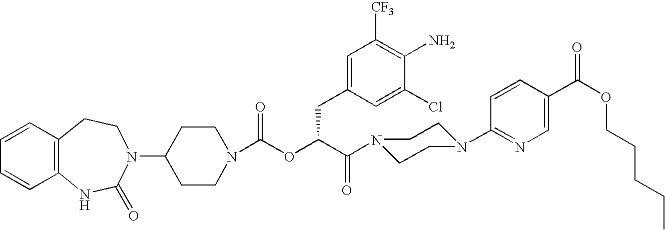 |
| (85) | 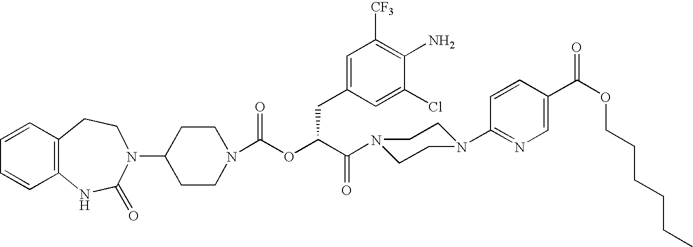 |
| (86) | 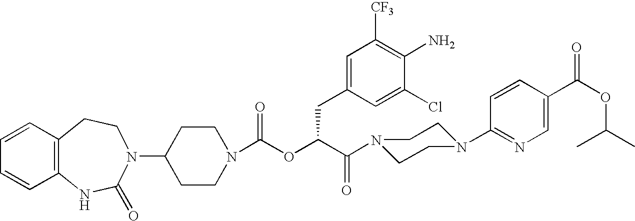 |
| (87) | 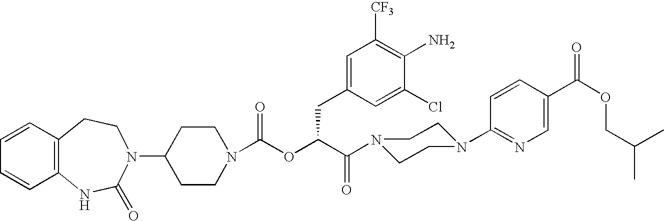 |
| (88) | 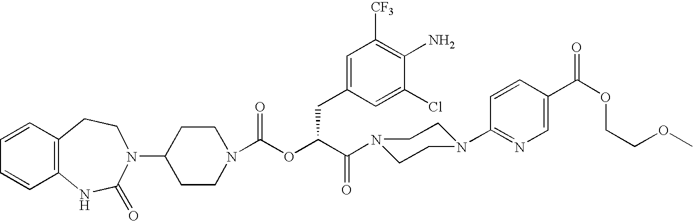 |
| (89) | 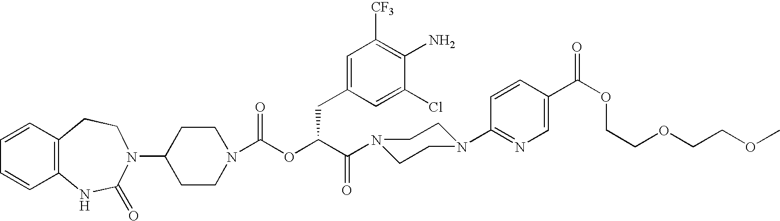 |
| (90) | 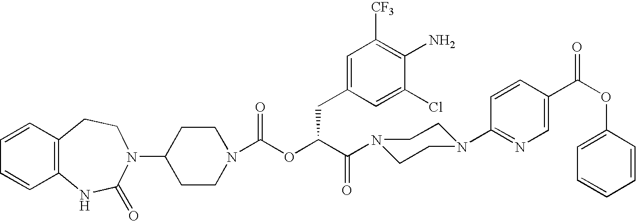 |
| (91) | 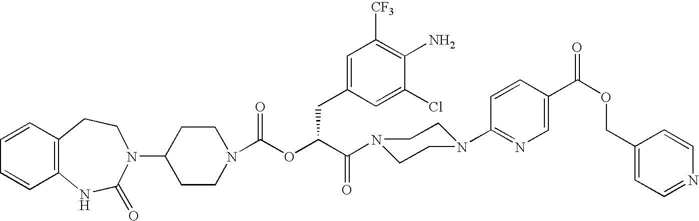 |
| (92) | 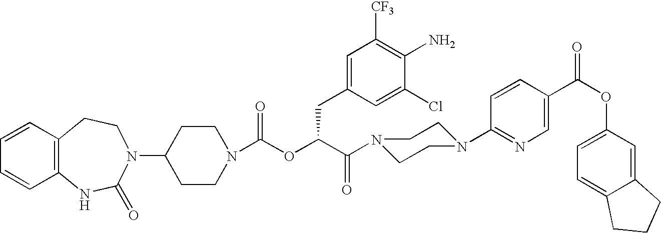 |
| (93) | 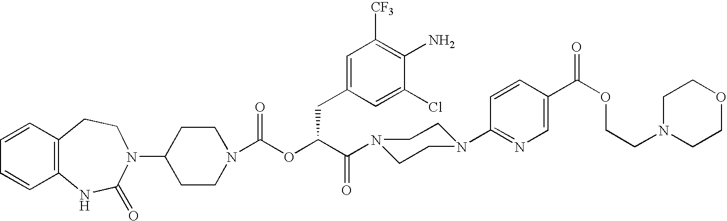 |
| (94) | 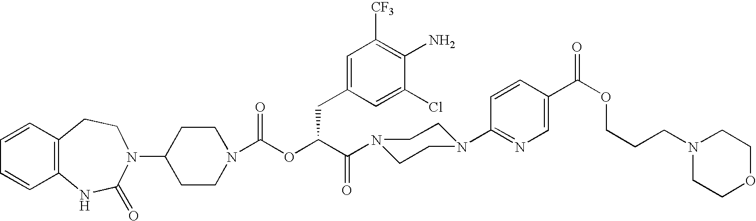 |
| (95) | 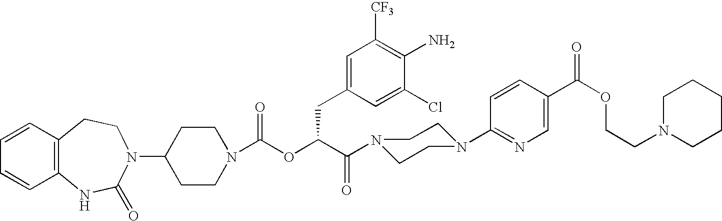 |
| (96) | 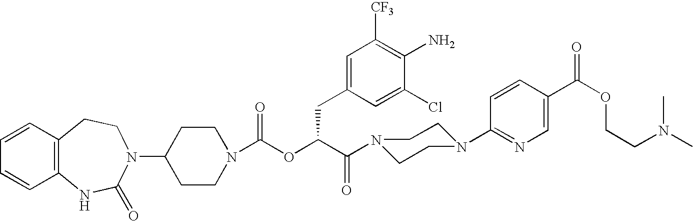 |
| (97) | 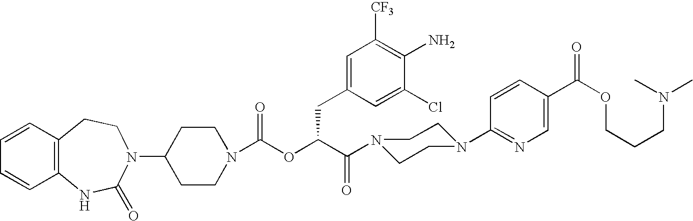 |
| (98) | 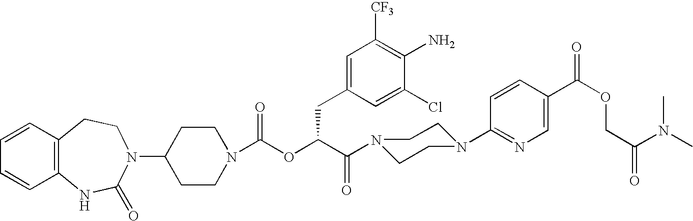 |
| (99) | 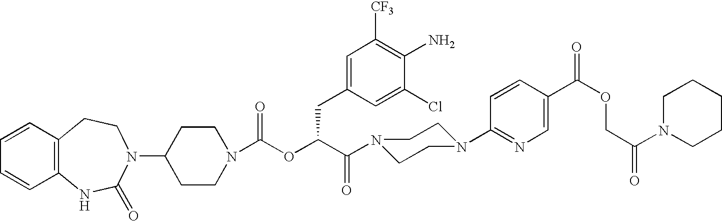 |
| (100) | 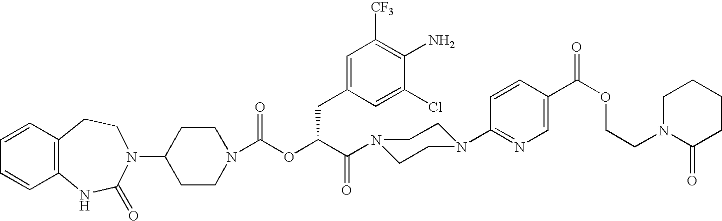 |
| (101) | 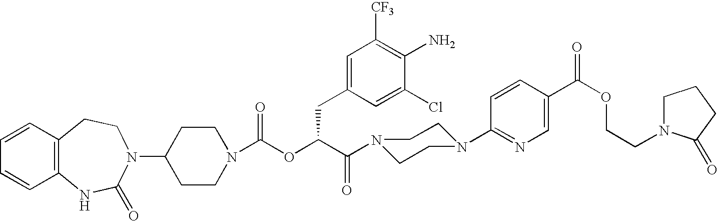 |
| (102) | 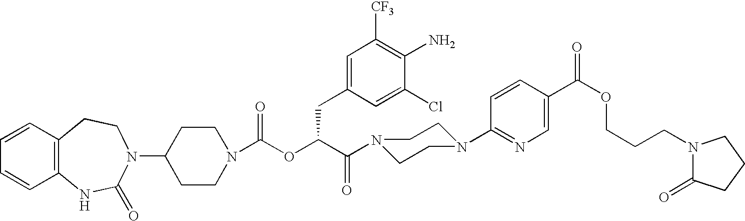 |
| (103) | 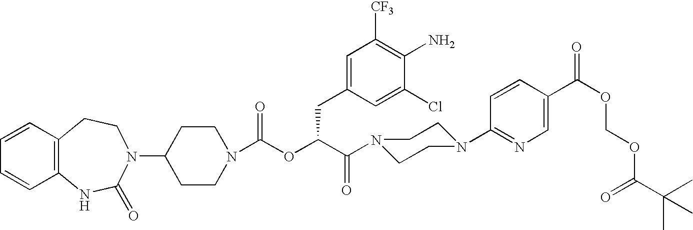 |
| (104) | 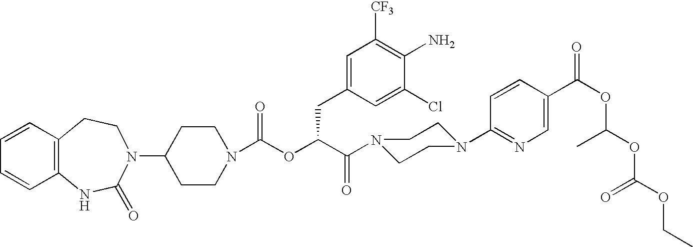 |
| (105) | 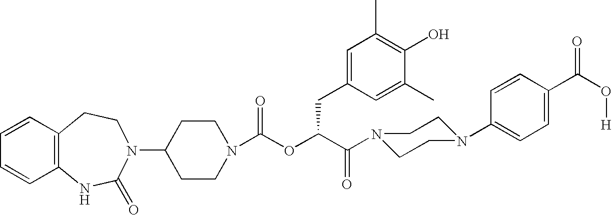 |
| (106) | 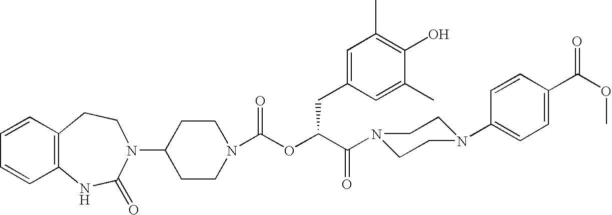 |
| (107) | 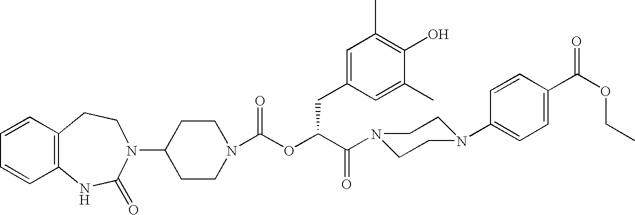 |
| (108) | 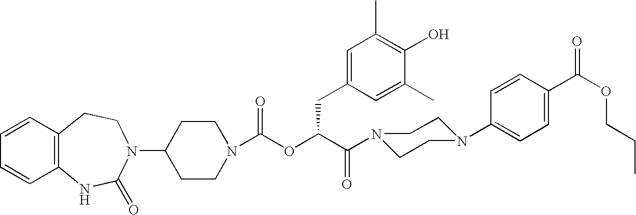 |
| (109) | 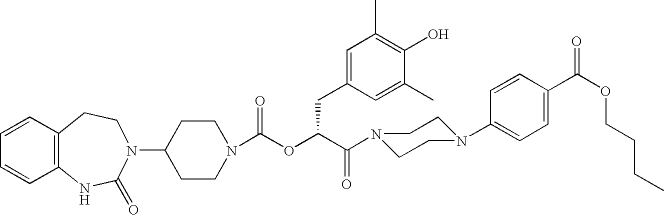 |
| (110) | 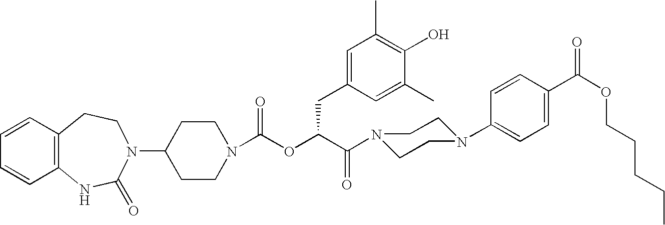 |
| (111) | 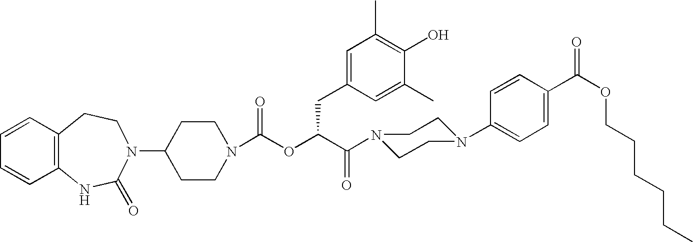 |
| (112) | 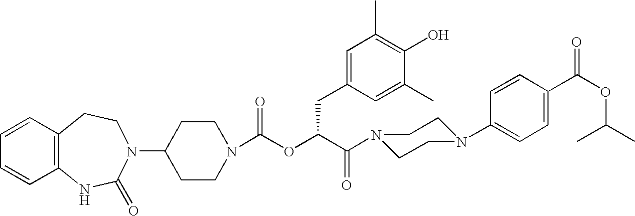 |
| (113) | 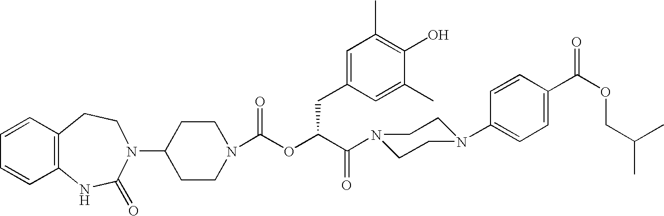 |
| (114) | 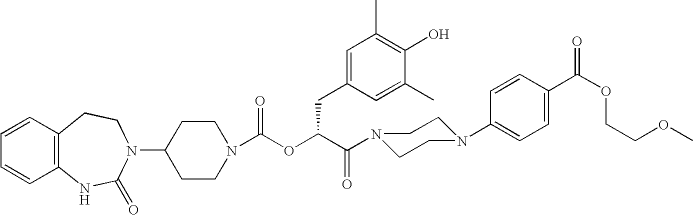 |
| (115) | 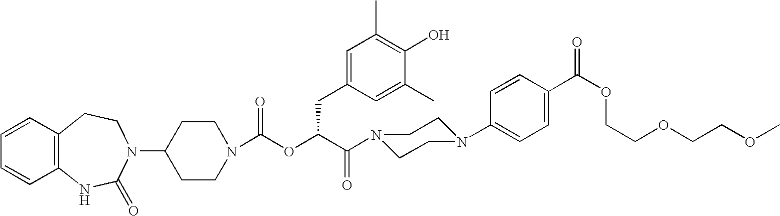 |
| (116) | 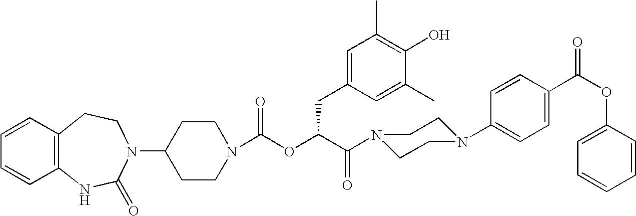 |
| (117) | 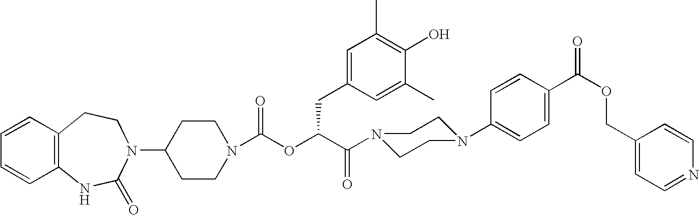 |
| (118) | 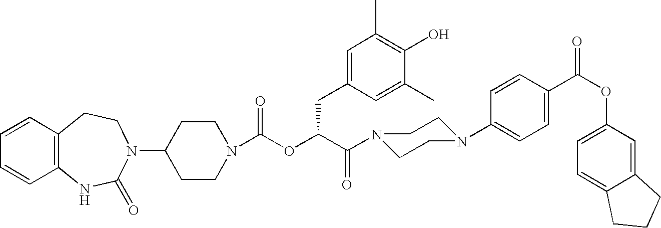 |
| (119) | 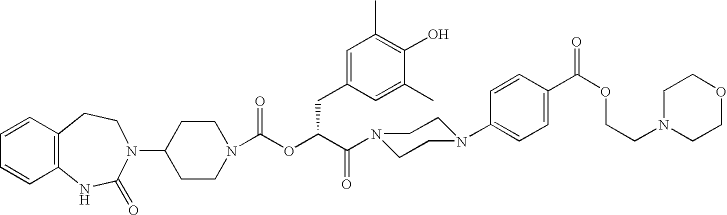 |
| (120) | 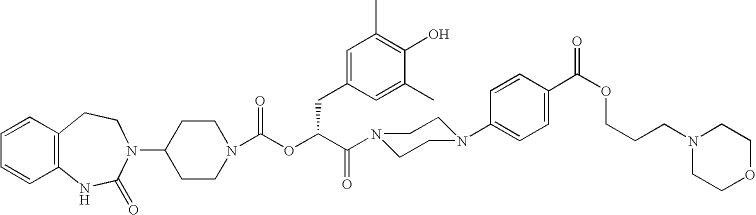 |
| (121) | 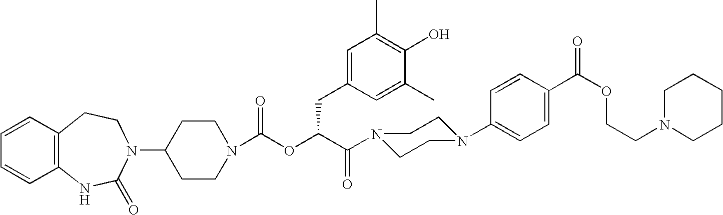 |
| (122) | 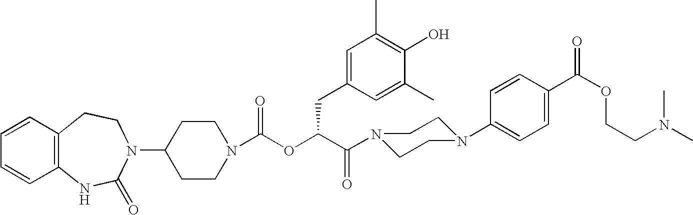 |
| (123) | 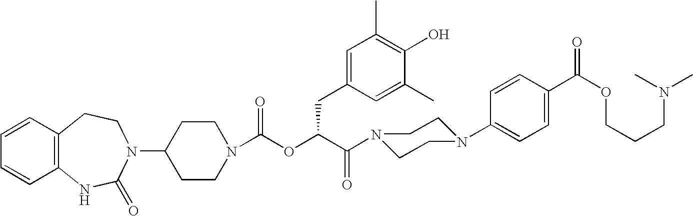 |
| (124) | 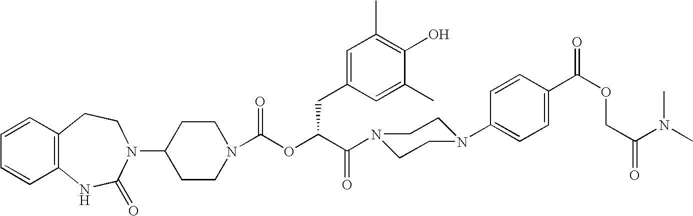 |
| (125) | 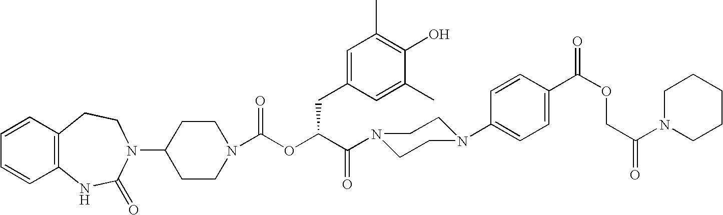 |
| (126) | 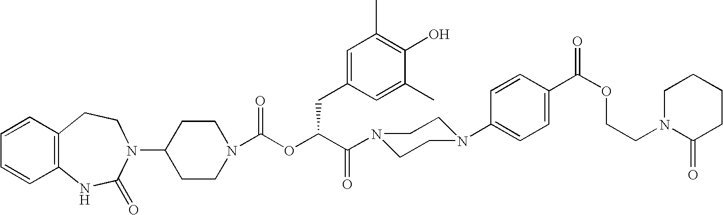 |
| (127) | 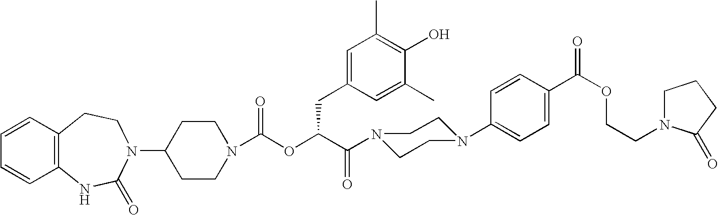 |
| (128) | 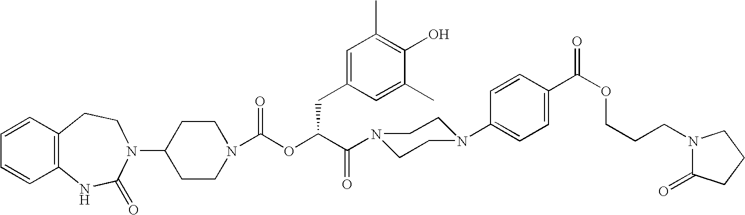 |
| (129) | 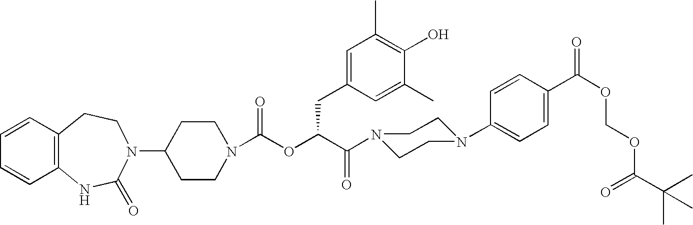 |
| (130) | 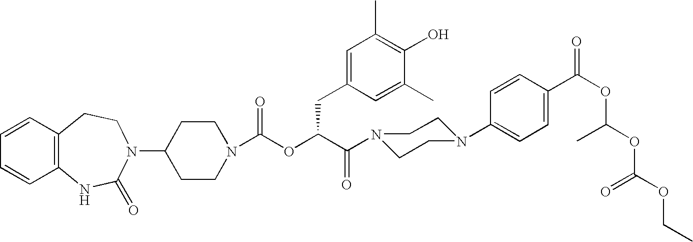 |
| (131) | 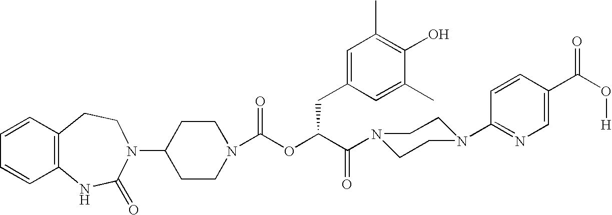 |
| (132) | 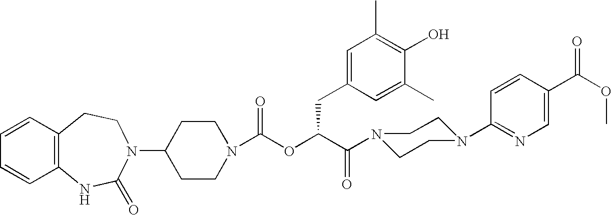 |
| (133) | 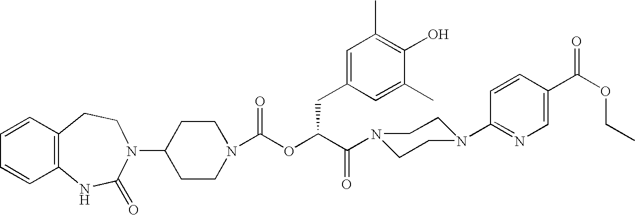 |
| (134) | 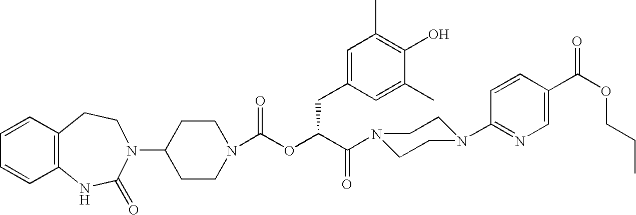 |
| (135) | 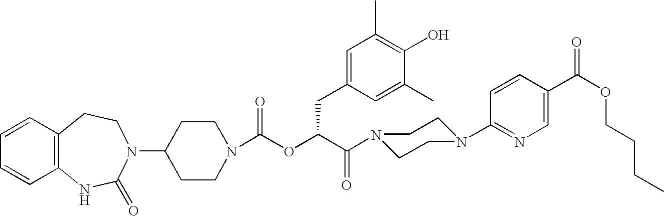 |
| (136) | 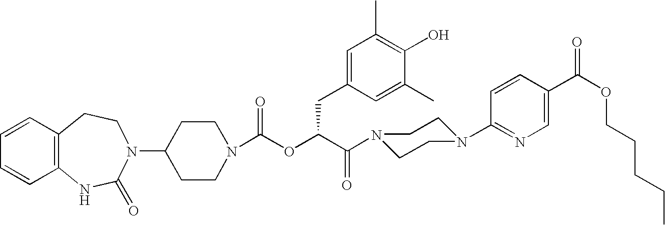 |
| (137) | 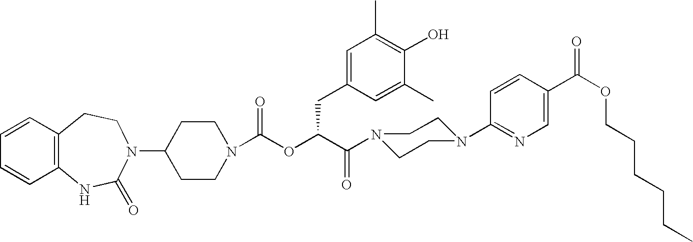 |
| (138) | 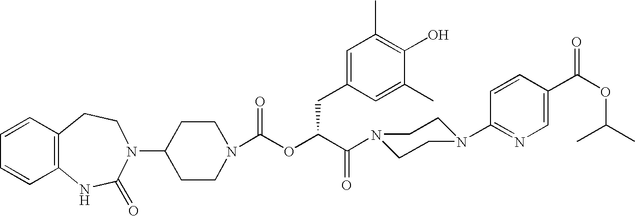 |
| (139) | 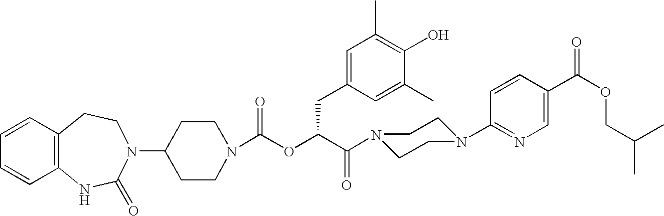 |
| (140) | 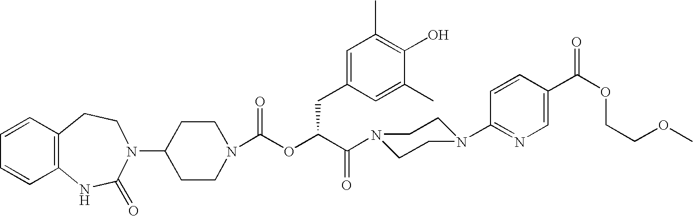 |
| (141) | 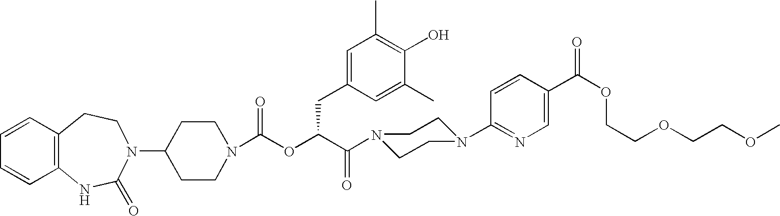 |
| (142) | 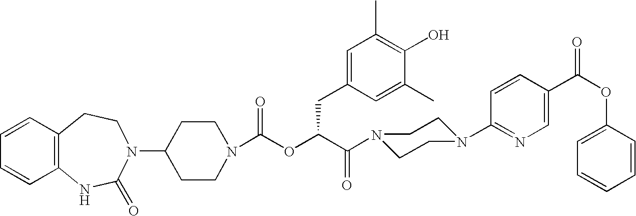 |
| (143) | 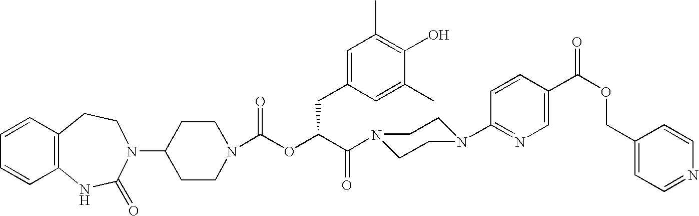 |
| (144) | 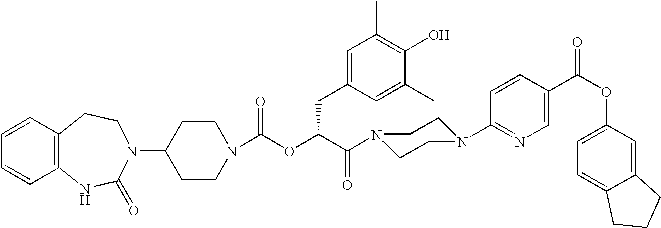 |
| (145) | 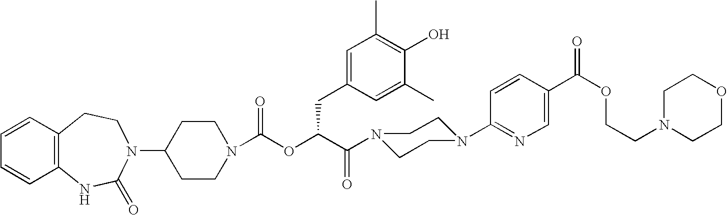 |
| (146) | 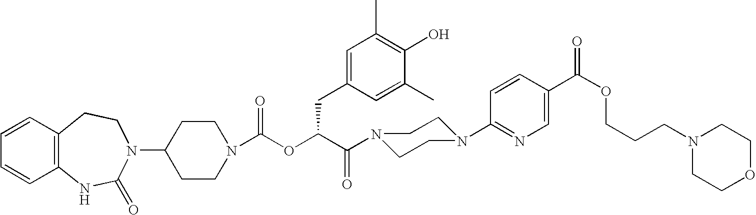 |
| (147) | 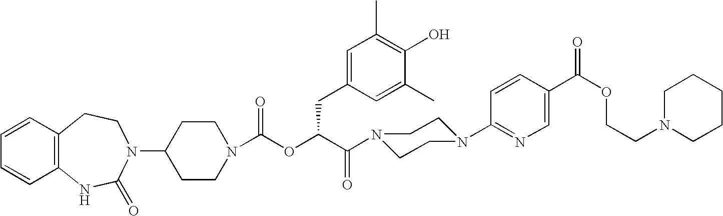 |
| (148) | 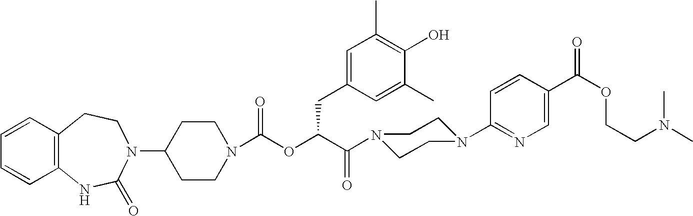 |
| (149) | 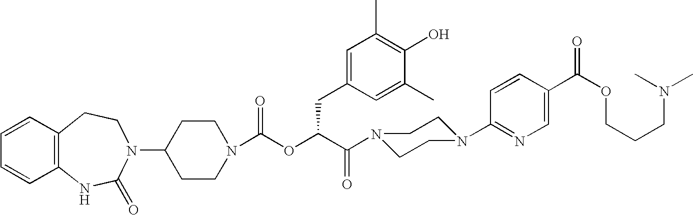 |
| (150) | 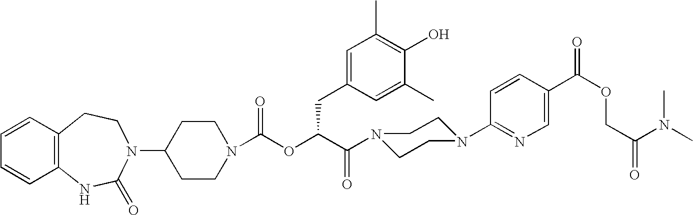 |
| (151) | 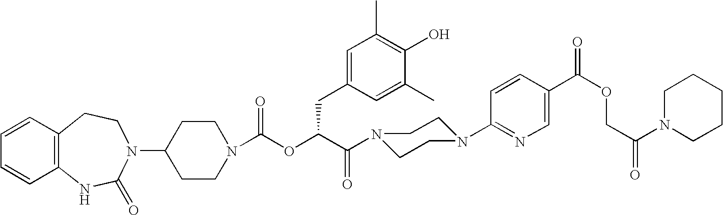 |
| (152) | 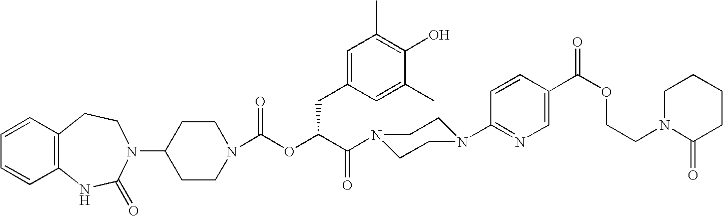 |
| (153) | 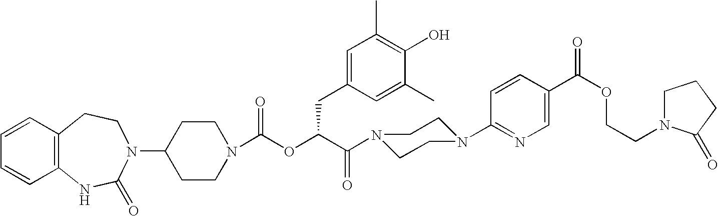 |
| (154) | 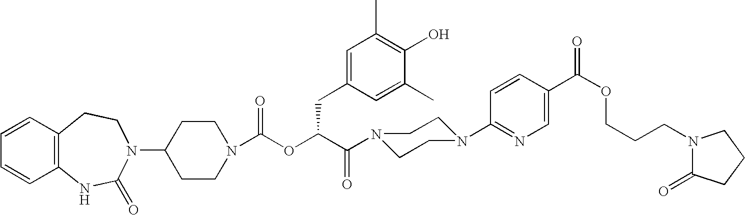 |
| (155) | 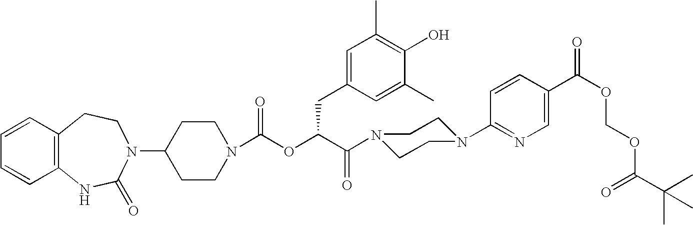 |
| (156) | 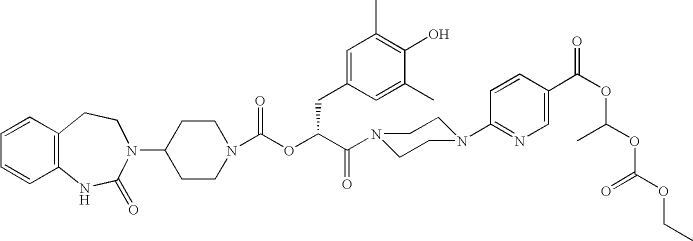 |
| (157) | 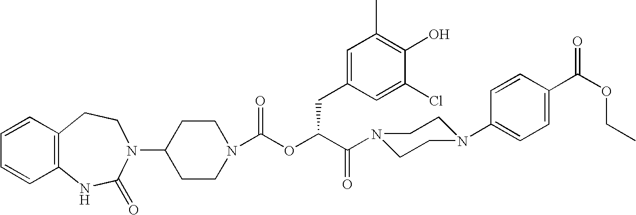 |
| (158) | 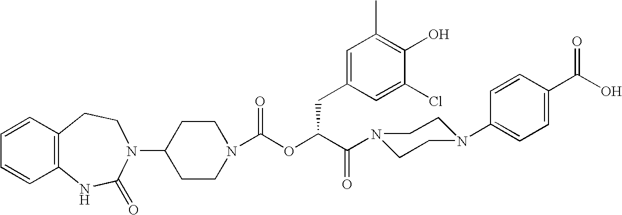 |
| (159) | 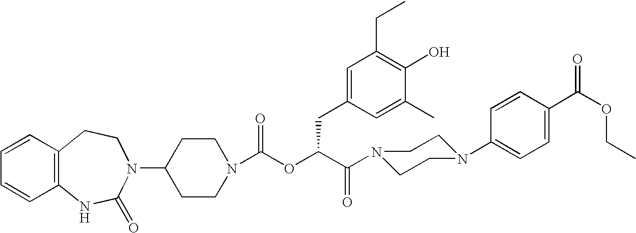 |
| (160) | 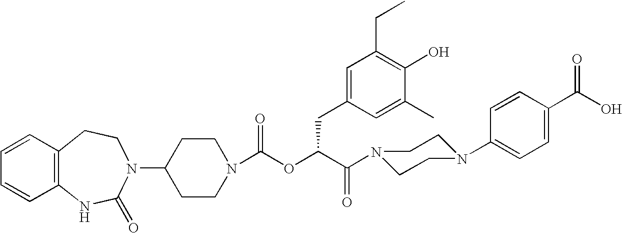 |
| (161) | 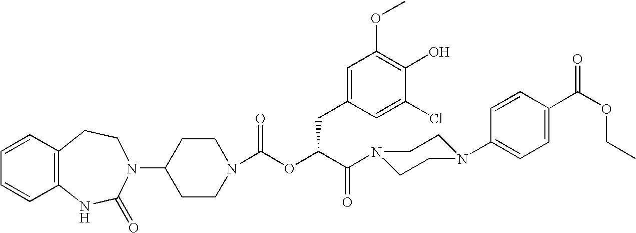 |
| (162) | 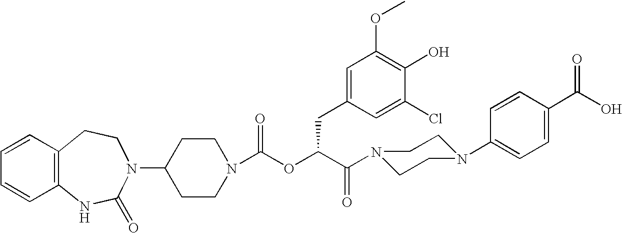 |
| (163) | 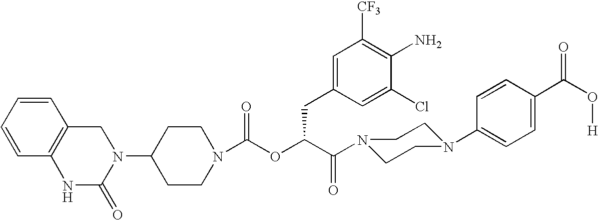 |
| (164) | 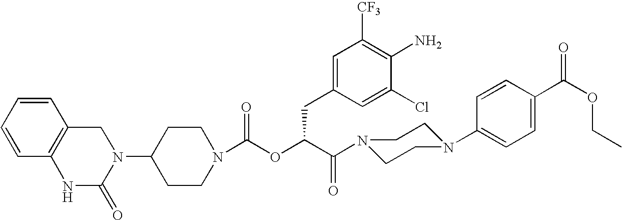 |
| (165) | 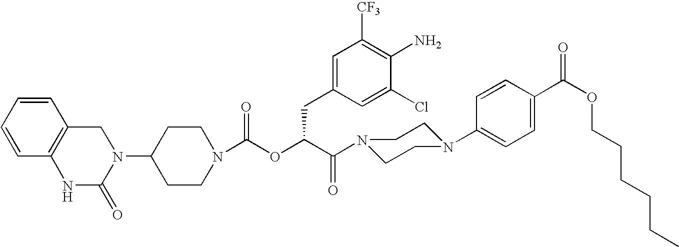 |
| (166) | 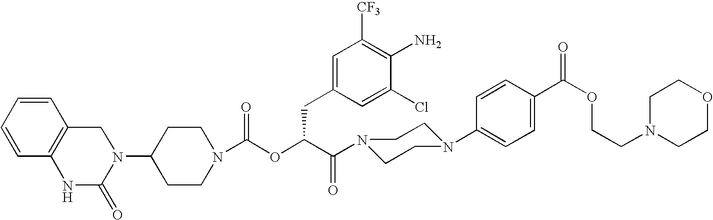 |
| (167) | 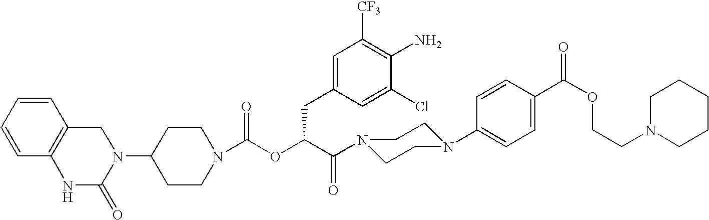 |
| (168) | 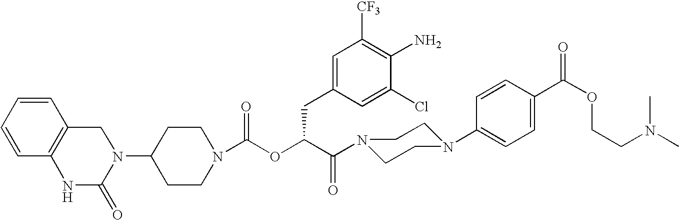 |
| (169) | 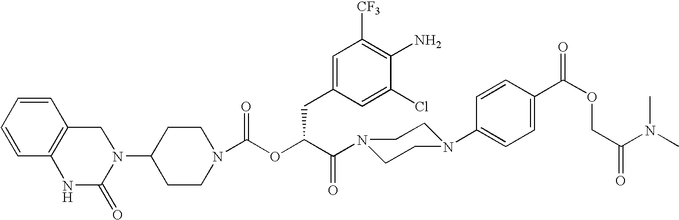 |
| (170) | 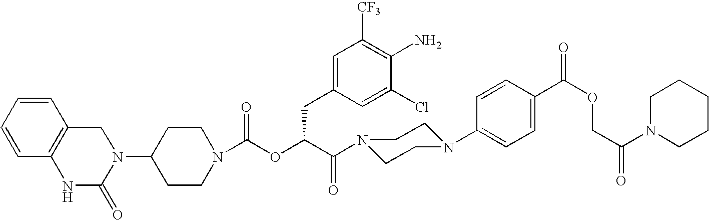 |
| (171) | 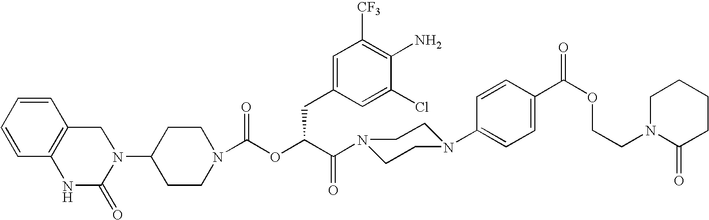 |
| (172) | 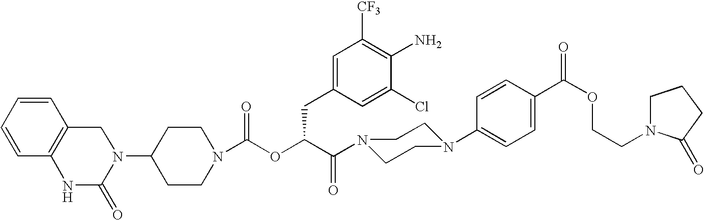 |
| (173) | 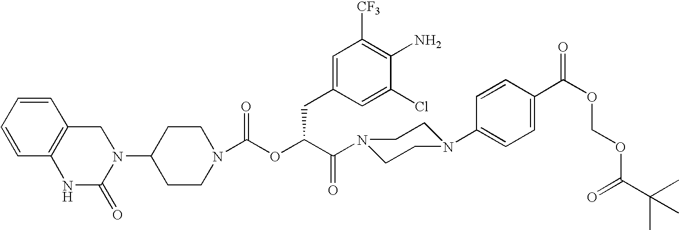 |
| (174) | 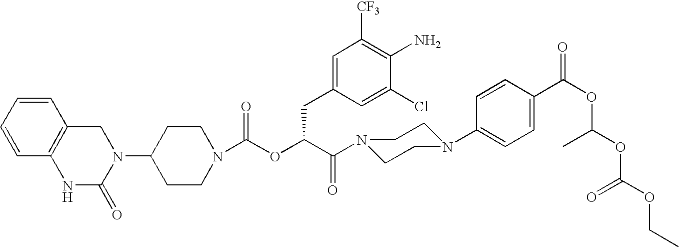 |
| (175) | 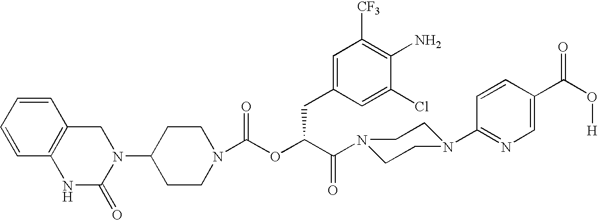 |
| (176) | 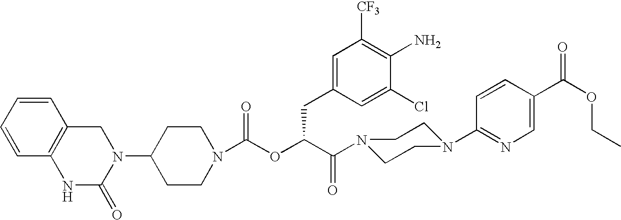 |
| (177) | 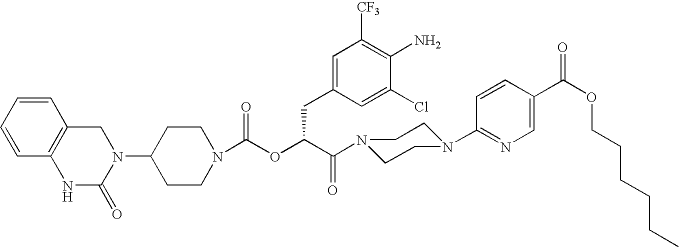 |
| (178) | 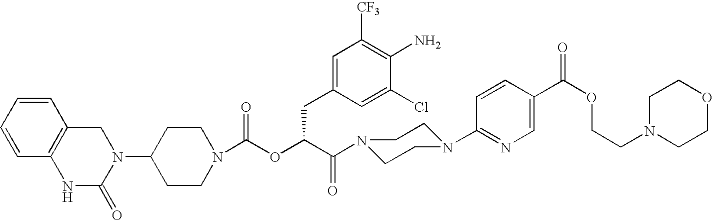 |
| (179) | 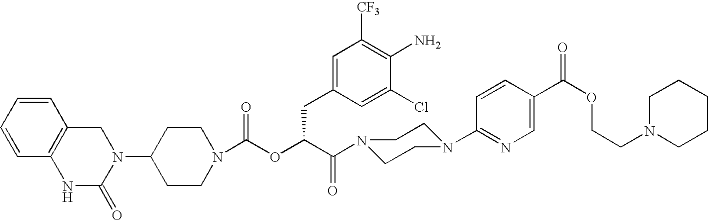 |
| (180) | 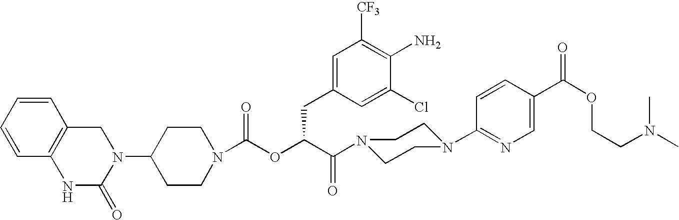 |
| (181) | 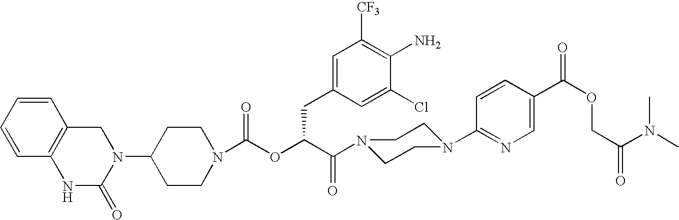 |
| (182) | 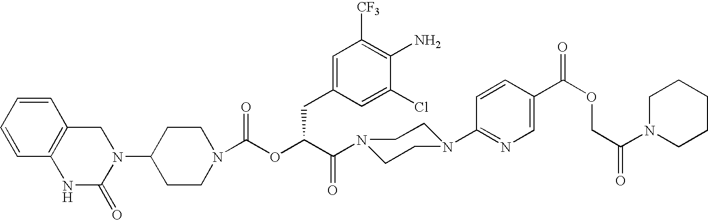 |
| (183) | 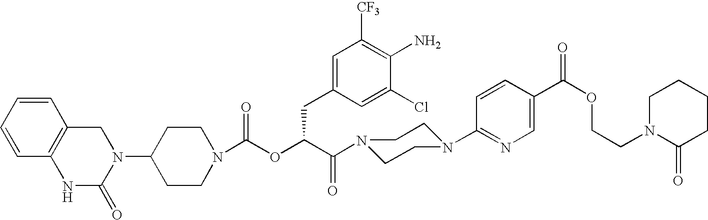 |
| (184) | 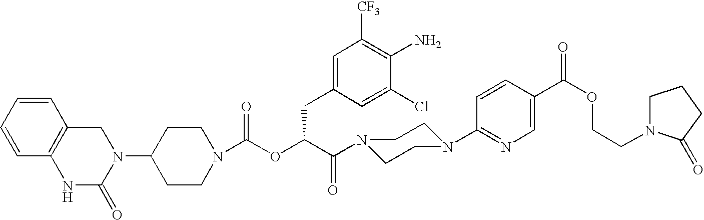 |
| (185) | 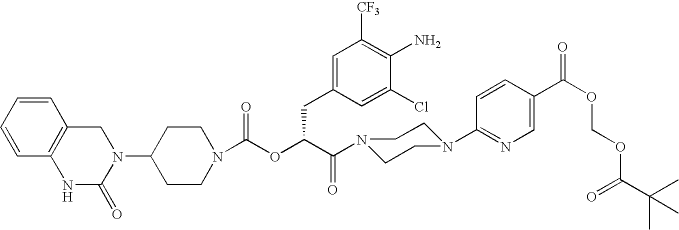 |
| (186) | 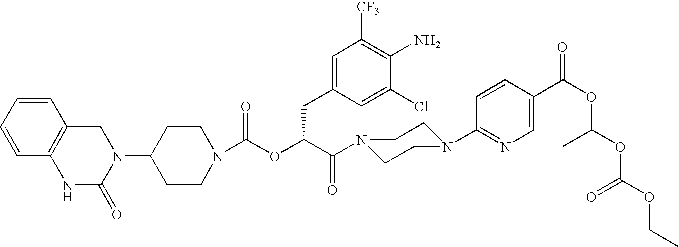 |
| (187) | 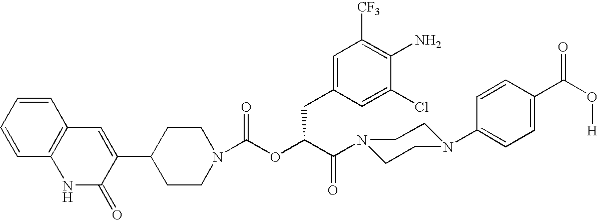 |
| (188) | 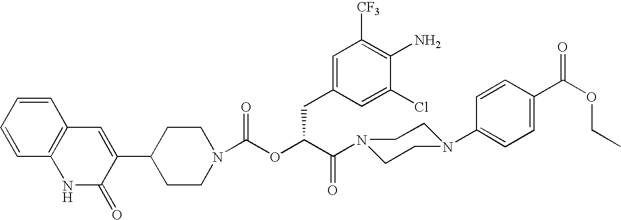 |
| (189) | 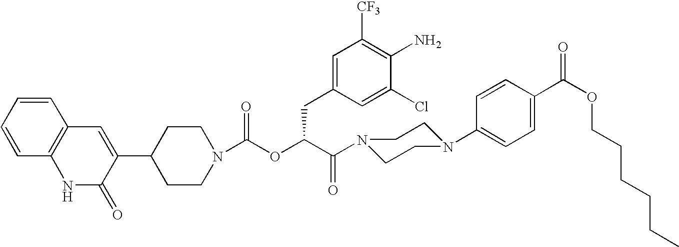 |
| (190) | 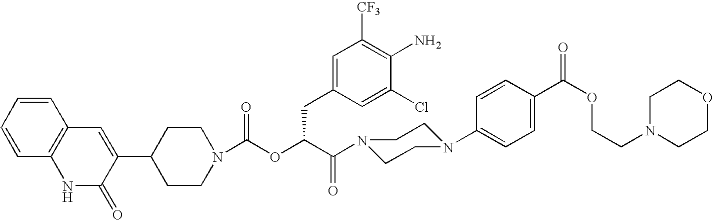 |
| (191) | 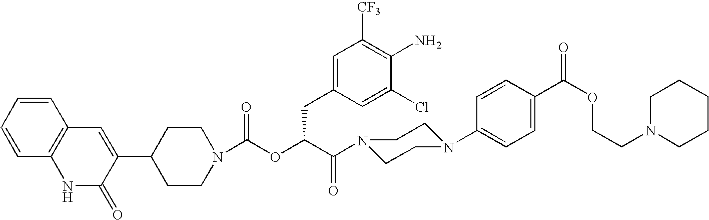 |
| (192) | 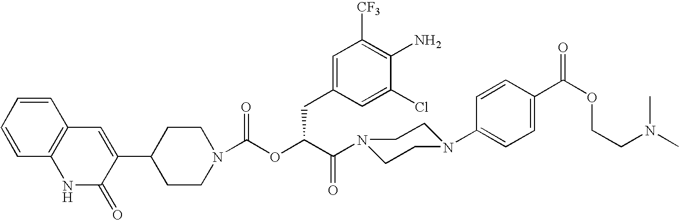 |
| (193) | 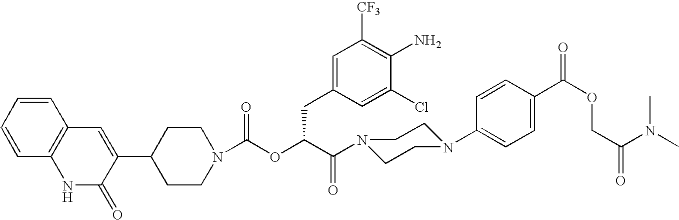 |
| (194) | 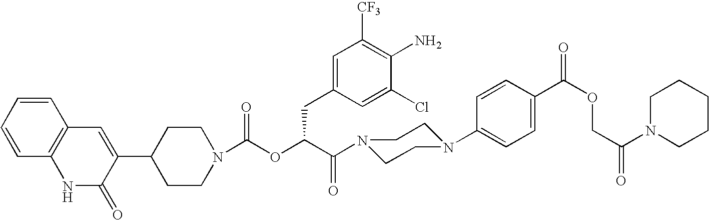 |
| (195) | 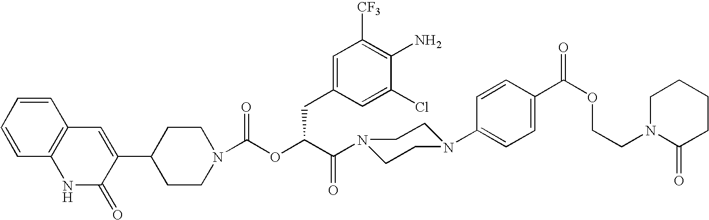 |
| (196) | 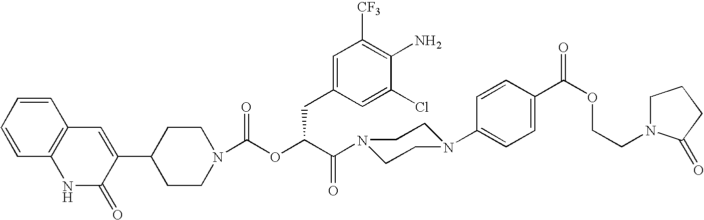 |
| (197) | 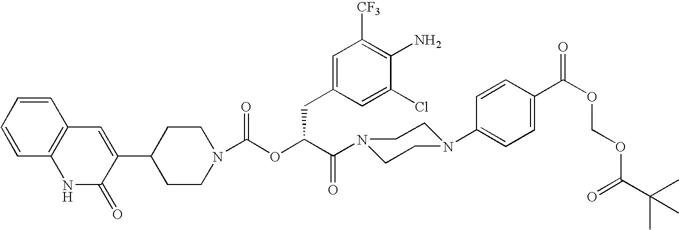 |
| (198) | 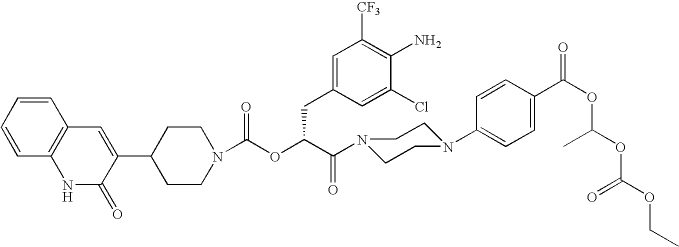 |
| (199) | 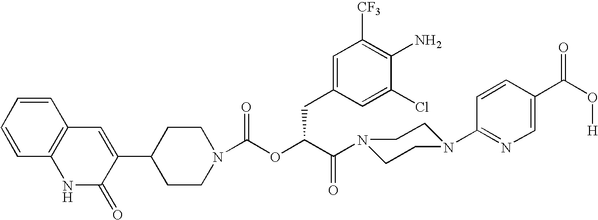 |
| (200) | 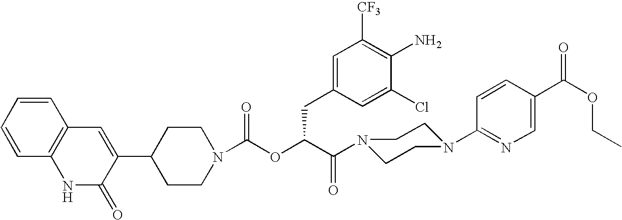 |
| (201) | 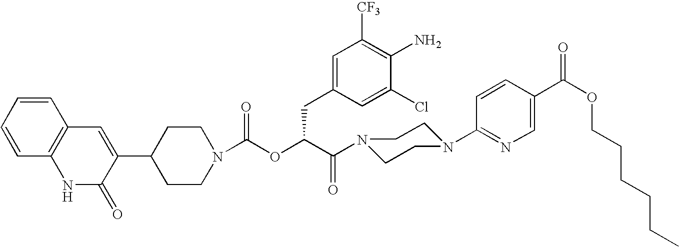 |
| (202) | 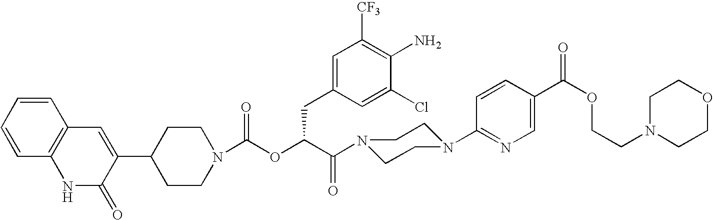 |
| (203) | 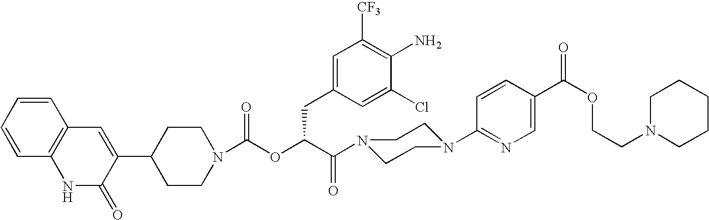 |
| (204) | 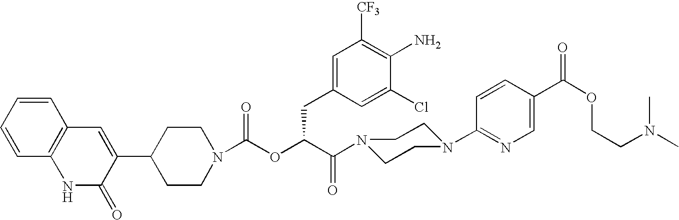 |
| (205) | 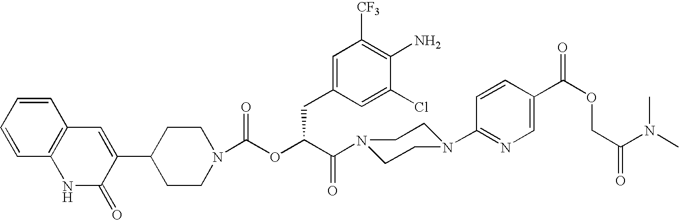 |
| (206) | 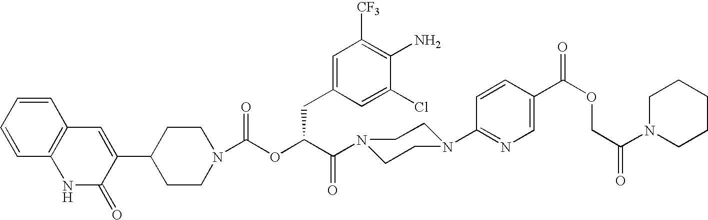 |
| (207) | 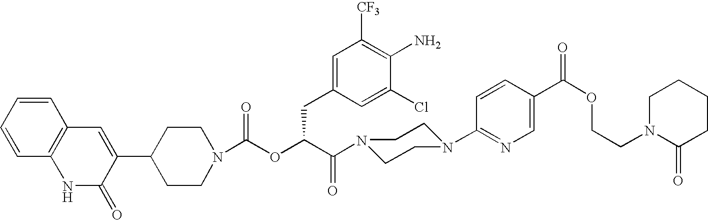 |
| (208) | 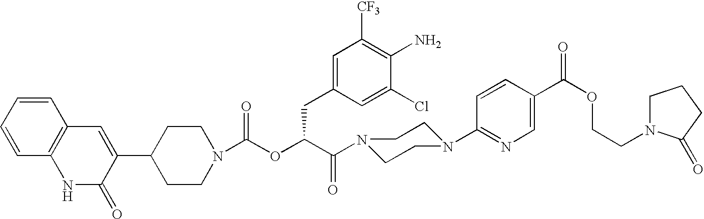 |
| (209) | 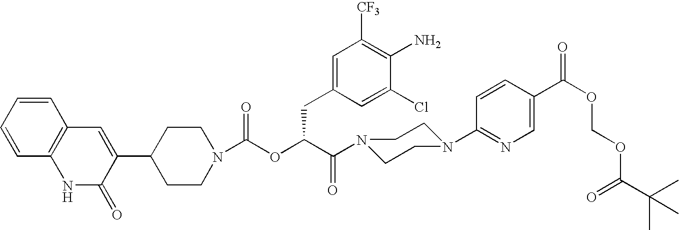 |
| (210) | 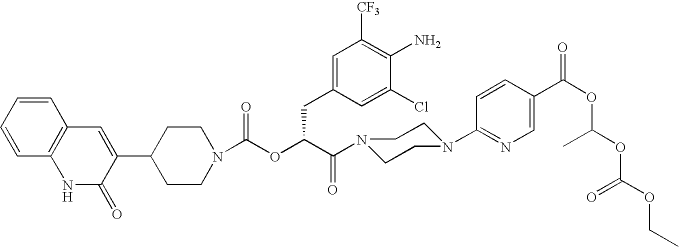 |
| (211) | 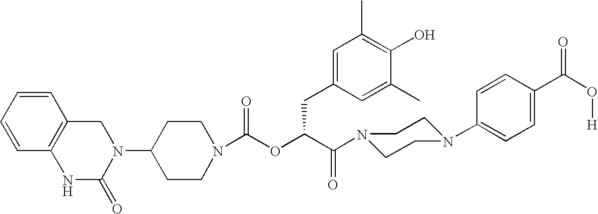 |
| (212) | 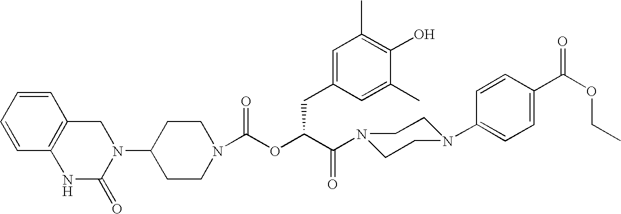 |
| (213) | 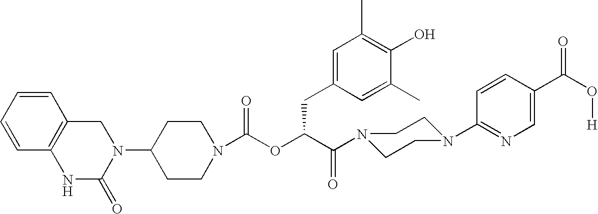 |
| (214) | 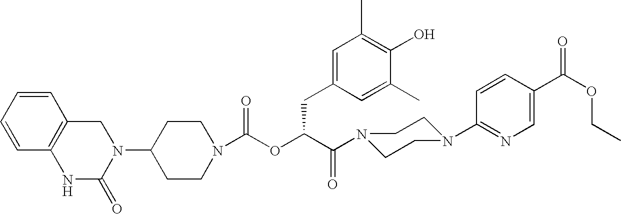 |
| (215) | 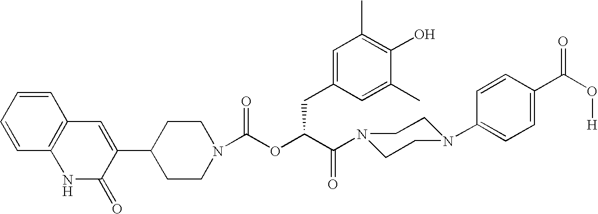 |
| (216) | 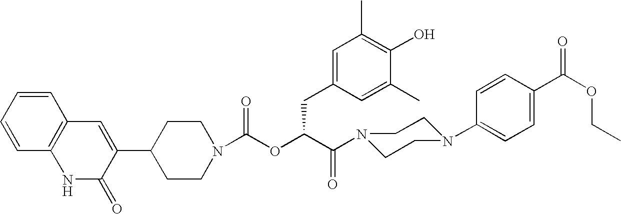 |
| (217) | 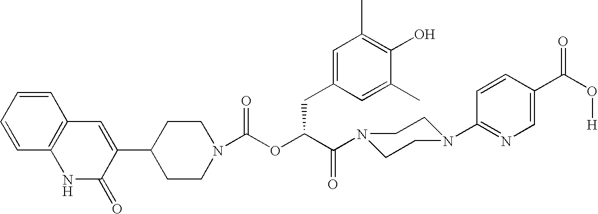 |
| (218) | 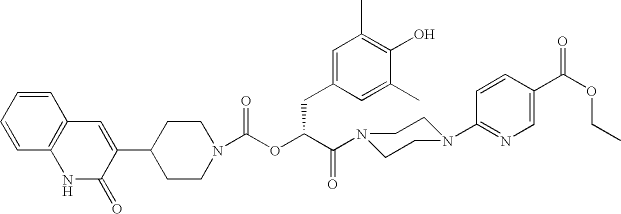 |

the tautomers, the diastereomers, the enantiomers, the hydrates, the mixtures thereof and the salts thereof and the hydrates of the salts, particularly the physiologically acceptable salts thereof with inorganic or organic acids or bases.

### TERMS AND DEFINITIONS USED

Unless otherwise stated, all the substituents are independent of one another. If for example there are a plurality of C₁₋₆-alkyl groups as substituents in one group, in the case of three C₁₋₆-alkyl substituents, independently of one another, one may represent methyl, one n-propyl and one tert-butyl.

Within the scope of this application, in the definition of possible substituents, these may also be represented in the form of a structural formula. If present, an asterisk (*) in the structural formula of the substituent is to be understood as being the linking point to the rest of the molecule.

The subject-matter of this invention also includes the compounds according to the invention, including the salts thereof, wherein one or more hydrogen atoms, for example one, two, three, four or five hydrogen atoms, are replaced by deuterium.

By the term “C₁₋₃-alkyl” (including those which are part of other groups) are meant branched and unbranched alkyl groups with 1 to 3 carbon atoms, by the term “C₁₋₆-alkyl” are meant branched and unbranched alkyl groups with 1 to 6 carbon atoms and by the term “C₁₋₈-alkyl” are meant branched and unbranched alkyl groups with 1 to 8 carbon atoms. Examples include: methyl, ethyl, n-propyl, iso-propyl, n-butyl, iso-butyl, sec-butyl, tert-butyl, n-pentyl, iso-pentyl, neo-pentyl, hexyl, heptyl and octyl. The following abbreviations may optionally also be used for the above-mentioned groups: Me, Et, n-Pr, i-Pr, n-Bu, i-Bu, t-Bu, etc. Unless stated otherwise, the definitions propyl, butyl, pentyl, hexyl, heptyl or octyl include all the possible isomeric forms of the groups in question. Thus, for example, propyl includes n-propyl and iso-propyl, butyl includes iso-butyl, sec-butyl and tert-butyl etc.

By the term “C₁₋₃-alkylene” (including those which are part of other groups) are meant branched and unbranched alkylene groups with 1 to 3 carbon atoms and by the term “C₂₋₄-alkylene” are meant branched and unbranched alkylene groups with 2 to 4 carbon atoms. Examples include: methylene, ethylene, propylene, 1-methylethylene, butylene, 1-methylpropylene, 1,1-dimethylethylene, 1,2-dimethylethylene. Unless stated otherwise, the definitions propylene and butylene include all the possible isomeric forms with the same number of carbons. Thus, for example, propyl also includes 1-methylethylene and butylene includes 1-methylpropylene, 1,1-dimethylethylene, 1,2-dimethylethylene.

It should also be mentioned that within the scope of the present invention the terms “alkylene” and “alkylenyl” are used synonymously.

“Halogen” within the scope of the present invention denotes fluorine, chlorine, bromine or iodine. Unless stated to the contrary, fluorine, chlorine and bromine are regarded as preferred halogens.

Compounds of general formula I may have acid groups, mainly carboxyl groups, and/or basic groups such as e.g. amino functions. Compounds of general formula I may therefore be present as internal salts, as salts with pharmaceutically useable inorganic acids such as for example hydrobromic acid, phosphoric acid, nitric acid, hydrochloric acid, sulphuric acid, methanesulphonic acid, ethanesulphonic acid, benzenesulphonic acid, p-toluenesulphonic acid or organic acids such as for example malic acid, succinic acid, acetic acid, fumaric acid, maleic acid, mandelic acid, lactic acid, tartaric acid, citric acid or as salts with pharmaceutically useable bases such as alkali or alkaline earth metal hydroxides, e.g. sodium hydroxide or potassium hydroxide, or carbonates, ammonia, zinc or ammonium hydroxides or organic amines such as e.g. diethylamine, triethylamine, ethanolamine, diethanolamine, triethanolamine, cyclohexylamine, dicyclohexylamine, inter alia.

The invention relates to the compounds in question, optionally in the form of the individual optical isomers, mixtures of the individual enantiomers or racemates, in the form of the tautomers as well as in the form of the free bases or the corresponding acid addition salts with pharmacologically acceptable acids—such as for example acid addition salts with hydrohalic acids—for example hydrochloric or hydrobromic acid—or organic acids—such as for example oxalic, fumaric, diglycolic or methanesulphonic acid.

The compounds according to the invention may be present as racemates, provided that they have only one chiral element, but may also be obtained as pure enantiomers, i.e. in the (R) or (S) form. Compounds which are present as racemates or in the (R) form are preferred.

However, the application also includes the individual diastereomeric pairs of antipodes or mixtures thereof, which are obtained if there is more than one chiral element in the compounds of general formula I, as well as the individual optically active enantiomers of which the above-mentioned racemates are made up.

The invention relates to the compounds in question, optionally in the form of the individual optical isomers, mixtures of the individual enantiomers or racemates, in the form of the tautomers as well as in the form of the free bases or the corresponding acid addition salts with pharmacologically acceptable acids—such as for example acid addition salts with hydrohalic acids—for example hydrochloric or hydrobromic acid—or organic acids—such as for example oxalic, fumaric, diglycolic or methanesulphonic acid.

### Methods of Preparation

The compounds of general formula I are prepared by methods known in principle. The following methods have proved particularly useful for preparing the compounds of general formula I according to the invention:

(a) For preparing compounds of general formula I wherein all the groups are as hereinbefore defined:
coupling a carboxylic acid of general formula IV

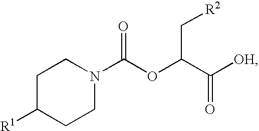

wherein R¹ and R² are as hereinbefore defined, with an amine of general formula V

H—R³—R⁴—R⁵

wherein R³, R⁴ and R⁵ are as hereinbefore defined, the linking taking place via the nitrogen atom of R³.

Before the reaction is carried out any carboxylic acid functions, primary or secondary amino functions or hydroxy functions present in the groups of the amine of formula H—R³—R⁴—R⁵ may be protected by conventional protective groups and after the reaction has taken place any protective groups used may be cleaved again using methods familiar to those skilled in the art.

The coupling is preferably carried out using methods known from peptide chemistry (cf. e.g. Houben-Weyl, Methoden der Organischen Chemie, Vol. 15/2), for example using carbodiimides such as e.g. dicyclohexylcarbodiimide (DCC), diisopropyl carbodiimide (DIC) or ethyl-(3-dimethylaminopropyl)-carbodiimide, O-(1H-benzotriazol-1-yl)-N,N—N′,N′-tetramethyluronium hexafluorophosphate (HBTU) or tetrafluoroborate (TBTU) or 1H-benzotriazol-1-yl-oxy-tris-(dimethylamino)-phosphonium hexafluorophosphate (BOP). By adding 1-hydroxybenzotriazole (HOBt) or 3-hydroxy-4-oxo-3,4-dihydro-1,2,3-benzotriazine (HOObt) the reaction speed can be increased. The couplings are normally carried out with equimolar amounts of the coupling components as well as the coupling reagent in solvents such as dichloromethane, tetrahydrofuran, acetonitrile, dimethyl formamide (DMF), dimethyl acetamide (DMA), N-methylpyrrolidone (NMP) or mixtures thereof and at temperatures between −30° C. and +30° C., preferably −20° C. and +25° C. If necessary, N-ethyl-diisopropylamine (Hünig base) is preferably used as an additional auxiliary base.

The so-called “anhydride process” is used as a further coupling method for synthesising compounds of general formula I (cf. also: M. Bodanszky, “Peptide Chemistry”, Springer-Verlag 1988, p. 58-59; M. Bodanszky, “Principles of Peptide Synthesis”, Springer-Verlag 1984, p. 21-27). The Vaughan variant of the “mixed anhydride process” is preferred (J. R. Vaughan Jr., J. Amer. Chem. Soc. 73, 3547 (1951)), in which the mixed anhydride of the carboxylic acid of general formula V which is to be coupled and monoisobutyl carbonate is obtained, using isobutyl chlorocarbonate in the presence of bases such as 4-methylmorpholine or 4-ethylmorpholine. The preparation of this mixed anhydride and the coupling with the amines of general formula VI are carried out in a one-pot process, using the above-mentioned solvents and at temperatures between −20° C. and +25° C., preferably 0° C. and +25° C.

(b) For preparing compounds of general formula I wherein all the groups are as hereinbefore defined:
coupling a compound of general formula VI

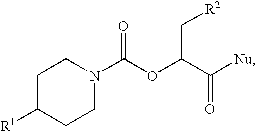

wherein R¹ and R² are as hereinbefore defined and Nu denotes a leaving group, for example a halogen atom, such as the chlorine, bromine or iodine atom, an alkylsulphonyloxy group with 1 to 10 carbon atoms in the alkyl moiety, a phenyl-sulphonyloxy or naphthylsulphonyloxy group optionally mono-, di- or trisubstituted by chlorine or bromine atoms, by methyl or nitro groups, wherein the substituents may be identical or different, a 1H-imidazol-1-yl, a 1H-pyrazol-1-yl optionally substituted by one or two methyl groups in the carbon skeleton, a 1H-1,2,4-triazol-1-yl, 1H-1,2,3-triazol-1-yl, 1H-1,2,3,4-tetrazol-1-yl, a vinyl, propargyl, p-nitrophenyl, 2,4-dinitrophenyl, trichlorophenyl, pentachlorophenyl, pentafluorophenyl, pyranyl or pyridinyl, a dimethylaminyloxy, 2(1H)-oxopyridin-1-yl-oxy, 2,5-dioxo-pyrrolidin-1-yloxy, phthalimidyloxy, 1H-benzotriazol-1-yloxy or azide group, with an amine of general formula V

H—R³—R⁴—R⁵

wherein all the groups are as hereinbefore defined and the link is effected via the nitrogen atom of the amine R³.

Before the reaction is carried out any carboxylic acid functions, primary or secondary amino functions or hydroxy functions present in the groups of the amine of general formula V may be protected by conventional protective groups and after the reaction has taken place any protective groups used may be cleaved again using methods familiar to those skilled in the art.

The reaction is carried out under Schotten-Baumann or Einhorn conditions, i.e. The components are reacted in the presence of at least one equivalent of an auxiliary base at temperatures between −50° C. and +120° C., preferably −10° C. and +30° C., and optionally in the presence of solvents. The auxiliary bases used are preferably alkali metal and alkaline earth metal hydroxides, e.g. sodium hydroxide, potassium hydroxide or barium hydroxide, alkali metal carbonates, e.g. sodium carbonate, potassium carbonate or caesium carbonate, alkali metal acetates, e.g. sodium or potassium acetate, as well as tertiary amines, e.g. pyridine, 2,4,6-trimethylpyridine, quinoline, triethylamine, N-ethyl-diisopropylamine, N-ethyl-dicyclohexylamine, 1,4-diazabicyclo[2,2,2]octane or 1,8-diazabicyclo[5,4,0]undec-7-ene, the solvents used may be, for example, dichloromethane, tetrahydrofuran, 1,4-dioxane, acetonitrile, dimethyl formamide, dimethyl acetamide, N-methyl-pyrrolidone or mixtures thereof; if alkali metal or alkaline earth metal hydroxides, alkali metal carbonates or acetates are used as the auxiliary bases, water may also be added to the reaction mixture as cosolvent.

The new compounds of general formula I according to the invention contain one or more chiral centres. If for example there are two chiral centres present, the compounds may occur in the form of two diastereomeric pairs of antipodes. The invention includes the individual isomers as well as the mixtures thereof.

The diastereomers may be separated on the basis of their different physico-chemical properties, e.g. by fractional crystallisation from suitable solvents, by high pressure liquid or column chromatography, using chiral or preferably non-chiral stationary phases.

Racemates covered by general formula I may be separated for example by HPLC on suitable chiral stationary phases (e.g. chiral AGP, Chiralpak AD). Racemates which contain a basic or acidic function can also be separated via the diastereomeric, optically active salts which are produced on reacting with an optically active acid, for example (+) or (−)-tartaric acid, (+) or (−)-diacetyl tartaric acid, (+) or (−)-monomethyl tartrate or (+) or (−)-camphorsulphonic acid, or an optically active base, for example with (R)-(+)-1-phenylethylamine, (S)-(−)-1-phenylethylamine or (S)-brucine.

According to a conventional method of separating isomers, the racemate of a compound of general formula I is reacted with one of the abovementioned optically active acids or bases in equimolar amounts in a solvent and the resulting crystalline, diastereomeric, optically active salts thereof are separated using their different solubilities. This reaction may be carried out in any type of solvent provided that it is sufficiently different in terms of the solubility of the salts. Preferably, methanol, ethanol or mixtures thereof, for example in a ratio by volume of 50:50, are used. Then each of the optically active salts is dissolved in water, carefully neutralised with a base such as sodium carbonate or potassium carbonate, or with a suitable acid, e.g. with dilute hydrochloric acid or aqueous methanesulphonic acid, and in this way the corresponding free compound is obtained in the (+) or (−) form.

The (R) or (S) enantiomer alone or a mixture of two optically active diastereomeric compounds covered by general formula I may also be obtained by performing the syntheses described above with a suitable reaction component in the (R) or (S) configuration.

The hydroxycarboxylic acids of general formula V needed as starting compounds may be obtained by reacting piperidines of general formula VII

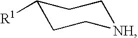

wherein R¹ is as hereinbefore defined, with carbonic acid derivatives of general formula VIII

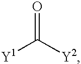

wherein Y¹ and Y² represent nucleofugic groups, which may be identical or different, preferably the chlorine atom, the p-nitrophenoxy or trichloromethoxy group,
and with compounds of general formula IX

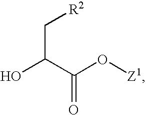

wherein R² is as hereinbefore defined and Z¹ denotes a protective group for a carboxy group, for example a C₁₋₆-alkyl or an optionally substituted benzyl group, wherein the alkyl groups may be straight-chain or branched and the benzyl group may be substituted by one or two methoxy groups.

Preferably Z¹ denotes the methyl, ethyl, tert-butyl or benzyl group. Before the reaction is carried out any hydroxy functions present in the group R² of a compound of formula (VI) may be protected by conventional protective groups and after the reaction is complete any protective groups used may be cleaved again using methods familiar to the skilled man.

In a first step the compounds of general formula VII are reacted with the carbonic acid derivatives of general formula VIII in a solvent, for example in dichloromethane, THF, pyridine or mixtures thereof, at a temperature between −20° C. to 50° C. in the presence of a base, for example triethylamine, pyridine or ethyldiisopropylamine. The intermediate thus formed may be purified or reacted further without purification. The reaction of these intermediates with compounds of general formula IX also takes place in one of the above-mentioned solvents and at the temperatures specified above, in the presence of a base, such as triethylamine or pyridine, with or without the addition of an activating reagent, such as e.g. 4-dimethylaminopyridine. To activate them the compounds of general formula IX may also be deprotonated using a metal hydride, such as e.g. NaH or KH, while in this case there is no need for the base or the activating reagent to be present.

The starting compounds of formula VII and VIII are either commercially obtainable, known from the literature or may be prepared using methods known from the literature.

One way of obtaining compounds of general formula IX comprises reacting aldehydes of general formula X

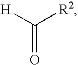

wherein R² is as hereinbefore defined, with N-acetylglycine in acetic anhydride as solvent in the presence of alkali metal acetate, preferably sodium or potassium acetate, at suitable temperatures, preferably at 80 to 130° C.

The azlactones obtained as primary product are hydrolysed without being isolated to form the compounds of general formula XI

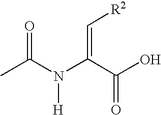

wherein R² is as hereinbefore defined. By further reaction in the presence of aqueous inorganic acids, such as sulphuric, phosphoric or hydrochloric acid, but preferably hydrochloric acid, compounds of general formula XII are obtained

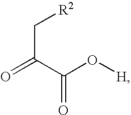

wherein R² is as hereinbefore defined.

These are then converted with suitable reducing agents into the compounds of general formula XIII

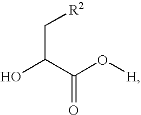

wherein R² is as hereinbefore defined.

Suitable reducing agents are alkali metal borohydrides, such as sodium or potassium borohydride. Other suitable reducing agents are chlorodialkylboranes, such as chlorodicyclohexylborane. If chiral chlorodialkylboranes, such as e.g. B-chlorodiisopinocampheylborane, are used, the compounds of general formula XIII may be isolated in enantiomerically pure form. The further reaction of compounds of general formula XIII to form compounds of general formula IX is carried out in an alcoholic medium, preferably in methanol or ethanol, in the presence of a suitable acid, such as hydrochloric acid. Alternatively, the reaction may be carried out by reacting with thionyl chloride in alcoholic solvents, preferably methanol.

All the compounds of general formula I which contain primary or secondary amino, hydroxy or hydroxycarbonyl functions are preferably obtained from precursors with protective groups. Examples of protective groups for amino functions include a benzyloxycarbonyl, 2-nitrobenzyloxycarbonyl, 4-nitro-benzyloxycarbonyl, 4-methoxy-benzyloxycarbonyl, 2-chloro-benzyloxycarbonyl, 3-chloro-benzyloxycarbonyl, 4-chloro-benzyloxycarbonyl, 4-biphenylyl-α,α-dimethyl-benzyl-oxycarbonyl or 3,5-dimethoxy-α,α-dimethyl-benzyloxycarbonyl group, an alkoxycarbonyl group with a total of 1 to 5 carbon atoms in the alkyl moiety, for example the methoxycarbonyl, ethoxycarbonyl, n-propoxycarbonyl, iso-propoxycarbonyl, n-butoxycarbonyl, 1-methyl propoxycarbonyl, 2-methyl propoxy-carbonyl or tert-butyloxycarbonyl group, the allyloxycarbonyl, 2,2,2-trichloro-(1,1-dimethylethoxy)carbonyl or 9-fluorenylmethoxycarbonyl group or a formyl, acetyl or trifluoroacetyl group.

Examples of protective groups for hydroxy functions include a trimethylsilyl, triethylsilyl, triisopropyl, tert-butyldimethylsilyl or tert-butyldiphenylsilyl group, a tert-butyl, benzyl, 4-methoxybenzyl or 3,4-dimethoxybenzyl group.

Examples of protective groups for hydroxycarbonyl functions include an alkyl group with a total of 1 to 5 carbon atoms, for example the methyl, ethyl, n-propyl, isopropyl, n-butyl, tert-butyl, allyl, 2,2,2-trichloroethyl, benzyl or 4-methoxybenzyl group.

The compounds of general formula I obtained may, if they contain suitable basic functions, be converted, particularly for pharmaceutical use, into their physiologically acceptable salts with inorganic or organic acids. Suitable acids include for example hydrochloric acid, hydrobromic acid, phosphoric acid, nitric acid, sulphuric acid, methanesulphonic acid, ethanesulphonic acid, benzenesulphonic acid, p-toluenesulphonic acid, acetic acid, fumaric acid, succinic acid, lactic acid, mandelic acid, malic acid, citric acid, tartaric acid or maleic acid.

Moreover, if they contain a carboxylic acid function, the new compounds of formula I may be converted into the addition salts thereof with inorganic or organic bases, particularly, for pharmaceutical use, into their physiologically acceptable addition salts. Suitable bases for this include for example sodium hydroxide, potassium hydroxide, ammonia, cyclohexylamine, dicyclohexylamine, ethanolamine, diethanolamine and triethanolamine.

The present invention relates to racemates if the compounds of general formula I have only one chiral element. However, the application also includes the individual diastereomeric pairs of antipodes or mixtures thereof which are obtained if there is more than one chiral element in the compounds of general formula I, as well as the individual optically active enantiomers of which the above-mentioned racemates are made up.

Also included in the subject matter of this invention are the compounds according to the invention, including the salts thereof, in which one or more hydrogen atoms, for example one, two, three, four or five hydrogen atoms, are replaced by deuterium.

The new compounds of general formula I and the physiologically acceptable salts thereof have valuable pharmacological properties, based on their selective CGRP-antagonistic properties. The invention further relates to pharmaceutical compositions containing these compounds, their use and the preparation thereof.

The new compounds mentioned above and the physiologically acceptable salts thereof have CGRP-antagonistic properties and exhibit good affinities in CGRP receptor binding studies. The compounds display CGRP-antagonistic properties in the pharmacological test systems described hereinafter.

The following experiments were carried out to demonstrate the affinity of the above-mentioned compounds for human CGRP-receptors and their antagonistic properties:

A. Binding Studies with SK-N-MC cells (Expressing the Human CGRP Receptor)

SK-N-MC cells are cultivated in “Dulbecco's modified Eagle medium”. The medium is removed from confluent cultures. The cells are washed twice with PBS buffer (Gibco 041-04190 M), detached by the addition of PBS buffer mixed with 0.02% EDTA, and isolated by centrifuging. After resuspension in 20 ml of “Balanced Salts Solution” [BSS (in mM): NaCl 120, KCl 5.4, NaHCO₃ 16.2, MgSO₄ 0.8, NaHPO₄ 1.0, CaCl₂ 1.8, D-glucose 5.5, HEPES 30, pH 7.40] the cells are centrifuged twice at 100×g and resuspended in BSS. After the number of cells has been determined, the cells are homogenised using an Ultra-Turrax and centrifuged for 10 minutes at 3000×g. The supernatant is discarded and the pellet is recentrifuged in Tris buffer (10 mM Tris, 50 mM NaCl, 5 mM MgCl₂, 1 mM EDTA, pH 7.40) enriched with 1% bovine serum albumin and 0.1% bacitracin, and resuspended (1 ml/1000000 cells). The homogenised product is frozen at −80° C. The membrane preparations are stable for more than 6 weeks under these conditions.

After thawing, the homogenised product is diluted 1:10 with assay buffer (50 mM Tris, 150 mM NaCl, 5 mM MgCl₂, 1 mM EDTA, pH 7.40) and homogenised for 30 seconds with an Ultra-Turrax. 230 μl of the homogenised product are incubated for 180 minutes at ambient temperature with 50 pM ¹²⁵I-iodotyrosyl-Calcitonin-Gene-Related Peptide (Amersham) and increasing concentrations of the test substances in a total volume of 250 μl. The incubation is ended by rapid filtration through GF/B-glass fibre filters treated with polyethyleneimine (0.1%) using a cell harvester. The protein-bound radioactivity is measured using a gamma counter.

Non-specific binding is defined as the bound radioactivity after the presence of 1 μM human CGRP-alpha during incubation.

The concentration binding curves are analysed using computer-aided non-linear curve fitting.

The compounds mentioned hereinbefore show IC₅₀ values ≦10000 nM in the test described.

### B. CGRP Antagonism in SK-N-MC Cells

SK-N-MC cells (1 million cells) are washed twice with 250 μl incubation buffer (Hanks' HEPES, 1 mM 3-isobutyl-1-methylxanthine, 1% BSA, pH 7.4) and pre-incubated at 37° C. for 15 minutes. After the addition of CGRP (10 μl) as agonist in increasing concentrations (10⁻¹¹ to 10⁻⁶ M), or additionally the substance in 3 to 4 different concentrations, the mixture is incubated for another 15 minutes.

Intracellular cAMP is then extracted by the addition of 20 μl of 1M HCl and centrifugation (2000×g, 4° C., for 15 minutes). The supernatants are frozen in liquid nitrogen and stored at −20° C.

The cAMP contents of the samples are determined by radioimmunoassay (Messrs. Amersham) and the pA₂ values of antagonistically acting substances are determined graphically.

The compounds according to the invention exhibit CGRP-antagonistic properties in the in vitro test model described, in a dosage range between 10⁻¹² and 10⁻⁵ M.

### Indications

In view of their pharmacological properties the compounds according to the invention and the salts thereof with physiologically acceptable acids are thus suitable for the acute and prophylactic treatment of headaches, particularly migraine or cluster headaches and tension headaches. Moreover, the compounds according to the invention also have a positive effect on the following diseases: non-insulin-dependent diabetes mellitus (“NIDDM”), cardiovascular diseases, morphine tolerance, diarrhoea caused by clostridium toxin, skin diseases, particularly thermal and radiation-induced skin damage including sunburn, lichen, pruritis, pruritic toxidermies and severe itching, inflammatory diseases, e.g. inflammatory diseases of the joints (osteoarthritis, rheumatoid arthritis, neurogenic arthritis), generalised soft-tissue rheumatism (fibromyalgia), neurogenic inflammation of the oral mucosa, inflammatory lung diseases, allergic rhinitis, asthma, COPD, diseases accompanied by excessive vasodilatation and resultant reduced blood supply to the tissues, e.g. shock and sepsis, chronic pain, e.g. diabetic neuropathies, neuropathies induced by chemotherapy, HIV-induced neuropathies, postherpetic neuropathies, neuropathies induced by tissue trauma, trigeminal neuralgias, temporomandibular dysfunctions, CRPS (complex regional pain syndrome), back pain, and visceral complaints, such as e.g. Irritable bowel syndrome (IBS) and inflammatory bowel syndrome. In addition, the compounds according to the invention have a general pain-relieving effect. The symptoms of menopausal hot flushes caused by vasodilatation and increased blood flow in oestrogen-deficient women and hormone-treated patients with prostate carcinoma and castrated men are favourably affected by the CGRP antagonists of the present application in a preventive and acute-therapeutic capacity, this therapeutic approach being distinguished from hormone replacement by the absence of side effects.

Preferably, the compounds according to the invention are suitable for the acute and prophylactic treatment of migraine and cluster headaches, for treating irritable bowel syndrome (IBS) and for the preventive and acute-therapeutic treatment of hot flushes in oestrogen-deficient women.

The dosage required to achieve a corresponding effect is conveniently 0.0001 to 3 mg/kg of body weight, preferably 0.01 to 1 mg/kg of body weight, when administered intravenously or subcutaneously, and 0.01 to 10 mg/kg of body weight, preferably 0.1 to 10 mg/kg of body weight when administered orally, nasally or by inhalation, 1 to 3× a day in each case.

If the treatment with CGRP antagonists and/or CGRP release inhibitors is given as a supplement to conventional hormone replacement, it is advisable to reduce the doses specified above, in which case the dosage may be from 1/5 of the lower limits mentioned above up to 1/1 of the upper limits specified.

The invention further relates to the use of the compounds according to the invention as valuable adjuvants for the production and purification (by affinity chromatography) of antibodies as well as in RIA and ELISA assays, after suitable radioactive labelling, for example by tritiation of suitable precursors, for example by catalytic hydrogenation with tritium or replacing halogen atoms with tritium, and as a diagnostic or analytical adjuvant in neurotransmitter research.

### Combinations

Categories of active substance which may be used in combination include e.g. antiemetics, prokinetics, neuroleptics, antidepressants, neurokinin antagonists, anticonvulsants, histamine-H1-receptor antagonists, β-blockers, α-agonists and α-antagonists, ergot alkaloids, mild analgesics, non-steroidal antiphlogistics, corticosteroids, calcium antagonists, 5-HT_{1B/1D}-agonists or other anti-migraine agents which may be formulated together with one or more inert conventional carriers and/or diluents, e.g. with corn starch, lactose, glucose, microcrystalline cellulose, magnesium stearate, polyvinyl pyrrolidone, citric acid, tartaric acid, water, water/ethanol, water/glycerol, water/sorbitol, water/polyethylene glycol, propylene glycol, cetylstearyl alcohol, carboxymethylcellulose or fatty substances such as hard fat or suitable mixtures thereof, into conventional galenic preparations such as plain or coated tablets, capsules, powders, suspensions, solutions, metered dose aerosols or suppositories.

Thus other active substances which may be used for the combinations mentioned above include for example the non-steroidal antiinflammatories aceclofenac, acemetacin, acetylsalicylic acid, acetaminophen (paracetamol), azathioprine, diclofenac, diflunisal, fenbufen, fenoprofen, flurbiprofen, ibuprofen, indometacin, ketoprofen, leflunomide, lornoxicam, mefenamic acid, naproxen, phenylbutazone, piroxicam, sulphasalazine, zomepirac or the pharmaceutically acceptable salts thereof as well as meloxicam and other selective COX2-inhibitors, such as for example rofecoxib, valdecoxib, parecoxib, etoricoxib and celecoxib, as well as substances that inhibit earlier or later stages of prostaglandin synthesis or prostaglandin receptor antagonists such as e.g. EP2-receptor antagonists and IP-receptor antagonists.

It is also possible to use ergotamine, dihydroergotamine, metoclopramide, domperidone, diphenhydramine, cyclizine, promethazine, chlorpromazine, vigabatrin, timolol, isometheptene, pizotifen, botox, gabapentin, pregabalin, duloxetine, topiramate, riboflavin, montelukast, lisinopril, micardis, prochloroperazine, dexamethasone, flunarizine, dextropropoxyphene, meperidine, metoprolol, propranolol, nadolol, atenolol, clonidine, indoramin, carbamazepine, phenyloin, valproate, amitryptiline, imipramine, venlafaxine, lidocaine or diltiazem and other 5-HT_{1B/1D}-agonists such as, for example, almotriptan, avitriptan, eletriptan, frovatriptan, naratriptan, rizatriptan, sumatriptan and zolmitriptan.

Furthermore, CGRP antagonists with vanilloid receptor antagonists, such as e.g. VR-1 antagonists, glutamate receptor antagonists, such as e.g. mGlu5 receptor antagonists, mGlu1 receptor antagonists, iGlu5 receptor antagonists, AMPA receptor antagonists, purine receptor blockers, such as e.g. P2X3 antagonists, NO-synthase inhibitors, such as e.g. INOS inhibitors, calcium channel blockers, such as e.g. PQ-type blockers, N-type blockers, potassium channel openers, such as e.g. KCNQ channel openers, sodium channel blockers, such as e.g. PN3 channel blockers, NMDA receptor antagonists, acid-sensing ion channel antagonists, such as e.g. ASIC3 antagonists, bradykinin receptor antagonists such as e.g. B1 receptor antagonists, cannabinoid receptor agonists, such as e.g. CB2 agonists, CB1 agonists, somatostatin receptor agonists, such as e.g. sst2 receptor agonists may be added.

The dosage of these active substances is expediently 1/5 of the lowest usually recommended dose to 1/1 of the normally recommended dose, i.e. for example 20 to 100 mg of sumatriptan.

### Formulations

The compounds prepared according to the invention may be administered either on their own or optionally in combination with other active substances for the treatment of migraine by intravenous, subcutaneous, intramuscular, intraarticular, intrarectal, intranasal route, by inhalation, topically, transdermally or orally, while aerosol formulations are particularly suitable for inhalation. The combinations may be administered either simultaneously or sequentially.

Suitable forms for administration are for example tablets, capsules, solutions, syrups, emulsions or inhalable powders or aerosols. The content of the pharmaceutically effective compound(s) in each case should be in the range from 0.1 to 90 wt. %, preferably 0.5 to 50 wt. % of the total composition, i.e. In amounts which are sufficient to achieve the dosage range specified hereinafter.

The preparations may be administered orally in the form of a tablet, as a powder, as a powder in a capsule (e.g. a hard gelatine capsule), as a solution or suspension. When administered by inhalation the active substance combination may be given as a powder, as an aqueous or aqueous-ethanolic solution or using a propellant gas formulation.

Preferably, therefore, pharmaceutical formulations are characterised by the content of one or more compounds of formula I according to the preferred embodiments above.

It is particularly preferable if the compounds of formula I are administered orally, and it is also particularly preferable if they are administered once or twice a day. Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number of layers to achieve delayed release, possibly using the excipients mentioned above for the tablets.

Syrups containing the active substances or combinations thereof according to the invention may additionally contain a sweetener such as saccharine, cyclamate, glycerol or sugar and a flavour enhancer, e.g. a flavouring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Capsules containing one or more active substances or combinations of active substances may for example be prepared by mixing the active substances with inert carriers such as lactose or sorbitol and packing them into gelatine capsules. Suitable suppositories may be made for example by mixing with carriers provided for this purpose, such as neutral fats or polyethyleneglycol or the derivatives thereof.

Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose), emulsifiers (e.g. lignin, spent sulphite liquors, methylcellulose, starch and polyvinylpyrrolidone) and lubricants (e.g. magnesium stearate, talc, stearic acid and sodium lauryl sulphate).

For oral administration the tablets may, of course, contain, apart from the abovementioned carriers, additives such as sodium citrate, calcium carbonate and dicalcium phosphate together with various additives such as starch, preferably potato starch, gelatine and the like. Moreover, lubricants such as magnesium stearate, sodium lauryl sulphate and talc may be used at the same time for the tabletting process. In the case of aqueous suspensions the active substances may be combined with various flavour enhancers or colourings in addition to the excipients mentioned above.

It is also preferred if the compounds of formula I are administered by inhalation, particularly preferably if they are administered once or twice a day. For this purpose, the compounds of formula I have to be made available in forms suitable for inhalation. Inhalable preparations include inhalable powders, propellant-containing metered-dose aerosols or propellant-free inhalable solutions, which are optionally present in admixture with conventional physiologically acceptable excipients.

Within the scope of the present invention, the term propellant-free inhalable solutions also includes concentrates or sterile ready-to-use inhalable solutions. The preparations which may be used according to the invention are described in more detail in the next part of the specification.

### Inhalable Powders

If the compounds of formula I are present in admixture with physiologically acceptable excipients, the following physiologically acceptable excipients may be used to prepare the inhalable powders according to the invention: monosaccharides (e.g. glucose or arabinose), disaccharides (e.g. lactose, saccharose, maltose), oligo- and polysaccharides (e.g. dextrans), polyalcohols (e.g. sorbitol, mannitol, xylitol), salts (e.g. sodium chloride, calcium carbonate) or mixtures of these excipients with one another. Preferably, mono- or disaccharides are used, while the use of lactose or glucose is preferred, particularly, but not exclusively, in the form of their hydrates. For the purposes of the invention, lactose is the particularly preferred excipient, while lactose monohydrate is most particularly preferred. Methods of preparing the inhalable powders according to the invention by grinding and micronising and by finally mixing the components together are known from the prior art.

### Propellant-Containing Inhalable Aerosols

The propellant-containing inhalable aerosols which may be used according to the invention may contain I dissolved in the propellant gas or in dispersed form. The propellant gases which may be used to prepare the inhalation aerosols are known from the prior art. Suitable propellant gases are selected from among hydrocarbons such as n-propane, n-butane or isobutane and halohydrocarbons such as preferably fluorinated derivatives of methane, ethane, propane, butane, cyclopropane or cyclobutane. The propellant gases mentioned above may be used on their own or in mixtures thereof. Particularly preferred propellant gases are fluorinated alkane derivatives selected from TG134a (1,1,1,2-tetrafluoroethane), TG227 (1,1,1,2,3,3,3-heptafluoropropane) and mixtures thereof. The propellant-driven inhalation aerosols used within the scope of the use according to the invention may also contain other ingredients such as co-solvents, stabilisers, surfactants, antioxidants, lubricants and pH adjusters. All these ingredients are known in the art.

### Propellant-Free Inhalable Solutions

The compounds of formula I are preferably used according to the invention to prepare propellant-free inhalable solutions and inhalable suspensions. Solvents used for this purpose include aqueous or alcoholic, preferably ethanolic solutions. The solvent may be water on its own or a mixture of water and ethanol. The solutions or suspensions are adjusted to a pH of 2 to 7, preferably 2 to 5, using suitable acids. The pH may be adjusted using acids selected from inorganic or organic acids. Examples of particularly suitable inorganic acids include hydrochloric acid, hydrobromic acid, nitric acid, sulphuric acid and/or phosphoric acid. Examples of particularly suitable organic acids include ascorbic acid, citric acid, malic acid, tartaric acid, maleic acid, succinic acid, fumaric acid, acetic acid, formic acid and/or propionic acid etc. Preferred inorganic acids are hydrochloric and sulphuric acids. It is also possible to use the acids which have already formed an acid addition salt with one of the active substances. Of the organic acids, ascorbic acid, fumaric acid and citric acid are preferred. If desired, mixtures of the above acids may also be used, particularly in the case of acids which have other properties in addition to their acidifying qualities, e.g. as flavourings, antioxidants or complexing agents, such as citric acid or ascorbic acid, for example. According to the invention, it is particularly preferred to use hydrochloric acid to adjust the pH.

Co-solvents and/or other excipients may be added to the propellant-free inhalable solutions used for the purpose according to the invention. Preferred co-solvents are those which contain hydroxyl groups or other polar groups, e.g. alcohols—particularly isopropyl alcohol, glycols—particularly propyleneglycol, polyethyleneglycol, polypropyleneglycol, glycolether, glycerol, polyoxyethylene alcohols and polyoxyethylene fatty acid esters. The terms excipients and additives in this context denote any pharmacologically acceptable substance which is not an active substance but which can be formulated with the active substance or substances in the pharmacologically suitable solvent in order to improve the qualitative properties of the active substance formulation. Preferably, these substances have no pharmacological effect or, in connection with the desired therapy, no appreciable or at least no undesirable pharmacological effect. The excipients and additives include, for example, surfactants such as soya lecithin, oleic acid, sorbitan esters, such as polysorbates, polyvinylpyrrolidone, other stabilisers, complexing agents, antioxidants and/or preservatives which guarantee or prolong the shelf life of the finished pharmaceutical formulation, flavourings, vitamins and/or other additives known in the art. The additives also include pharmacologically acceptable salts such as, for example, sodium chloride as isotonic agents. The preferred excipients include antioxidants such as ascorbic acid, for example, provided that it has not already been used to adjust the pH, vitamin A, vitamin E, tocopherols and similar vitamins or provitamins occurring in the human body. Preservatives may be used to protect the formulation from contamination with pathogens. Suitable preservatives are those which are known in the art, particularly cetyl pyridinium chloride, benzalkonium chloride or benzoic acid or benzoates such as sodium benzoate in the concentration known from the prior art.

For the treatment forms described above, read-to-use packs of a medicament are provided for the treatment of respiratory complaints, containing an explanatory leaflet mentioning the words respiratory complaint, COPD or asthma, for example, a pteridine and one or more combination partners selected from the group mentioned above.

### EXPERIMENTAL SECTION

As a rule IR, ¹H-NMR and mass spectra have been obtained for the compounds prepared. Unless stated otherwise, R_{f} values are determined using ready-made TLC silica gel plates 60 F254 (E. Merck, Darmstadt, Item no. 1.05714) without chamber saturation.

The ratios given for the eluants relate to units by volume of the particular solvents. The units by volume given for NH₃ relate to a concentrated solution of NH₃ in water.

Unless stated otherwise, the acid, base and salt solutions used in working up the reaction solutions are aqueous systems of the specified concentrations. Silica gel made by Millipore (MATREX™, 35-70 μm) is used for chromatographic purifications.

The HPLC data provided are measured under the parameters listed below:

### Method A:

| | | percent by volume of |
|---|---|---|
| | percent by volume of water | acetonitrile |
| time (min) | (with 0.1% formic acid) | (with 0.1% formic acid) |
| 0 | 95 | 5 |
| 9 | 10 | 90 |
| 10 | 10 | 90 |
| 11 | 95 | 5 |

Analytical column: Zorbax column (Agilent Technologies), SB (Stable Bond) C18; 3.5 μm; 4.6×75 mm; column temperature: 30° C.; flow: 0.8 mL/min; injection volume: 5 μL; detection at 254 nm

### Method B:

| | | percent by volume of |
|---|---|---|
| | percent by volume of water | acetonitrile |
| time (min) | (with 0.1% formic acid) | (with 0.1% formic acid) |
| 0 | 95 | 5 |
| 4.5 | 10 | 90 |
| 5 | 10 | 90 |
| 5.5 | 90 | 10 |

Analytical column: Zorbax column (Agilent Technologies), SB (Stable Bond) C18; 3.5 μm; 4.6×75 mm; column temperature: 30° C.; flow: 1.6 mL/min; injection volume: 5 μL; detection at 254 nm

### Method C:

| | | percent by volume of |
|---|---|---|
| | percent by volume of water | acetonitrile |
| time (min) | (with 0.1% formic acid) | (with 0.1% formic acid) |
| 0 | 95 | 5 |
| 4.5 | 10 | 90 |
| 5 | 10 | 90 |
| 5.5 | 90 | 10 |

Analytical column: Zorbax column (Agilent Technologies), SB (Stable Bond) C18; 3.5 μm; 4.6×75 mm; column temperature: 30° C.; flow: 1.6 mL/min; injection volume: 5 μL; detection at 254 nm

Method D:

| | | percent by volume of |
|---|---|---|
| | percent by volume of water | acetonitrile |
| time (min) | (with 0.04% TFA) | (with 0.04% TFA) |
| 0 | 80 | 20 |
| 15 | 20 | 80 |
| 17 | 20 | 80 |

Analytical column: Symmetry C8 Waters−4.6×150 mm; 5 micron, flow: 1.3 ml/min, column temperature: 25° C., detection at 254 nm.

In preparative HPLC purifications as a rule the same gradients are used as were used to obtain the analytical HPLC data.

The products are collected under mass control, the fractions containing product are combined and freeze-dried.

In the absence of any more information regarding the configuration, it is unclear whether there are pure enantiomers involved or whether partial or even total racemisation has taken place.

The following abbreviations are used in the test descriptions:

Cyc cyclohexane
DCM dichloromethane
DIPE diisopropylether

### DMF N,N-dimethylformamide

EtOAc ethyl acetate
EtOH ethanol
AcOH acetic acid
i.vac. in vacuo (under vacuum)
MeOH methanol
MTBE tert-butylmethylether
NaOAc sodium acetate
RT room temperature
TBTU O-(benzotriazol-1-yl)-N,N,N′,N′-tetramethyluronium-tetrafluoroborate
THF tetrahydrofuran

### Component A1

### (R)-1-carboxy-2-(3-ethyl-4-hydroxy-5-methyl-phenyl)-ethyl 4-(2-oxo-1,2,4,5-tetrahydro-1,3-benzodiazepin-3-yl)-piperidine-1-carboxylate

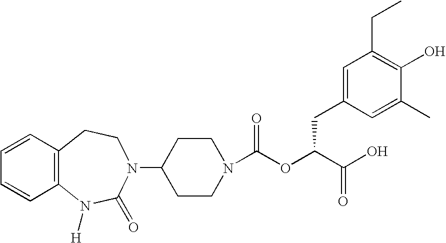

### A1a) 2-ethyl-6-methyl-phenol

At 0° C., 19.4 mL (0.23 mmol) concentrated HCl and a solution of 16.1 g (0.23 mmol) sodium nitrite in water (ca. 70 mL) were added to a solution of 30 g (222 mmol) 2-ethyl-6-methyl-aniline in 135 mL EtOH and stirred for 15 min. This mixture was added at 45° C. to a solution of 10.5 mL concentrated H₂SO₄ in 300 mL water and at the end of the addition heated to 70° C. The aqueous phase was cooled to RT and exhaustively extracted with EtOAc. The combined organic phases were extracted with 1 M NaOH solution. The aqueous phase was washed with DCM, acidified to pH 1 with 4 N HCl solution and extracted with DCM. The organic phase was washed with saturated NaCl solution, dried on Na₂SO₄ and concentrated by evaporation i. vac. The crude product was used in the next reaction step without further purification.

Yield: 12.0 g (40% of theory)

### A1) 4-bromo-2-ethyl-6-methyl-phenol

A solution of 12.7 mL (247 mmol) bromine in 10 mL chloroform was added dropwise at RT to a solution of 33.6 g (247 mmol) 2-ethyl-6-methyl-phenol in 350 mL chloroform and the mixture was stirred for 2 h. The reaction mixture was combined with an aqueous NaHSO₃ solution and stirred for 20 min. The phases were separated and the organic phase was washed with saturated NaCl solution, dried on Na₂SO₄ and concentrated by evaporation i. vac. Column chromatography (silica gel, Cyc/EtOAc 9:1) yielded the product.

Yield: 39.8 g (75% of theory)

ESI-MS: (M+H)⁺=214/216 (Br)

Retention time (HPLC-MS): 6.3 min (Method D)

### A1c) 2-benzyloxy-5-bromo-1-ethyl-3-methyl-benzene

A suspension of 39.8 g (185 mmol) 4-bromo-2-ethyl-6-methyl-phenol, 63.9 g (0.46 mmol) K₂CO₃ and 22.0 mL (185 mmol) benzylbromide in 450 mL acetonitrile was refluxed for 3 h, cooled to RT and concentrated by evaporation i. vac. The residue was combined with EtOAc, the organic phase was washed with water and saturated NaCl solution, dried on Na₂SO₄ and concentrated by evaporation i. vac.

Yield: 54.5 g (96% of theory)

ESI-MS: (M+H)⁺=304/306 (Br)

Retention time (HPLC-MS): 9.4 min (Method D)

### A1d) methyl (Z,E)-2-acetylamino-3-(4-benzyloxy-3-ethyl-5-methyl-phenyl)-acrylate

Prepared analogously to Example 1b from 50.4 g (165.1 mmol) 2-benzyloxy-5-bromo-1-ethyl-3-methyl-benzene and 28.9 g (198.2 mmol) methyl 2-acetylamino-acrylate.

Yield: 41.0 g (68% of theory)

ESI-MS: (M+H)⁺=368

Retention time (HPLC-MS): 4.5 min (Method C)

### A1e) 3-(4-benzyloxy-3-ethyl-5-methyl-phenyl)-2-oxo-propionic acid

200 mL of 4 M HCl were added to a solution of 41.0 g (111.6 mmol) methyl (Z,E)-2-acetylamino-3-(4-benzyloxy-3-ethyl-5-methyl-phenyl)-acrylate in 300 mL 1,4-dioxane and the reaction solution was heated to 130° C. (bath temperature) for 7 h. The organic phase was separated off while hot, concentrated by evaporation i. vac. and the residue obtained was recrystallised from toluene.

Yield: 9.6 g (28% of theory)

ESI-MS: (M+H)⁺=312

Retention time (HPLC-MS): 4.1 min (Method C)

### A1f) (R)-3-(4-benzyloxy-3-ethyl-5-methyl-phenyl)-2-hydroxy-propionic acid

Under an argon atmosphere a solution of 9.59 g (30.7 mmol) 3-(4-benzyloxy-3-ethyl-5-methyl-phenyl)-2-oxo-propionic acid in 25 mL THF was combined with 4.26 mL (31.0 mmol) triethylamine, the mixture was stirred for 5 min and cooled to −30° C. (internal temperature). A solution of 19.7 g (61.0 mmol) (IR)-B-chlorodiisopinocampheylborane in 35 mL was added dropwise and after the addition had ended the reaction solution was stirred for 30 min without cooling. It was combined with 15 mL of 4 N NaOH (temperature rise to 20° C.), stirred for 5 min, cooled to 0° C., combined with 50 mL MTBE and stirred for 20 min. The organic phase was separated off and dried on Na₂SO₄. After elimination of the desiccant and solvent the residue was reacted further without purification.

Yield: 10.3 g (100% of theory)

ESI-MS: (M−H)-=313

Retention time (HPLC-MS): 4.2 min (Method C)

### A1g) methyl (R)-3-(4-benzyloxy-3-ethyl-5-methyl-phenyl)-2-hydroxy-propionate

Prepared analogously to Example 1e from 10.3 g (30.7 mmol) (R)-3-(4-benzyloxy-3-ethyl-5-methyl-phenyl)-2-hydroxy-propionic acid and 4.71 mL (64.5 mmol) thionyl chloride. The crude product obtained was reacted further without purification.

### A1h) (R)-2-(4-benzyloxy-3-ethyl-5-methyl-phenyl)-1-methoxycarbonyl-ethyl 4-(2-oxo-1,2,4,5-tetrahydro-1,3-benzodiazepin-3-yl)-piperidine-1-carboxylate

Within 10 min a solution of 7.12 g (34.3 mmol) 4-nitrophenyl-chloroformate in 30 mL THF was added to a solution of 75 mL pyridine heated to 60° C. (bath temperature), the mixture was stirred for 10 min and then a solution of 10.0 g of the crude product from Example A1g in 50 mL pyridine was added dropwise. The mixture was stirred for another 1 h, combined with 6.72 g (27.4 mmol) 3-piperidin-4-yl-1,3,4,5-tetrahydro-1,3-benzodiazepin-2-one and the bath temperature was increased to 100° C. (2 h). The precipitate formed was filtered, the filtrate was concentrated by evaporation i. vac., the residue was combined with 150 mL EtOAc, the organic phase was washed twice with 50 mL of 1 M KHSO₄ solution and ten times with 50 mL of 15% K₂CO₃ solution and dried on Na₂SO₄. After elimination of the desiccant and solvent the residue was purified by chromatography (silica gel, EtOAc/Cyc 2:1).

Yield: 2.28 g (14% of theory)

ESI-MS: (M+H)⁺=600

Retention time (HPLC-MS): 5.4 min (Method C)

### A1i) (R)-2-(4-benzyloxy-3-ethyl-5-methyl-phenyl)-1-carboxy-ethyl 4-(2-oxo-1,2,4,5-tetrahydro-1,3-benzodiazepin-3-yl)-piperidine-1-carboxylate

A solution of 50 mg (2.09 mmol) LiOH in 5 mL water was added to a solution of 800 mg (1.33 mmol) (R)-2-(4-benzyloxy-3-ethyl-5-methyl-phenyl)-1-methoxy-carbonyl-ethyl 4-(2-oxo-1,2,4,5-tetrahydro-1,3-benzodiazepin-3-yl)-piperidine-1-carboxylate in 15 mL THF and the reaction mixture was stirred for 1 h at RT. It was evaporated down i.vac., the residue was taken up in 50 mL water and combined with 2 M HCl until an acid reaction was obtained. The precipitate formed was filtered off, washed with water and dried. Further purification was carried out by decocting with 150 mL water, filtration and drying again.

Yield: quantitative

ESI-MS: (M+H)⁺=586

Retention time (HPLC-MS): 4.8 min (Method C)

### A1k) (R)-1-carboxy-2-(3-ethyl-4-hydroxy-5-methyl-phenyl)-ethyl 4-(2-oxo-1,2,4,5-tetrahydro-1,3-benzodiazepin-3-yl)-piperidine-1-carboxylate

810 mg (1.38 mmol) (R)-2-(4-benzyloxy-3-ethyl-5-methyl-phenyl)-1-carboxy-ethyl 4-(2-oxo-1,2,4,5-tetrahydro-1,3-benzodiazepin-3-yl)-piperidine-1-carboxylate in 25 mL MeOH were combined with 80 mg 10% Pd/C and hydrogenated at RT and 3 bar hydrogen until the reaction stopped. The catalyst was removed by suction filtering and the solvent was concentrated by evaporation i. vac. The residue was triturated with DIPE, suction filtered and dried.

Yield: 639 mg (93% of theory)

ESI-MS: (M+H)⁺=496

Retention time (HPLC-MS): 3.7 min (Method C)

### Component A2

### (R)-1-carboxy-2-(4-hydroxy-3-methoxy-5-methyl-phenyl)-ethyl 4-(2-oxo-1,2,4,5-tetrahydro-1,3-benzodiazepin-3-yl)-piperidine-1-carboxylate

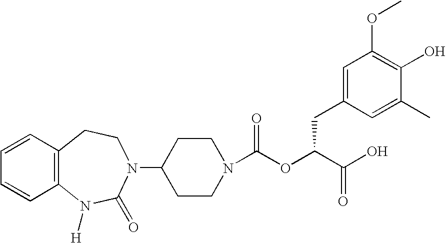

### A2a) 4-bromo-2-methoxy-6-methyl-phenol

Within 5.5 h a solution of 56.2 g (0.32 mol) N-bromosuccinimide in 1700 mL AcOH was added dropwise to a solution of 42.3 g (0.31 mol) 2-methoxy-6-methyl-phenol in 450 mL AcOH and the mixture was stirred for 16 h at RT. The reaction mixture was concentrated by evaporation i. vac. and the residue was taken up in DCM. The organic phase was washed with 5% NaHCO₃ and saturated NaCl solution, dried on Na₂SO₄ and concentrated by evaporation i. vac. The red oil was used in the next reaction step without further purification.

Yield: 65.9 g (66% of theory)

R_{f}=0.32 (silica gel, hexane/EtOAc 4:1)

Retention time (HPLC-MS): 11.1 min (Method D)

### A2b) 2-benzyloxy-5-bromo-1-methoxy-3-methyl-benzene

At RT, 45.7 g (0.33 mol) K₂CO₃ and a solution of 40.3 mL (0.33 mol) benzylbromide were added to a solution of 65.9 g (0.26 mol) 4-bromo-2-methoxy-6-methyl-phenol in 330 mL DMF and the mixture was stirred for 18 h at RT. The mixture was filtered, concentrated by evaporation i. vac. and the residue was taken up in diethyl ether. The organic phase was washed with water, 5% Na₂CO₃ and NaCl solution, dried on Na₂SO₄ and concentrated by evaporation i. vac. The crude product was used in the next reaction step without further purification.

Yield: 92.2 g (81% of theory)

R_{f}=0.56 (silica gel, hexane/EtOAc 4:1)

Retention time (HPLC-MS): 16.3 min (Method D)

A2c) 4-benzyloxy-3-methoxy-5-methyl-benzaldehyde

At −75° C., 96 mL (240 mmol) n-butyllithium (2.5 M in hexane) were added dropwise to a solution of 61.2 g (119.5 mmol) 2-benzyloxy-5-bromo-1-methoxy-3-methyl-benzene in 240 mL THF and the mixture was stirred for 15 min at −75° C. A solution of 31 mL (402 mmol) DMF in 30 mL THF was added dropwise, the mixture was heated to 0° C. and stirred for a further 2 h. The reaction was combined with saturated NH₄Cl solution, diluted with 150 mL water and the phases were separated. The aqueous phase was exhaustively extracted with diethyl ether. The combined organic phases were washed with saturated NaCl solution, dried on Na₂SO₄ and concentrated by evaporation i. vac. Column chromatography (silica gel, hexane/EtOAc 85:15) yielded the product as a yellow oil.

Yield: 27.1 g (88% of theory)

R_{f}=0.32 (silica gel, hexane/EtOAc 4:1)

Retention time (HPLC-MS): 13.3 min (Method D)

### A2d) 2-acetylamino-3-(4-benzyloxy-3-methoxy-5-methyl-phenyl)-acrylic acid

A suspension of 27.0 g (105.4 mmol) 4-benzyloxy-3-methoxy-5-methyl-benzaldehyde, 18.5 g (158.0 mmol) N-acetylglycine and 12.96 g (158.0 mmol) NaOAc in 120 mL acetic anhydride was heated to 115° C. under nitrogen for 3.5 h. At 100° C., 60 mL of water were slowly added dropwise and the mixture was stirred for 1 h. The reaction mixture was cooled to RT, poured into water and the aqueous phase was exhaustively extracted with EtOAc. The combined organic phases were washed with saturated NaCl solution, dried on Na₂SO₄ and concentrated by evaporation i. vac. The residue was triturated with isopropanol, the solid obtained was washed with isopropanol, diethyl ether and a little acetone and dried at 45° C. i.vac.

Yield: 21.2 (57% of theory)

R_{f}=0.24 (silica gel, hexane/EtOAc 4:1)

Retention time (HPLC-MS): 9.4 min (Method D)

### A2e) 3-(4-benzyloxy-3-methoxy-5-methyl-phenyl)-2-oxo-propionic acid

The product was obtained analogously to Example 1c starting from 20.0 g (56.3 mmol) 2-acetylamino-3-(4-benzyloxy-3-methoxy-5-methyl-phenyl)-acrylic acid. The crude product was used in the next reaction step without further purification.

Yield: 15.6 g (53% of theory)

Retention time (HPLC-MS): 11.9 min (Method D)

### A2f) (R)-3-(4-benzyloxy-3-methoxy-5-methyl-phenyl)-2-hydroxy-propionic acid

The product was prepared analogously to Example 1d starting from 16.0 g (50.90 mmol) 3-(4-benzyloxy-3-methoxy-5-methyl-phenyl)-2-oxo-propionic acid.

Yield: 7.63 g (47% of theory)

Retention time (HPLC-MS): 9.8 min (Method D)

### A2g) methyl (R)-3-(4-benzyloxy-3-methoxy-5-methyl-phenyl)-2-hydroxy-propionate

The product was prepared analogously to Example 1e starting from 7.6 g (24.02 mmol) (R)-3-(4-benzyloxy-3-methoxy-5-methyl-phenyl)-2-hydroxy-propionic acid.

Yield: 6.84 g (86% of theory)

Retention time (HPLC-MS): 11.7 min (Method D)

### A2h) (R)-2-(4-benzyloxy-3-methoxy-5-methyl-phenyl)-1-methoxycarbonyl-ethyl 4-(2-oxo-1,2,4,5-tetrahydro-1,3-benzodiazepin-3-yl)-piperidine-1-carboxylate

The product was prepared analogously to Example 1f starting from 6.8 g (20.6 mmol) methyl (R)-3-(4-benzyloxy-3-methoxy-5-methyl-phenyl)-2-hydroxy-propionate in acetonitrile.

Yield: 8.16 g (66% of theory)

ESI-MS: (M+H)⁺=602

Retention time (HPLC-MS): 14.1 min (Method D)

### A2i) (R)-2-(4-benzyloxy-3-methoxy-5-methyl-phenyl)-1-carboxy-ethyl 4-(2-oxo-1,2,4,5-tetrahydro-1,3-benzodiazepin-3-yl)-piperidine-1-carboxylate

The product was prepared analogously to Example 1g starting from 8.16 g (13.65 mmol) (R)-2-(4-benzyloxy-3-methoxy-5-methyl-phenyl)-1-methoxycarbonyl-ethyl 4-(2-oxo-1,2,4,5-tetrahydro-1,3-benzodiazepin-3-yl)-piperidine-1-carboxylate.

Yield: 7.83 g (98% of theory)

ESI-MS: (M+H)⁺=588

Retention time (HPLC-MS): 12.2 min (Method D)

### A2 k) (R)-1-carboxy-2-(4-hydroxy-3-methoxy-5-methyl-phenyl)-ethyl 4-(2-oxo-1,2,45-tetrahydro-1,3-benzodiazepin-3-yl)-piperidine-1-carboxylate

The product was prepared analogously to Example 1h starting from 7.80 g (13.27 mmol) (R)-2-(4-benzyloxy-3-methoxy-5-methyl-phenyl)-1-carboxy-ethyl 4-(2-oxo-1,2,4,5-tetrahydro-1,3-benzodiazepin-3-yl)-piperidine-1-carboxylate.

Yield: 5.33 g (80% of theory)

ESI-MS: (M+H)⁺=498

Retention time (HPLC-MS): 8.4 min (Method D)

### Example 1

### (R)-2-[4-(4-ethoxycarbonyl-phenyl)-piperazin-1-yl]-1-(4-hydroxy-3,5-dimethyl-benzyl)-2-oxo-ethyl 4-(2-oxo-1,2,4,5-tetrahydro-1,3-benzodiazepin-3-yl)-piperidine-1-carboxylate

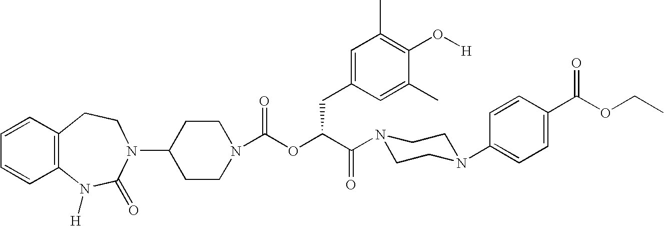

### 1a) 2-benzyloxy-5-bromo-1,3-dimethylbenzene

39.9 g (286 mmol) K₂CO₃ were added to a solution of 50.0 g (249 mmol) 2,6-dimethyl-4-bromophenol in 500 mL DMF and the mixture was stirred for 20 min. Then 34.0 mL (286 mmol) benzyl chloride were slowly added dropwise and the reaction mixture was stirred for 3 h at 100° C. bath temperature. After the reaction had ended the mixture was poured onto 500 mL water and exhaustively extracted with EtOAc. The organic phases were combined, dried on Na₂SO₄ and concentrated by evaporation i. vac.

Yield: quantitative

GC-MS: (M⁺)=290/292 (Br)

R_{f}=0.87 (silica gel, Cyc/EtOAc 3:1)

### 1b) methyl 2-acetylamino-3-(4-benzyloxy-3,5-dimethyl-phenyl)-acrylate

Under a nitrogen atmosphere a mixture of 40.0 g (137 mmol) 2-benzyloxy-5-bromo-1,3-dimethylbenzene and 24.1 g (165 mmol) methyl 2-acetylamino-acrylate in 420 mL triethylamine and 200 mL acetonitrile was combined with 3.5 g (11.2 mmol) tri-o-tolyl-phosphane and 2.5 g (11.1 mmol) Pd(OAc)₂ and stirred for 18 h at 80° C. The precipitate was suction filtered, the filtrate was concentrated by evaporation i. vac. and combined with 800 mL DCM and 800 mL water. The organic phase was separated off, dried on Na₂SO₄, suction filtered, the solvent was eliminated i.vac., the residue was stirred with EtOAc, suction filtered and dried i. vac.

Yield: 31.1 g (64% of theory)

ESI-MS: (M+H)⁺=354

Retention time (HPLC-MS): 8.6 min (Method A)

### 1c) 3-(4-benzyloxy-3,5-dimethyl-phenyl)-2-oxo-propionic acid

31.1 g (88.1 mmol) methyl 2-acetylamino-3-(4-benzyloxy-3,5-dimethyl-phenyl)-acrylate in 150 mL of 1,4-dioxane were combined with 125 mL of 4 M HCl, refluxed for 7 h and stirred overnight at RT. The precipitate was suction filtered, washed with water and dried at 45° C. in the vacuum drying cupboard.

Yield: 14.3 g (54% of theory)

EI-MS: (M)⁺=298

Retention time (HPLC-MS): 9.0 min (Method A)

### 1d) (R)-3-(4-benzyl-3,5-dimethyl-phenyl)-2-hydroxy-propionic acid

Under a nitrogen atmosphere a solution of 14.3 g (47.8 mmol) 3-(4-benzyloxy-3,5-dimethyl-phenyl)-2-oxo-propionic acid and 8.3 mL (59.8 mmol) triethylamine in 170 mL THF was combined at −35° C. with a solution of 22.1 (69.0 mmol) (IR)-B-chlorodiisopinocampheylboran in 70 mL THF within 30 min. After the addition had ended the cooling bath was removed and the reaction solution was stirred overnight at RT. The reaction mixture was made alkaline at 0° C. with 70 mL 1 M NaOH, combined with 100 mL MTBE, stirred for 15 min and the phases were separated. The organic phase was washed with 50 mL water and three times with 50 mL of 1 M NaOH. The combined aqueous phases were acidified with semiconc. HCl, exhaustively extracted with EtOAc and the combined organic phases were dried on Na₂SO₄. After elimination of the desiccant and solvent the residue was reacted further without purification.

Yield: 14.0 g (98% of theory)

ESI-MS: (M−H)-=299

Retention time (HPLC-MS): 7.9 min (Method A)

### 1e) methyl (R)-3-(4-benzyloxy-3,5-dimethyl-phenyl)-2-hydroxy-propionate

2.0 mL (27.4 mmol) SOCl₂ were added dropwise to a solution, cooled to 0° C., of 14.0 g (23.3 mmol) (R)-3-(4-benzoyl-3,5-dimethyl-phenyl)-2-hydroxy-propionic acid in 150 mL MeOH and the reaction mixture was stirred for 1 h at RT. The reaction solution was evaporated down i. vac. and the residue was purified by chromatography (silica gel, Cyc/EtOAc 3:1).

Yield: 5.7 g (78% of theory)

ESI-MS: (M+NH₄)⁺=332

Retention time (HPLC-MS): 9.1 min (Method A)

### 1f) (R)-2-(4-benzyloxy-3,5-dimethyl-phenyl)-1-methoxycarbonyl-ethyl 4-(2-oxo-1,2,4,5-tetrahydro-1,3-benzodiazepin-3-yl)-piperidine-1-carboxylate

Under a nitrogen atmosphere 1.93 g (9.58 mmol) 4-nitrophenyl chloroformate was added to a solution of 1.17 g (9.58 mmol) 4-dimethylaminopyridine in 50 mL pyridine, the mixture was stirred for 1.5 h at RT, combined with 3.0 g (9.58 mmol) methyl (R)-3-(4-benzyloxy-3,5-dimethyl-phenyl)-2-hydroxy-propionate and stirred for 20 min at RT. Then 2.35 g (9.58 mmol) 3-piperidin-4-yl-1,3,4,5-tetrahydro-1,3-benzodiazepin-2-one were added and the mixture was stirred for 20 h at RT. The reaction mixture was concentrated by evaporation i. vac., the residue was taken up in EtOAc, the organic phase was washed with 10% KHSO₄ and saturated NaHCO₃ solution and dried on Na₂SO₄. After elimination of the desiccant and solvent the residue was purified by chromatography (silica gel, gradient Cyc/EtOAc 1:1 to 1:2).

Yield: 3.21 g (57% of theory)

ESI-MS: (M+H)⁺=586

Retention time (HPLC-MS): 10.4 min (Method A)

### 1g) (R)-2-(4-benzyloxy-3,5-dimethyl-phenyl)-1-carboxy-ethyl 4-(2-oxo-1,2,4,5-tetrahydro-1,3-benzodiazepin-3-yl)-piperidine-1-carboxylate

A solution of 3.21 g (5.48 mmol) (R)-2-(4-benzyloxy-3,5-dimethyl-phenyl)-1-methoxy-carbonyl-ethyl 4-(2-oxo-1,2,4,5-tetrahydro-1,3-benzodiazepin-3-yl)-piperidine-1-carboxylate in 80 mL THF was combined with a solution of 200 mg (8.35 mmol) LiOH in 40 mL of water and stirred for 1 h at RT. The reaction mixture was concentrated by evaporation i. vac., the residue was taken up in 100 mL water, acidified with 2 M HCl, the precipitate was suction filtered and dried at 40° C. in the vacuum drying cupboard.

Yield: quantitative

ESI-MS: (M+H)⁺=572

Retention time (HPLC-MS): 9.2 min (Method A)

### 1h) (R)-2-(4-hydroxy-3,5-dimethyl-phenyl)-1-carboxy-ethyl 4-(2-oxo-1,2,4,5-tetrahydro-1,3-benzodiazepin-3-yl)-piperidine-1-carboxylate

3.72 g (6.51 mmol) (R)-2-(4-benzyloxy-3,5-dimethyl-phenyl)-1-carboxy-ethyl 4-(2-oxo-1,2,4,5-tetrahydro-1,3-benzodiazepin-3-yl)-piperidine-1-carboxylate in 50 mL DCM were combined with 300 mg 10% Pd/C and shaken at RT and 3 bar hydrogen until the reaction stopped. The catalyst was removed by suction filtering and the solvent was concentrated by evaporation i. vac. The residue was triturated with DIPE and suction filtered.

Yield: 2.41 g (77% of theory)

ESI-MS: (M+H)⁺=482

Retention time (HPLC-MS): 7.0 min (Method A)

### 1i) (R)-2-[4-(4-ethoxycarbonyl-phenyl)-piperazin-1-yl]-1-(4-hydroxy-3,5-dimethyl-benzyl)-2-oxo-ethyl 4-(2-oxo-1,2,4,5-tetrahydro-1,3-benzodiazepin-3-yl)-piperidine-1-carboxylate

A solution of 200 mg (0.42 mmol) (R)-2-(4-hydroxy-3,5-dimethyl-phenyl)-1-carboxy-ethyl 4-(2-oxo-1,2,4,5-tetrahydro-1,3-benzodiazepin-3-yl)-piperidine-1-carboxylate, 148 mg (0.46 mmol) TBTU and 65 μL (0.46 mmol) triethylamine in 2 mL DMF was stirred for 10 min at RT. Then 108 mg (0.46 mmol) ethyl 4-piperazin-1-yl-benzoate was added and the reaction solution was stirred for 2 h. The reaction solution was purified by HPLC without further working up; the fractions containing the product were combined and lyophilised.

Yield: 216 mg (75% of theory)

ESI-MS: (M+H)⁺=698

Retention time (HPLC-MS): 4.4 min (Method B)

### Example 1.1

### (R)-2-[4-(4-carboxy-phenyl)-piperazin-1-yl]-1-(4-hydroxy-3,5-dimethyl-benzyl)-2-oxo-ethyl 4-(2-oxo-1,2,4,5-tetrahydro-1,3-benzodiazepin-3-yl)-piperidine-1-carboxylate

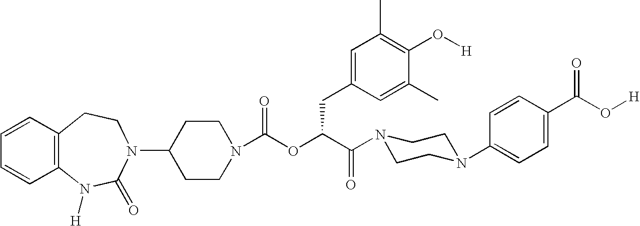

A solution of 2.6 mg (0.11 mmol) LiOH in 1 mL water was added to a solution of 50 mg (0.07 mmol) (R)-2-[4-(4-ethoxycarbonyl-phenyl)-piperazin-1-yl]-1-(4-hydroxy-3,5-dimethyl-benzyl)-2-oxo-ethyl 4-(2-oxo-1,2,4,5-tetrahydro-1,3-benzodiazepin-3-yl)-piperidine-1-carboxylate in 2 mL THF and the reaction mixture was shaken overnight. To complete the reaction 6.5 μL (0.08 mmol, 35% in water) H₂O₂ were added and shaken for another 4 h at RT. It was evaporated down i.vac., the residue was taken up in water and acidified with formic acid. The precipitate formed was suction filtered and dried.

Yield: 37 mg (77% of theory)

ESI-MS: (M+H)⁺=670

Retention time (HPLC-MS): 4.0 min (Method B)

### Example 2

### (R)-1-(4-amino-3-chloro-5-trifluoromethyl-benzyl)-2-[4-(4-ethoxycarbonyl-phenyl)-piperazin-1-yl]-2-oxo-ethyl 4-(2-oxo-1,2,4,5-tetrahydro-1,3-benzodiazepin-3-yl)-piperidine-1-carboxylate

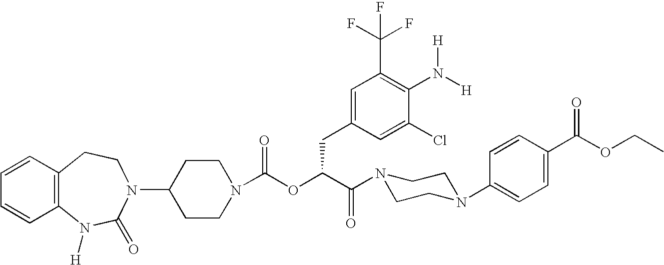

### 2a) (Z,E)-2-acetylamino-3-(4-amino-3-chloro-5-trifluoromethyl-phenyl)-acrylic acid

39.7 g (335 mmol) N-acetylglycine were added to a suspension of 50.0 g (224 mmol) 4-amino-3-chloro-5-trifluoromethyl-benzaldehyde and 27.5 g (335 mmol) NaOAc in 202 mL acetic anhydride and the reaction mixture was heated to 115° C. for 1 h. After cooling to 80° C. 100 mL water were added dropwise, while the temperature of the mixture was kept at 80° C. The suspension was again heated to 95° C. for 40 min and then added to a mixture of 250 mL toluene and 500 mL water. The suspension was stirred at RT, the precipitate was suction filtered and dried at 60° C. in the circulating air dryer.

Yield: 48.8 g (68% of theory)

ESI-MS: (M+H)⁺=321/323 (Cl)

R_{f}=0.37 (silica gel, DCM/MeOH/AcOH 90:10:1)

### 2b) 3-(4-amino-3-chloro-5-trifluoromethyl-phenyl)-2-oxo-propionic acid

A suspension of 97.0 g (300 mmol) (Z,E)-2-acetylamino-3-(4-amino-3-chloro-5-tri-fluoromethyl-phenyl)-acrylic acid in 900 mL 1,4-dioxane and 1050 mL of 4 M HCl was heated to 100° C. for 8 h. The mixture was evaporated down i.vac. To about 600 mL, cooled to RT, the substance precipitated was filtered off, washed with twice 100 mL water and dried at 50° C. The residue was taken up in 850 mL toluene, refluxed and then cooled in the ice bath. The precipitate formed was filtered, washed with PE and dried in the circulating air dryer at 50° C.

Yield: 63.0 g (74% of theory)

ESI-MS: (M−H)-=280/282 (Cl)

R_{f}=0.21 (silica gel, DCM/MeOH/NH₃ 80:20:2)

### 2c) (R)-3-(4-amino-3-chloro-5-trifluoromethyl-phenyl)-2-hydroxy-propionic acid

A solution of 100.0 g (312 mmol) (IR)-B-chlorodiisopinocampheylboran in 150 mL THF was added dropwise to a solution, cooled to about −30° C., of 63.0 g (224 mmol) 3-(4-amino-3-chloro-5-trifluoromethyl-phenyl)-2-oxo-propionic acid and 31.2 mL (224 mmol) triethylamine in 300 mL THF and the reaction mixture was kept for 1.5 h at this temperature and then heated to RT within another hour. 80 mL of 4 M NaOH were added to the reaction mixture, it was stirred for 5 min, cooled to 0° C., combined with 300 mL MTBE, stirred again for 20 min at this temperature and then the phases were separated. The organic phase was exhaustively extracted with water, the combined aqueous phases were acidified with 4 M HCl, exhaustively extracted with MTBE and the combined organic phases were dried on Na₂SO₄. The THF/MTBE/NaOH phase was acidified with 4 M HCl, the phases were separated and the organic phase was concentrated by evaporation i. vac. The two residues were combined and reacted further without purification.

R_{f}=0.20 (silica gel, DCM/MeOH/NH₃ 80:20:2)

### 2d) methyl (R)-3-(4-amino-3-chloro-5-trifluoromethyl-phenyl)-2-hydroxy-propionate

The crude product from Example 2c (62 g) was dissolved in 300 mL MeOH and 3.65 mL (50 mmol) of SOCl₂ were slowly added dropwise to this solution. The reaction mixture was stirred for a further 3 h at RT, then concentrated by evaporation i. vac., the residue was taken up in DCM and filtered through silica gel. The solution was concentrated by evaporation i. vac. and the residue was purified by chromatography (silica gel, DCM/MeOH/NH₃ 80:20:2). The fractions containing the product were combined, concentrated by evaporation i. vac., the residue was combined with PE, suction filtered and dried.

Yield: 43.1 g (65% of theory over 2 steps)

ESI-MS: (M+H)⁺=298/300 (Cl)

R_{f}=0.86 (silica gel, DCM/MeOH/NH₃ 80:20:2)

### 2e) (R)-2-(4-amino-3-chloro-5-trifluoromethyl-phenyl)-1-methoxycarbonyl-ethyl 4-(2-oxo-1,2,4,5-tetrahydro-1,3-benzodiazepin-3-yl)-piperidine-1-carboxylate

Under a nitrogen atmosphere a solution of 13.5 g (65.0 mmol) 4-nitrophenyl chloroformate in 40 mL THF was metered into 100 mL pyridine at 60° C. (bath temperature) within 10 min, the mixture was stirred for 10 min, then a solution of 18.0 g (60.5 mmol) methyl (R)-3-(4-amino-3-chloro-5-trifluoromethyl-phenyl)-2-hydroxy-propionate in 50 ml of pyridine was added dropwise and the reaction mixture was kept 1.5 h at this temperature. Then 15.9 g (65.0 mmol) 3-piperidin-4-yl-1,3,4,5-tetrahydro-1,3-benzodiazepin-2-one was added batchwise. The temperature of the reaction mixture was increased to 100° C., kept for 6 h at this temperature and then stirred overnight at RT. It was evaporated down i.vac., the residue was taken up in 200 mL EtOAc, the organic phase was washed twice with 100 mL 1 M KHSO₄ solution, ten times with 50 mL of 15% K₂CO₃ solution and dried on Na₂SO₄. After elimination of the desiccant and solvent the residue was reacted further without purification.

Yield: 33.1 g (96% of theory)

ESI-MS: (M+H)⁺=569/571 (Cl)

R_{f}=0.72 (silica gel, DCM/Cyc/MeOH/NH₃ 70:15:15:2)

### 2f) (R)-2-(4-amino-3-chloro-5-trifluoromethyl-phenyl)-1-carboxy-ethyl 4-(2-oxo-1,2,4,5-tetrahydro-1,3-benzodiazepin-3-yl)-piperidine-1-carboxylate

A solution of 2.11 g (88.0 mmol) LiOH in 100 mL water was added to a solution of 33.0 g (58.0 mmol) (R)-2-(4-amino-3-chloro-5-trifluoromethyl-phenyl)-1-methoxy-carbonyl-ethyl 4-(2-oxo-1,2,4,5-tetrahydro-1,3-benzodiazepin-3-yl)-piperidine-1-carboxylate in 200 mL THF and the reaction solution was stirred for 3.5 h at RT. THF was eliminated i.vac., the aqueous residue was washed twice with MTBE, acidified with 2 M HCl, extracted exhaustively with DCM and the combined organic phases were dried on Na₂SO₄. After elimination of the desiccant and solvent the residue was dissolved at 65° C. in 80 mL isopropanol and slowly cooled to RT overnight. The suspension was cooled in the ice bath, suction filtered, washed with a little isopropanol and DIPE and dried.

Yield: 26.2 g (81% of theory)

ESI-MS: (M+H)⁺=555/557 (Cl)

R_{f}=0.18 (silica gel, DCM/Cyc/MeOH/NH₃ 70:15:15:2)

Retention time (HPLC): 4.0 min (Method B)

### 2g) (R)-1-(4-amino-3-chloro-5-trifluoromethyl-benzyl)-2-[4-(4-ethoxycarbonyl-phenyl)-piperazin-1-yl]-2-oxo-ethyl 4-(2-oxo-1,2,4,5-tetrahydro-1,3-benzodiazepin-3-yl)-piperidine-1-carboxylate

A solution of 200 mg (0.36 mmol) (R)-2-(4-amino-3-chloro-5-trifluoromethyl-phenyl)-1-carboxy-ethyl 4-(2-oxo-1,2,4,5-tetrahydro-1,3-benzodiazepin-3-yl)-piperidine-1-carboxylate, 128 mg (0.40 mmol) TBTU and 56 μL (0.40 mmol) triethylamine in 2 mL DMF was stirred for 10 min at RT. Then 94 mg (0.40 mmol) ethyl 4-piperazin-1-yl-benzoate were added and the reaction solution was stirred for 2 h. The reaction solution was purified by HPLC without further working up; the fractions containing the product were combined and lyophilised.

Yield: 265 mg (95% of theory)

ESI-MS: (M+H)⁺=771/773 (Cl)

Retention time (HPLC): 4.8 min (Method B)

The Examples that follow describe the preparation of pharmaceutical formulations which contain as active substance any desired compound of general formula I:

### Example I

### Capsules for Powder Inhalation Containing 1 mg of Active Ingredient

### Composition:

1 capsule for powder inhalation contains:

| | |
|---|---|
| active ingredient | 1.0 mg |
| lactose | 20.0 mg |
| hard gelatine capsules | 50.0 mg |
| | 71.0 mg |

### Method of Preparation:

The active ingredient is ground to the particle size required for inhaled substances. The ground active ingredient is homogeneously mixed with the lactose. The mixture is transferred into hard gelatine capsules.

### Example II

### Inhalable Solution for Respimat® Containing 1 mg of Active Ingredient

### Composition:

1 puff contains:

| | | |
|---|---|---|
| active ingredient | 1.0 | mg |
| benzalkonium chloride | 0.002 | mg |
| disodium edetate | 0.0075 | mg |
| purified water ad | 15.0 | μl |

### Method of Preparation:

The active ingredient and benzalkonium chloride are dissolved in water and transferred into Respimat® cartridges.

### Example III

### Inhalable Solution for Nebulisers Containing 1 Mg of Active Ingredient

### Composition:

1 vial contains:

| | | |
|---|---|---|
| active ingredient | 0.1 | g |
| sodium chloride | 0.18 | g |
| benzalkonium chloride | 0.002 | g |
| purified water ad | 20.0 | ml |

### Method of Preparation:

The active ingredient, sodium chloride and benzalkonium chloride are dissolved in water.

### Example IV

### Propellant Gas-Operated Metered Dose Aerosol Containing 1 Mg of Active Ingredient

### Composition:

1 puff contains:

| | |
|---|---|
| active ingredient | 1.0 mg |
| lecithin | 0.1% |
| propellant gas ad | 50.0 μl |

### Method of Preparation:

The micronised active ingredient is homogeneously suspended in the mixture of lecithin and propellant gas. The suspension is transferred into a pressurised container with a metering valve.

### Example V

### Nasal Spray Containing 1 mg of Active Ingredient

### Composition:

| | | |
|---|---|---|
| active ingredient | 1.0 | mg |
| sodium chloride | 0.9 | mg |
| benzalkonium chloride | 0.025 | mg |
| disodium edetate | 0.05 | mg |
| purified water ad | 0.1 | ml |

### Method of Preparation:

The active ingredient and the excipients are dissolved in water and transferred into a suitable container.

### Example VI

### Injectable Solution Containing 5 Mg of Active Substance Per 5 ml

### Composition:

| | | |
|---|---|---|
| active substance | 5 | mg |
| glucose | 250 | mg |
| human serum albumin | 10 | mg |
| glycofurol | 250 | mg |
| water for injections ad | 5 | ml |

### Preparation:

Glycofurol and glucose are dissolved in water for injections (WfI); human serum albumin is added; active ingredient is dissolved with heating; made up to specified volume with WfI; transferred into ampoules under nitrogen gas.

### Example VII

### Injectable Solution Containing 100 mg of Active Substance Per 20 Ml

### Composition:

| | | |
|---|---|---|
| active substance | 100 | mg |
| monopotassium dihydrogen phosphate = KH₂PO₄ | 12 | mg |
| disodium hydrogen phosphate = Na₂HPO₄*2H₂O | 2 | mg |
| sodium chloride | 180 | mg |
| human serum albumin | 50 | mg |
| Polysorbate 80 | 20 | mg |
| water for injections ad | 20 | ml |

### Preparation:

Polysorbate 80, sodium chloride, monopotassium dihydrogen phosphate and disodium hydrogen phosphate are dissolved in water for injections (WfI); human serum albumin is added; active ingredient is dissolved with heating; made up to specified volume with WfI; transferred into ampoules.

### Example VIII

Lyophilisate Containing 10 mg of Active Substance

Composition:

| | | |
|---|---|---|
| Active substance | 10 | mg |
| Mannitol | 300 | mg |
| human serum albumin | 20 | mg |
| water for injections ad | 2 | ml |

### Preparation:

Mannitol is dissolved in water for injections (WfI); human serum albumin is added; active ingredient is dissolved with heating; made up to specified volume with WfI; transferred into vials; freeze-dried.

### Solvent for Lyophilisate:

| | |
|---|---|
| Polysorbate 80 = Tween 80 | 20 mg |
| mannitol | 200 mg |
| water for injections ad | 10 ml |

### Preparation:

Polysorbate 80 and mannitol are dissolved in water for injections (WfI); transferred into ampoules.

### Example IX

### Tablets Containing 20 mg of Active Substance

### Composition:

| | |
|---|---|
| active substance | 20 mg |
| lactose | 120 mg |
| corn starch | 40 mg |
| magnesium stearate | 2 mg |
| Povidone K 25 | 18 mg |

### Preparation:

Active substance, lactose and corn starch are homogeneously mixed; granulated with an aqueous solution of Povidone; mixed with magnesium stearate; compressed in a tablet press; weight of tablet 200 mg.

### Example X

### Capsules Containing 20 mg Active Substance

### Composition:

| | |
|---|---|
| active substance | 20 mg |
| corn starch | 80 mg |
| highly dispersed silica | 5 mg |
| magnesium stearate | 2.5 mg |

### Preparation:

Active substance, corn starch and silica are homogeneously mixed; mixed with magnesium stearate; the mixture is packed into size for 3 hard gelatine capsules in a capsule filling machine.

### Example XI

### Suppositories Containing 50 mg of Active Substance

### Composition:

| | |
|---|---|
| active substance | 50 mg |
| hard fat (Adeps solidus) q.s. ad | 1700 mg |

### Preparation:

Hard fat is melted at about 38° C.; ground active substance is homogeneously dispersed in the molten hard fat; after cooling to about 35° C. it is poured into chilled moulds.

### Example XII

### Injectable Solution Containing 10 mg of Active Substance Per 1 ml

### Composition:

| | |
|---|---|
| active substance | 10 mg |
| mannitol | 50 mg |
| human serum albumin | 10 mg |
| water for injections ad | 1 ml |

### Preparation:

Mannitol is dissolved in water for injections (WfI); human serum albumin is added; active ingredient is dissolved with heating; made up to specified volume with WfI; transferred into ampoules under nitrogen gas.

1. A compound of the formula I 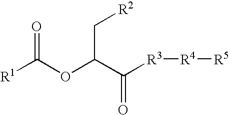
wherein
R¹ denotes a group selected from 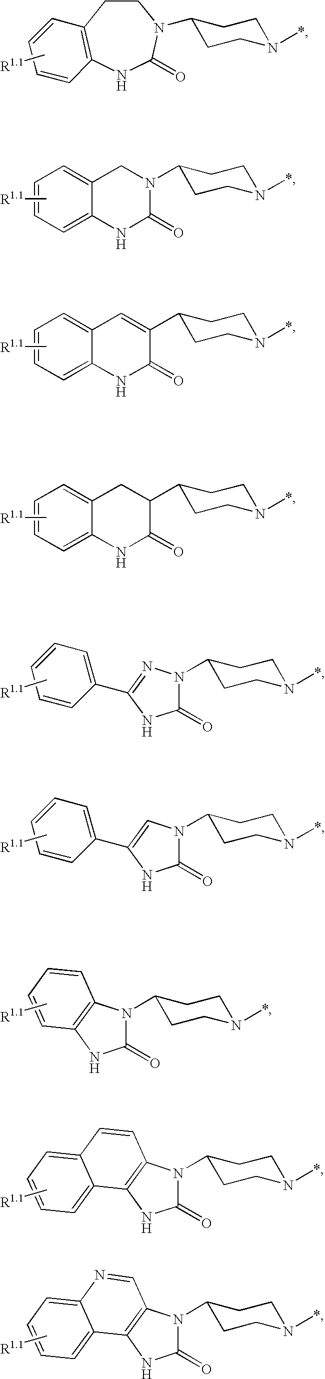 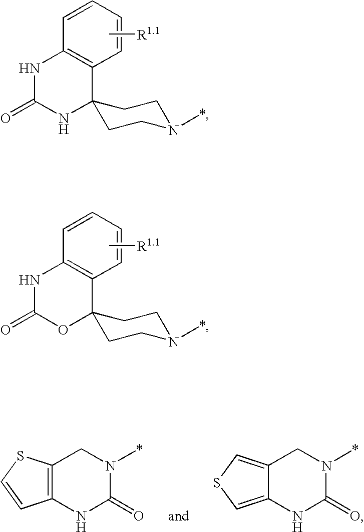
wherein
R^{1.1} denotes H, halogen, HO, F₃C or C₁₋₆-alkyl-O—,
R² denotes a group of general formulae II 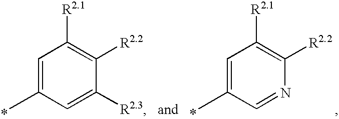
wherein
R^{2.1} denotes H, halogen, C₁₋₃-alkyl-O, C₁₋₃-alkyl or F₃C,
R^{2.2} denotes H, H₂N, HO, H₃C—O, H—C(O)—O or C₁₋₃-alkyl-C(O)—O,
R^{2.3} denotes H, halogen, C₁₋₃-alkyl or F₃C—,
R³ denotes a group of general formulae III 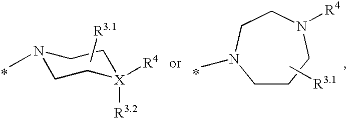
wherein
X denotes N or C,
R^{3.1} denotes H, C₁₋₃-alkyl or R^{3.1.1}—(O)C,
R^{3.1.1} denotes HO or C₁₋₆-alkyl-O,
R^{3.2} denotes a pair of free electrons, if X═N, or
R^{3.2} denotes H or C₁₋₃-alkyl-, if X═C,
R⁴ denotes a group selected from 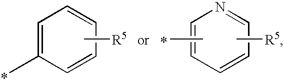
R⁵ denotes R^{5.1}—O—C(O)
R^{5.1} denotes H, C₁₋₈-alkyl, phenyl, indanyl, pyridyl-C₁₋₃-alkylene, HO—C₂₋₄-alkylene, C₁₋₆-alkyl-O—C₂₋₄-alkylene, HO—C₂₋₄-alkylene-O—C₂₋₄-alkylene, C₁₋₆-alkyl-C₂₋₄-alkylene-O—C₂₋₄-alkylene, H₂N—C₂₋₄-alkylene, (C₁₋₆-alkyl)-NH—C₂₋₄-alkylene, (C₁₋₆-alkyl)₂N—C₂₋₄-alkylene, H₂N—C(O)—C₁₋₃-alkylene, (C₁₋₆-alkyl)-NH—C(O)—C₁₋₃-alkylene, (C₁₋₆-alkyl)₂N—C(O)—C₁₋₃-alkylene, C₁₋₆-alkyl-C(O)—O—C₁₋₃-alkylene, C₁₋₆-alkyl-O—C(O)—O—C₁₋₃-alkylene, R^{5.1.1}—C(O)—C₁₋₃-alkylene or
R^{5.1.2}—C₂₋₄-alkylene,
R^{5.1.1} denotes a group selected from 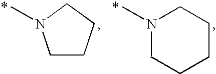
R^{5.1.2} denotes a group selected from 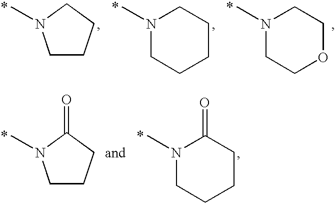
or a tautomer or salt thereof.

2. A compound of the formula I according to claim 1, wherein
R¹ denotes a group selected from 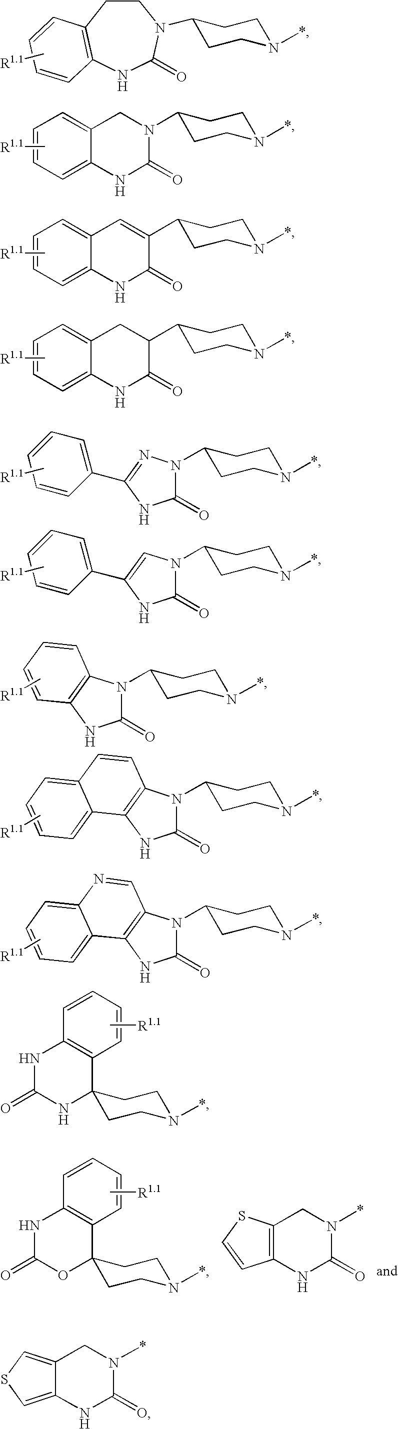
wherein
R^{1.1} denotes H or H₃C—O—,
R² denotes a group selected from 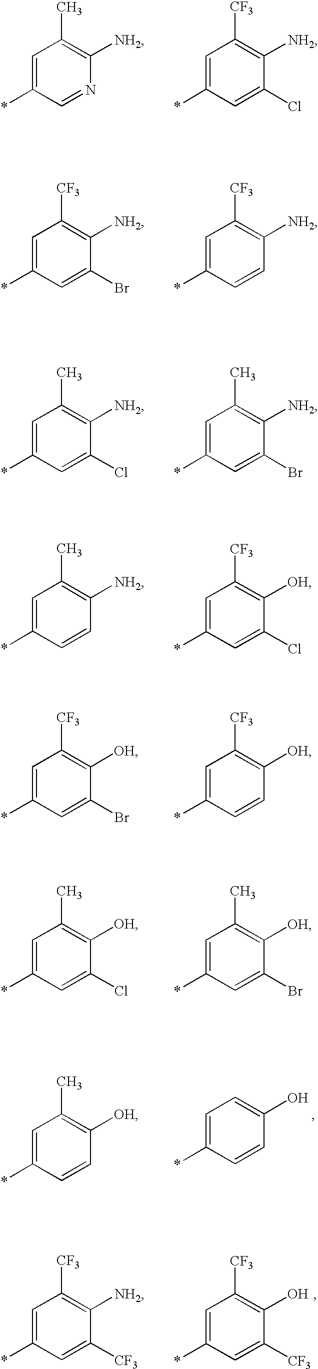 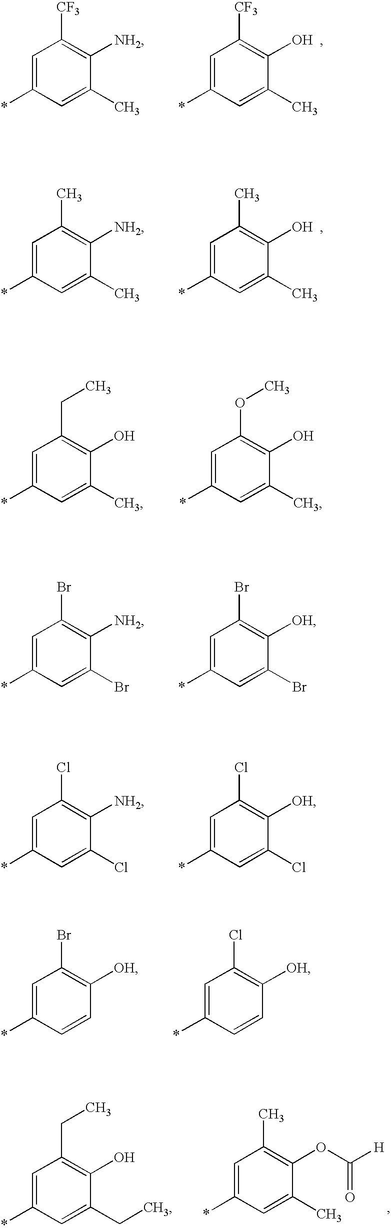 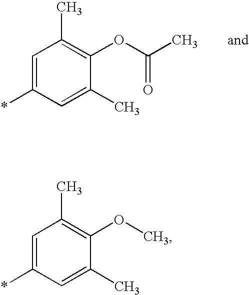
R³-R⁴ together denote a group selected from 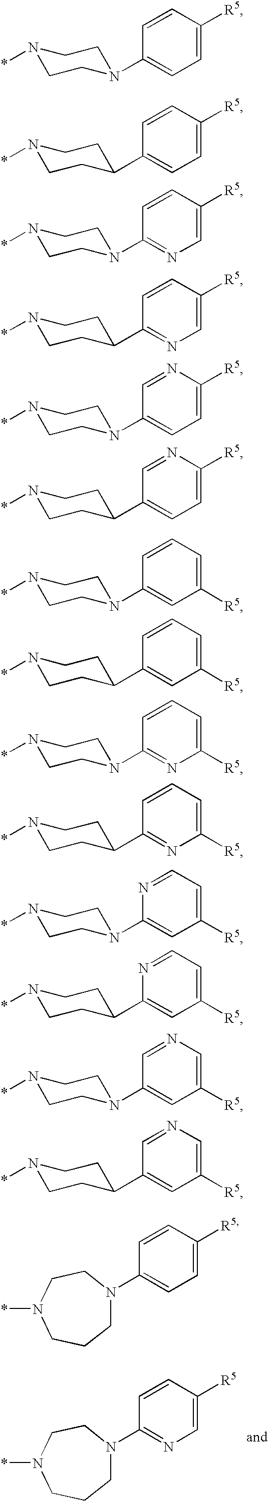 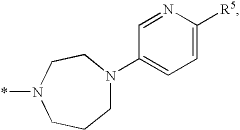
R⁵ denotes R⁵¹—O—C(O)— and
R^{5.1} denotes H, C₁₋₈-alkyl, phenyl, indanyl, pyridyl-C₁₋₃-alkylene, HO—C₂₋₄-alkylene, C₁₋₆-alkyl-O—C₂₋₄-alkylene, HO—C₂₋₄-alkylene-O—C₂₋₄-alkylene, C₁₋₆-alkyl-C₂₋₄-alkylene-O—C₂₋₄-alkylene, H₂N—C₂₋₄-alkylene, (C₁₋₆-alkyl)-NH—C₂₋₄-alkylene, (C₁₋₆-alkyl)₂N—C₂₋₄-alkylene, H₂N—C(O)—C₁₋₃-alkylene, (C₁₋₆-alkyl)-NH—C(O)—C₁₋₃-alkylene, (C₁₋₆-alkyl)₂N—C(O)—C₁₋₃-alkylene, C₁₋₆-alkyl-C(O)—O—C₁₋₃-alkylene, C₁₋₆-alkyl-O—C(O)—O—C₁₋₃-alkylene, R^{5.1.1}—C(O)—C₁₋₃-alkylene or R^{5.1.2}—C₂₋₄-alkylene,
R^{5.1.1} denotes a group selected from 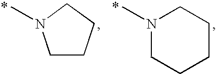
R^{5.1.2} denotes a group selected from 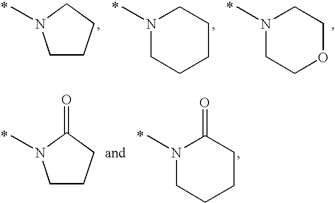
or a tautomer or salt thereof.

3. A compound of the formula I according to claim 1, wherein
R¹ denotes a group selected from 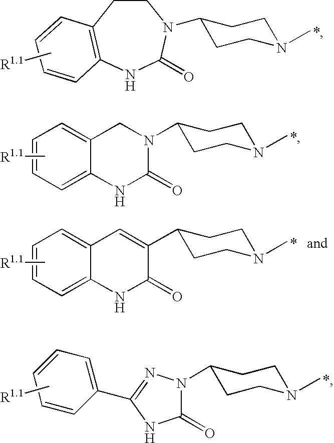
R² denotes a group selected from 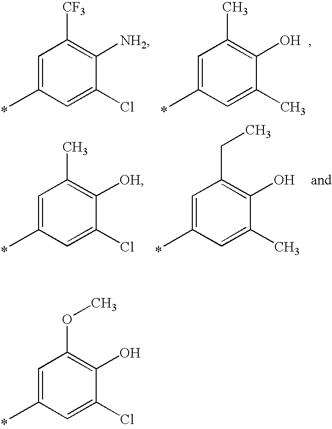
R³-R⁴ together denote a group selected from 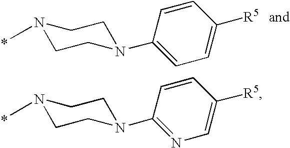
R⁵ denotes R^{5.1}—O—C(O)— and
R^{5.1} denotes H, C₁₋₆-alkyl, phenyl, indanyl, pyridyl-CH₂, C₁₋₃-alkyl-O—C₂₋₄-alkylene, C₁₋₃-alkyl-O—C₂₋₄-alkylene-O—C₂₋₄-alkylene, (C₁₋₃-alkyl)₂N—C₂₋₄-alkylene, (C₁₋₃-alkyl)₂N—C(O)—C₁₋₃-alkylene, C₁₋₆-alkyl-C(O)—O—C₁₋₃-alkylene, C₁₋₃-alkyl-O—C(O)—O—C₁₋₃-alkylene, R^{5.1.1}—C(O)—C₁₋₃-alkylene or R^{5.1.2}—C₂₋₄-alkylene,
R^{5.1.1} denotes a group selected from 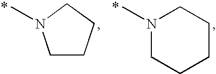
R^{5.1.2} denotes a group selected from 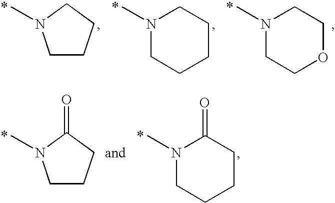
or a tautomer or salt thereof.

4. A compound of the formula I according to claim 1, selected from the group consisting of:

| No. | Structure |
|---|---|
| (1) | 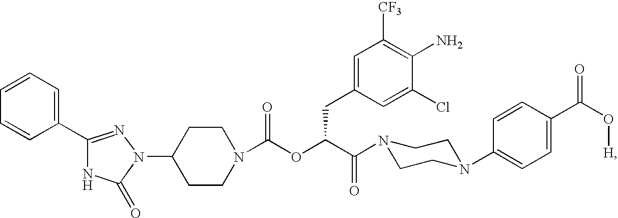 |
| (2) | 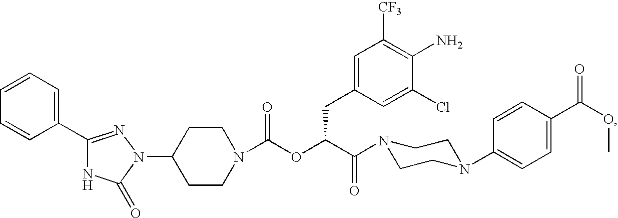 |
| (3) | 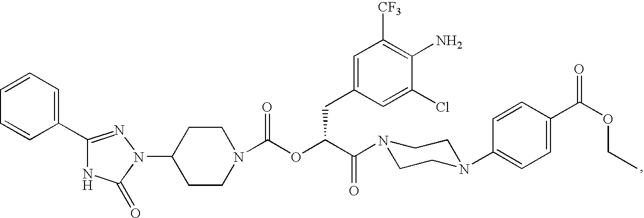 |
| (4) | 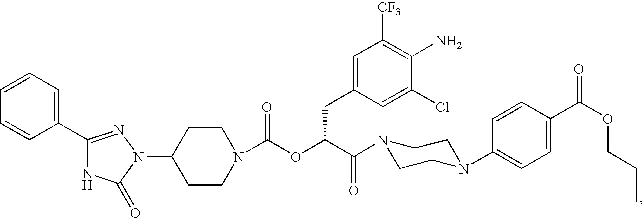 |
| (5) | 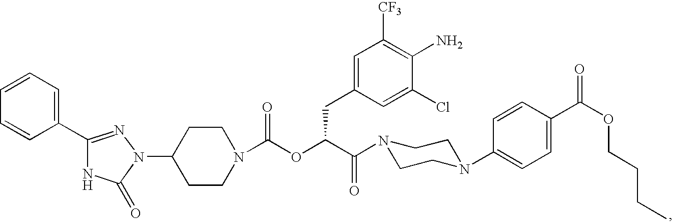 |
| (6) | 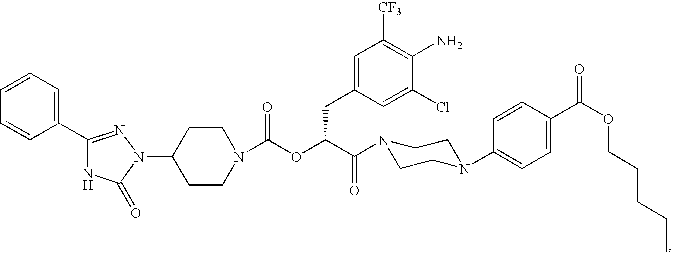 |
| (7) | 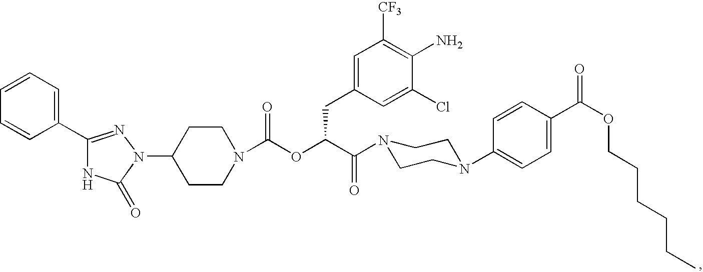 |
| (8) | 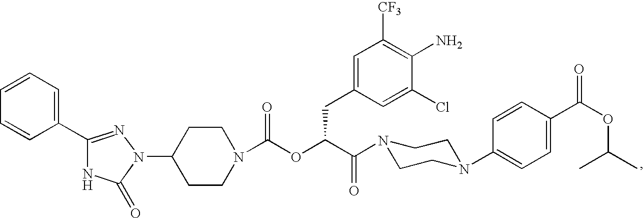 |
| (9) | 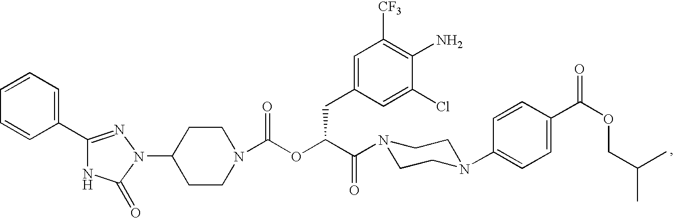 |
| (10) | 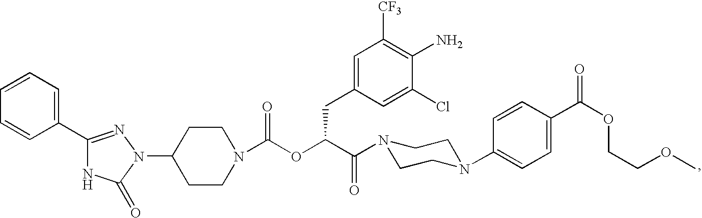 |
| (11) | 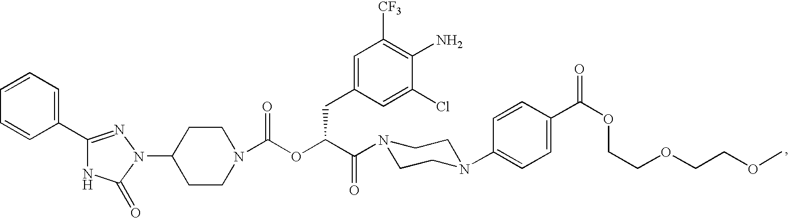 |
| (12) | 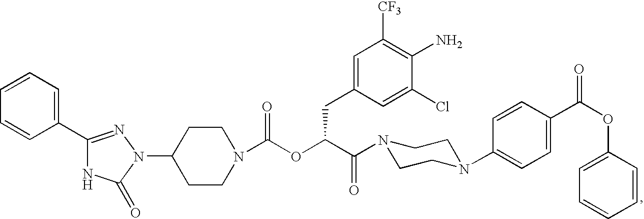 |
| (13) | 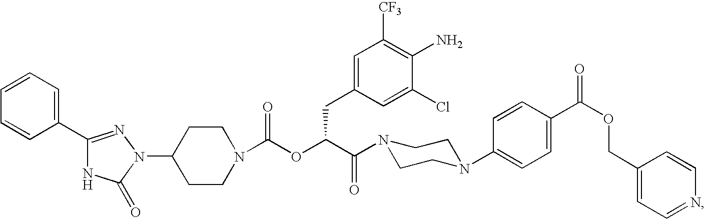 |
| (14) | 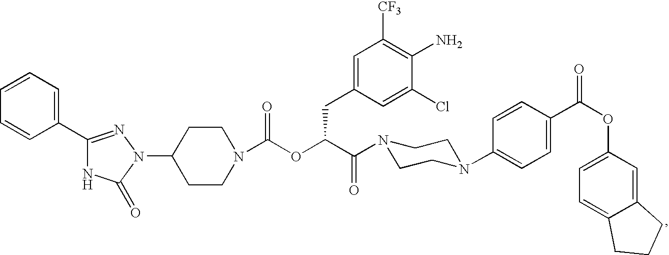 |
| (15) | 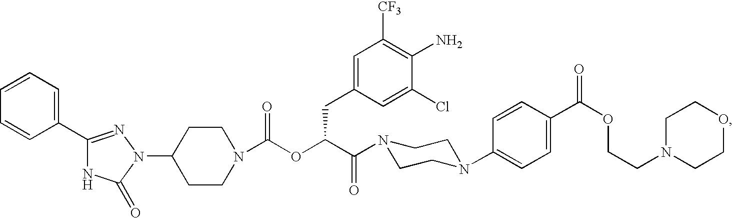 |
| (16) | 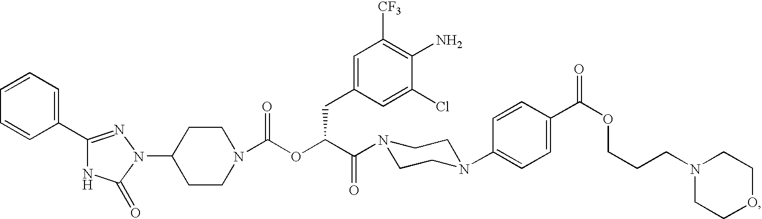 |
| (17) | 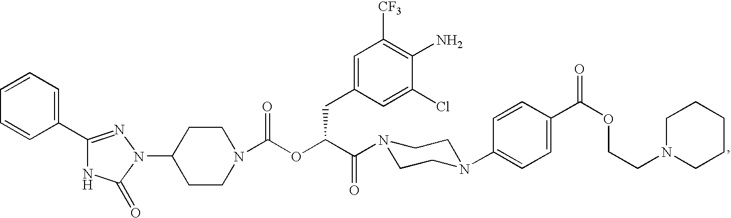 |
| (18) | 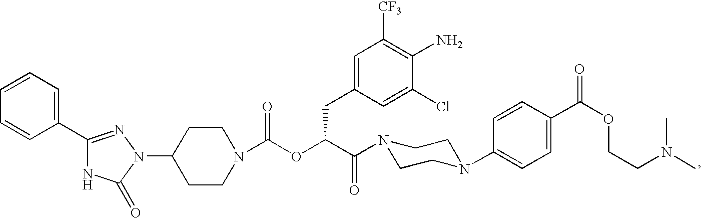 |
| (19) | 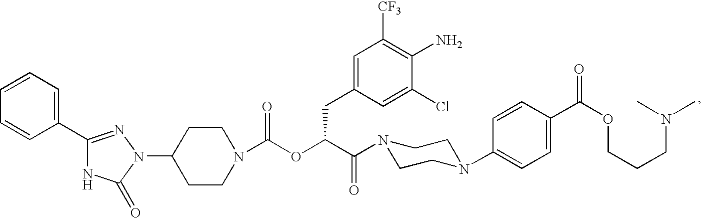 |
| (20) | 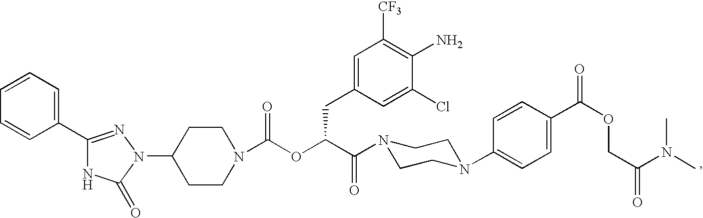 |
| (21) | 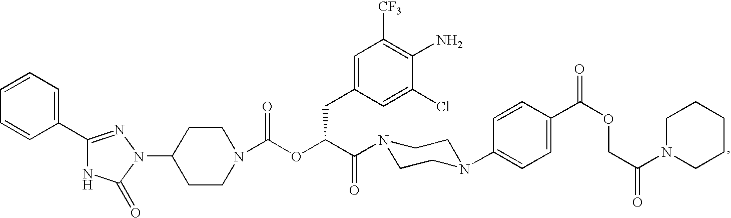 |
| (22) | 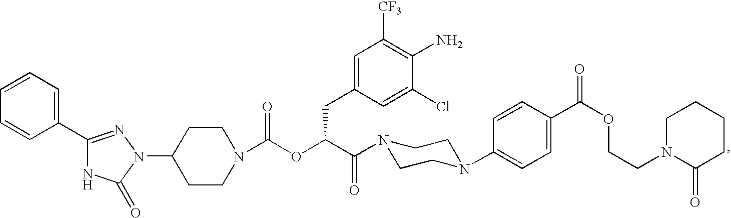 |
| (23) | 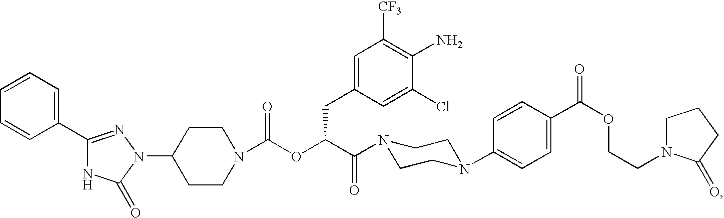 |
| (24) | 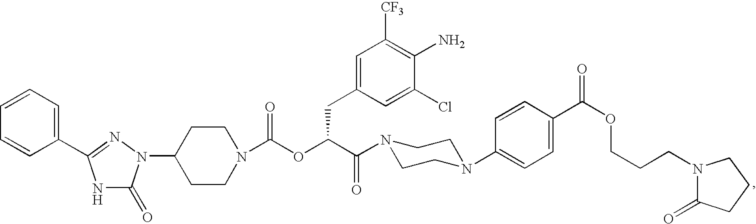 |
| (25) | 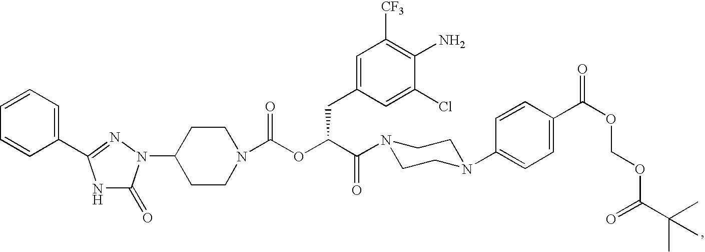 |
| (26) | 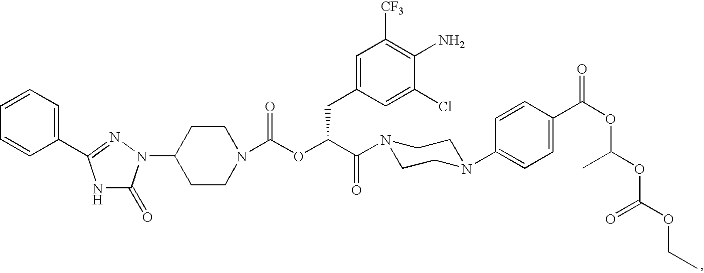 |
| (27) | 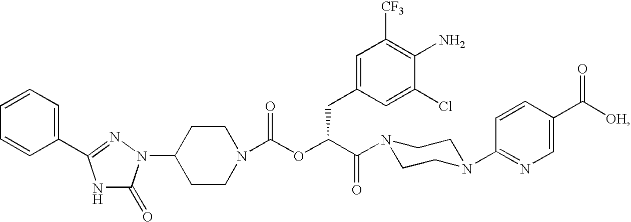 |
| (28) | 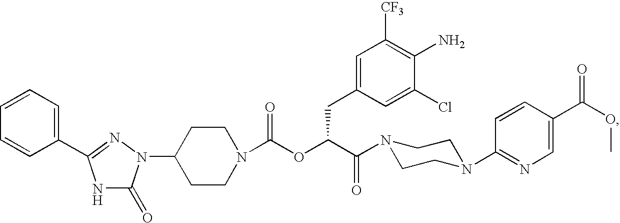 |
| (29) | 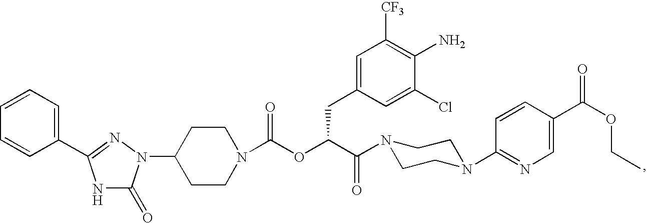 |
| (30) | 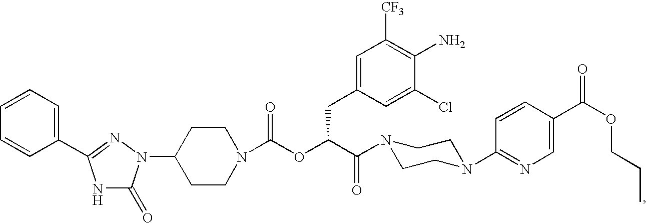 |
| (31) | 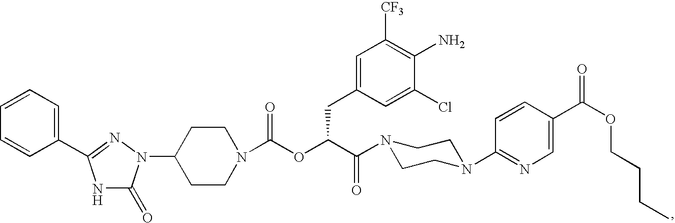 |
| (32) | 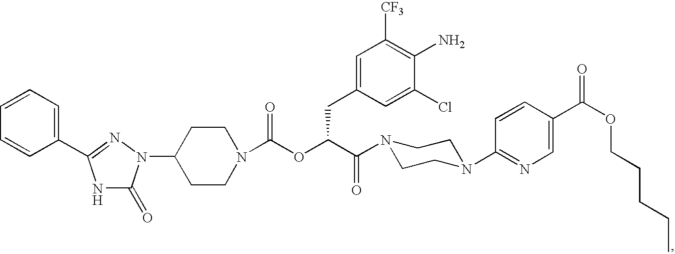 |
| (33) | 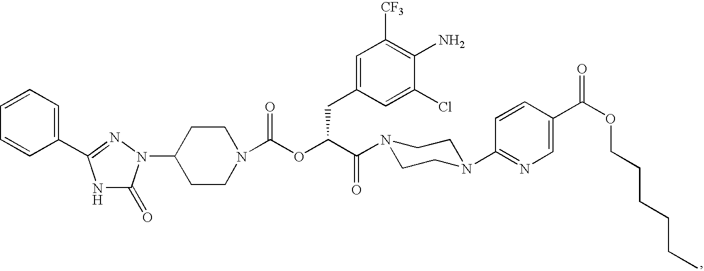 |
| (34) | 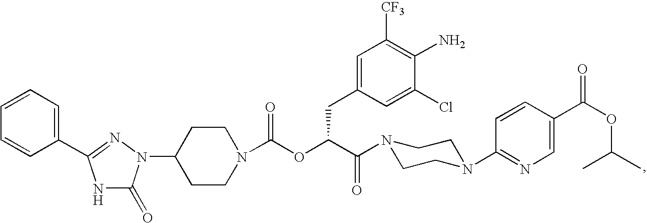 |
| (35) | 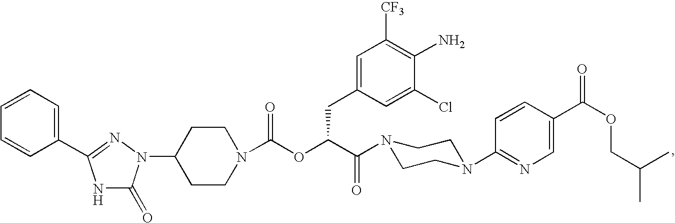 |
| (36) | 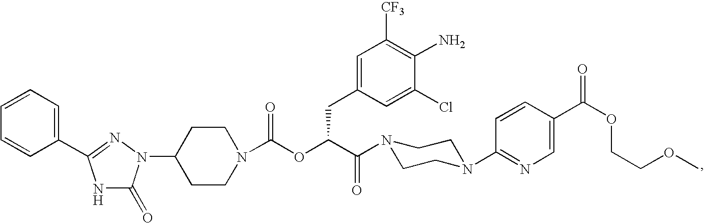 |
| (37) | 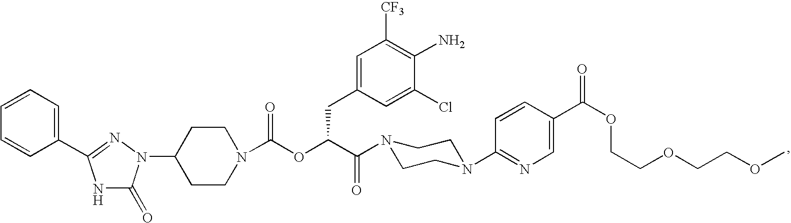 |
| (38) | 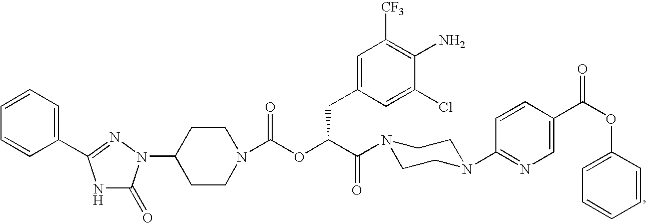 |
| (39) | 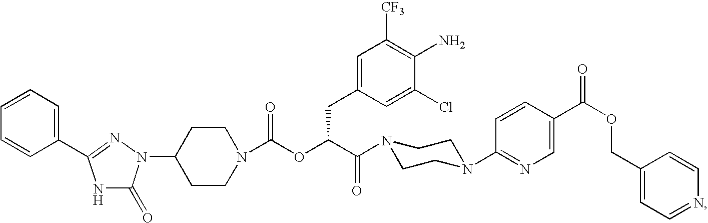 |
| (40) | 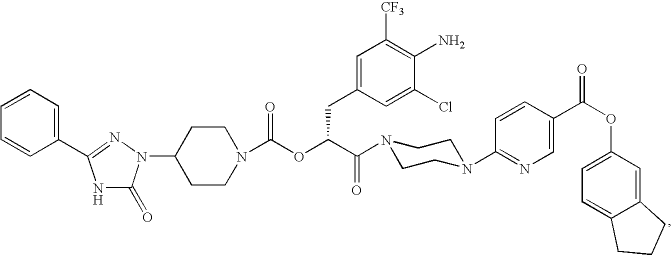 |
| (41) | 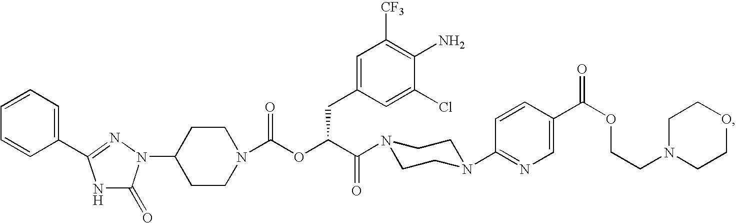 |
| (42) | 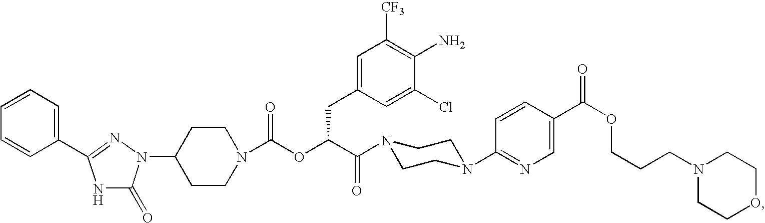 |
| (43) | 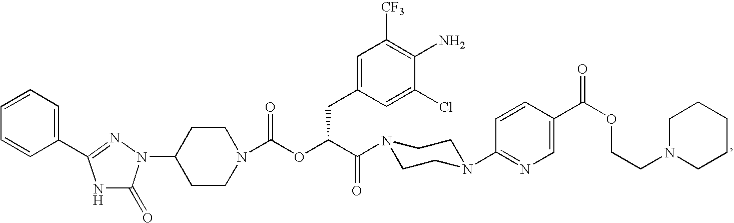 |
| (44) | 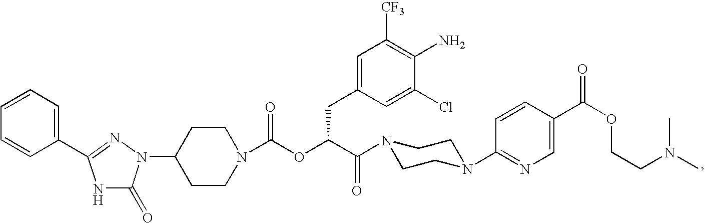 |
| (45) | 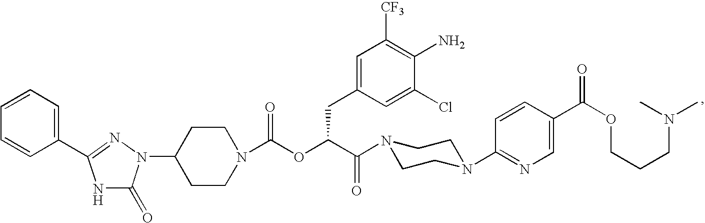 |
| (46) | 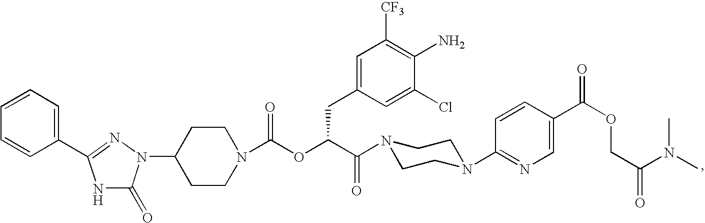 |
| (47) | 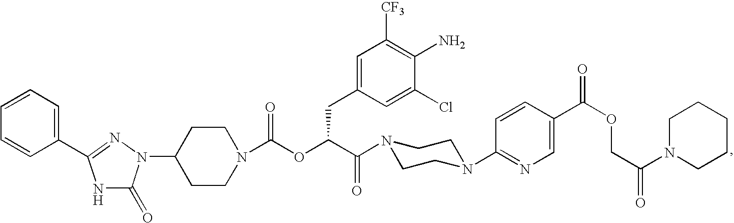 |
| (48) | 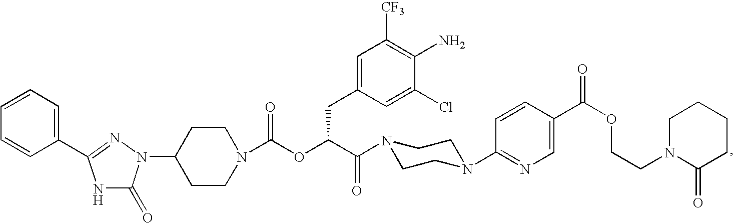 |
| (49) | 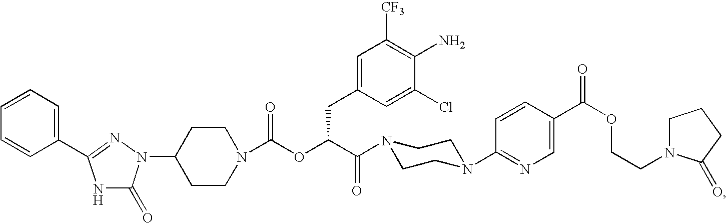 |
| (50) | 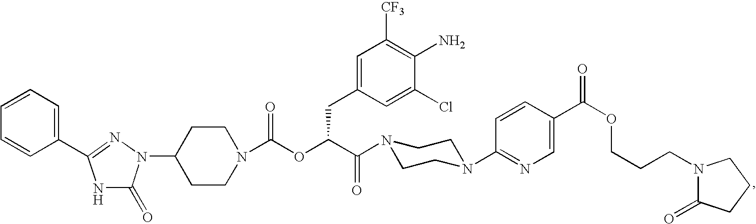 |
| (51) | 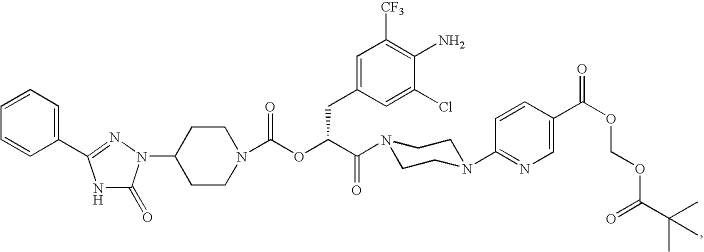 |
| (52) | 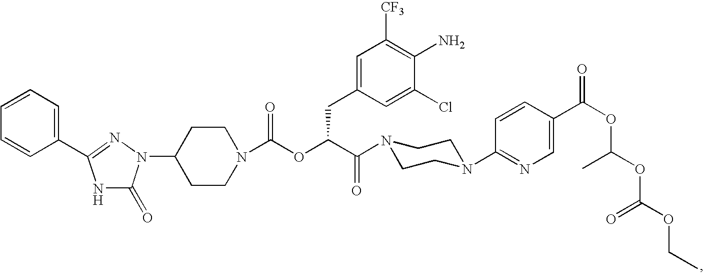 |
| (53) | 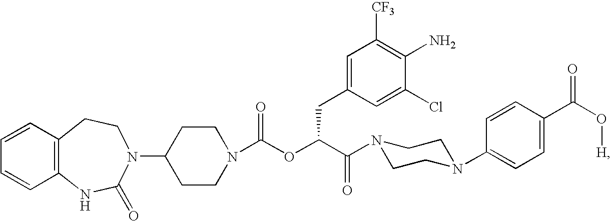 |
| (54) | 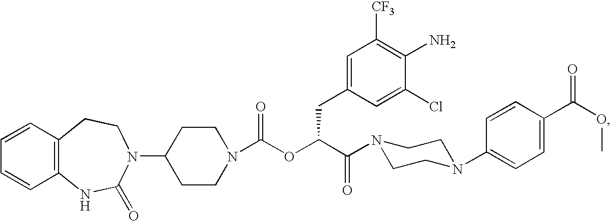 |
| (55) | 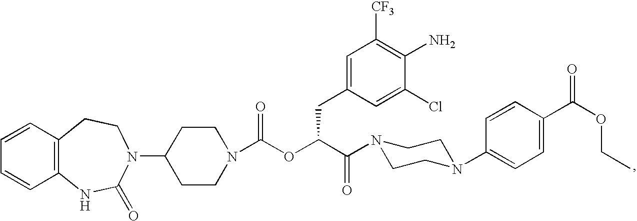 |
| (56) | 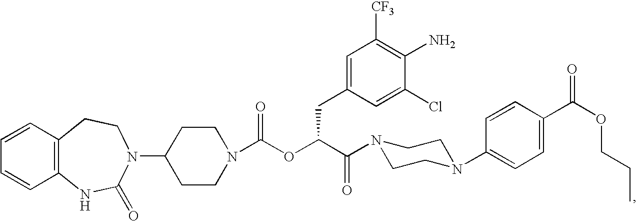 |
| (57) | 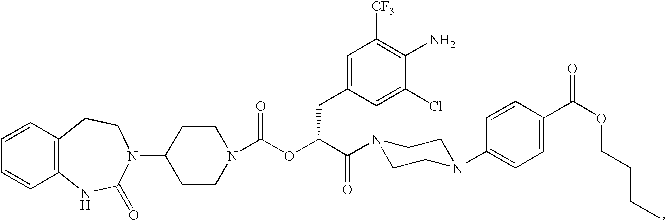 |
| (58) | 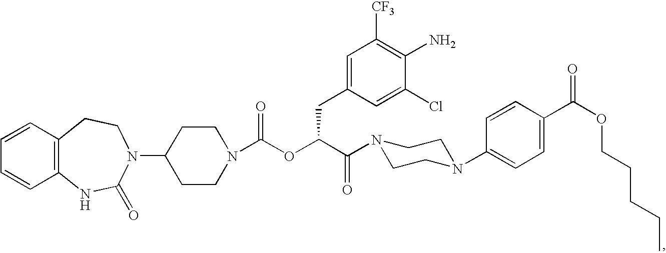 |
| (59) | 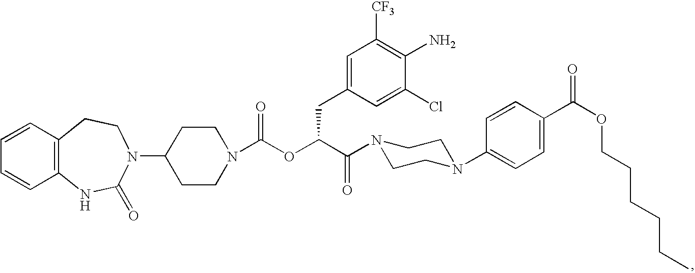 |
| (60) | 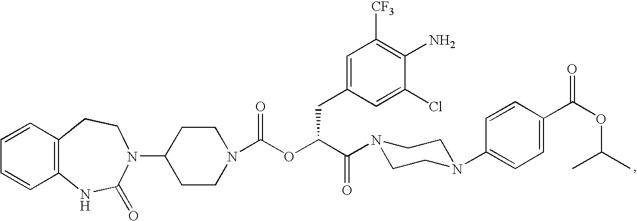 |
| (61) | 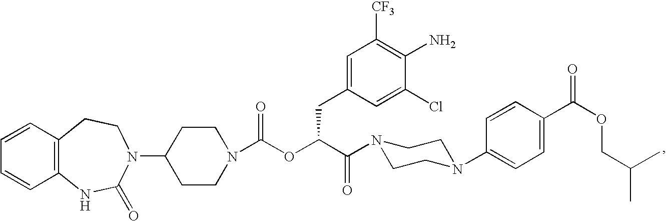 |
| (62) | 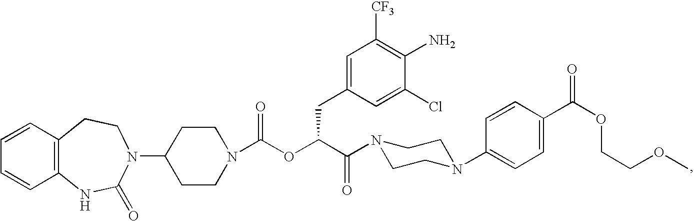 |
| (63) | 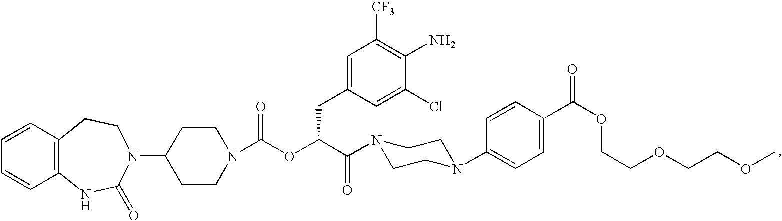 |
| (64) | 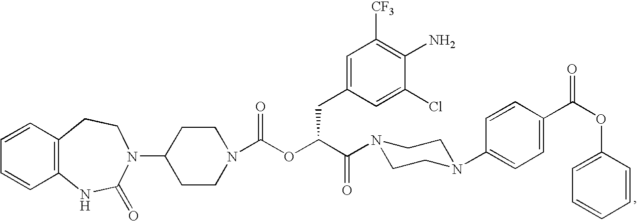 |
| (65) | 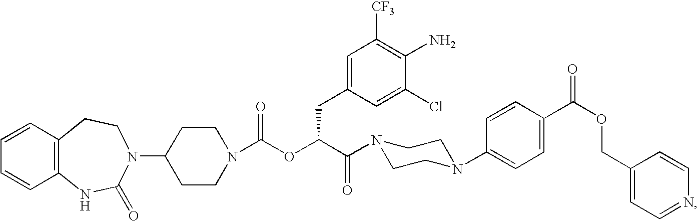 |
| (66) | 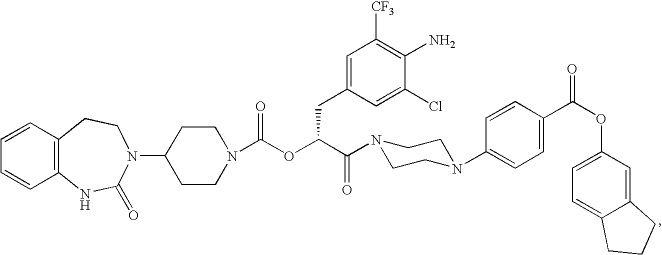 |
| (67) | 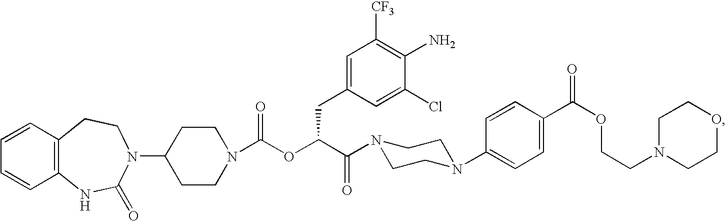 |
| (68) | 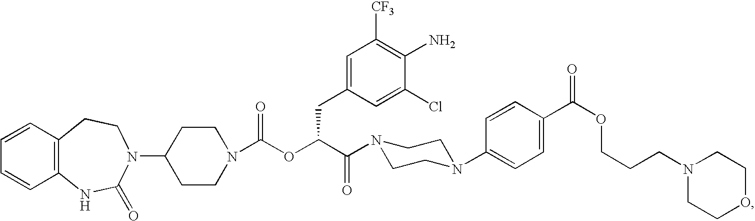 |
| (69) | 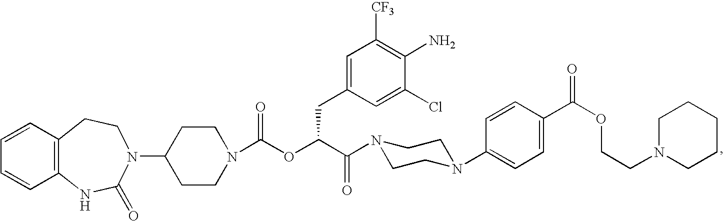 |
| (70) | 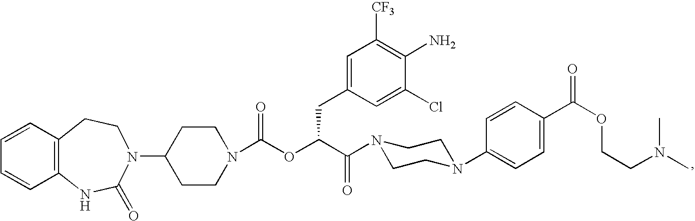 |
| (71) | 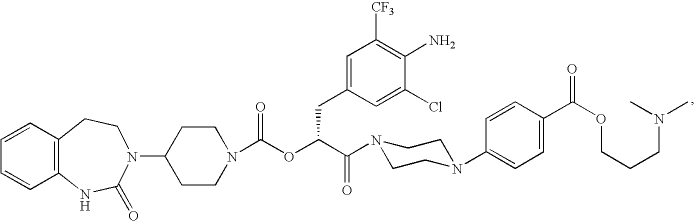 |
| (72) | 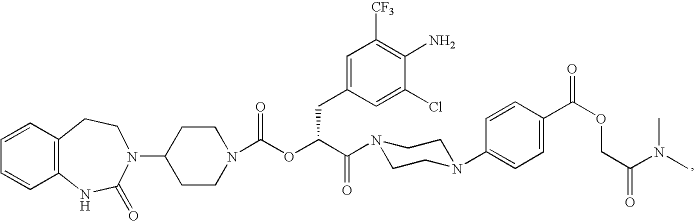 |
| (73) | 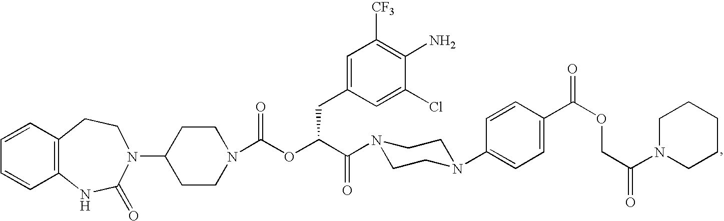 |
| (74) | 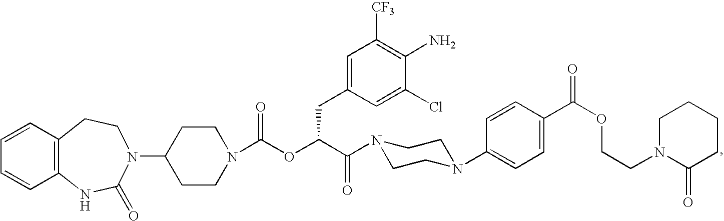 |
| (75) | 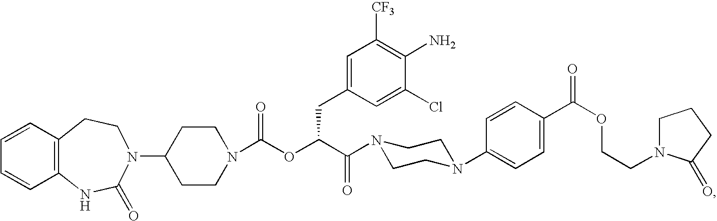 |
| (76) | 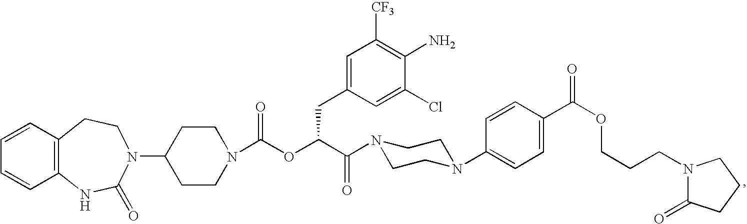 |
| (77) | 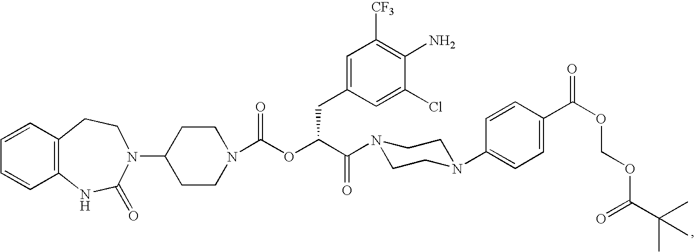 |
| (78) | 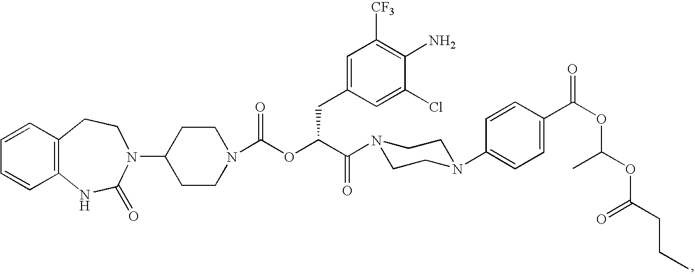 |
| (79) | 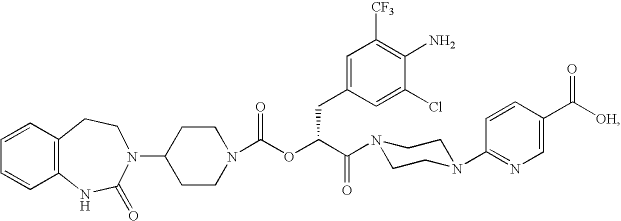 |
| (80) | 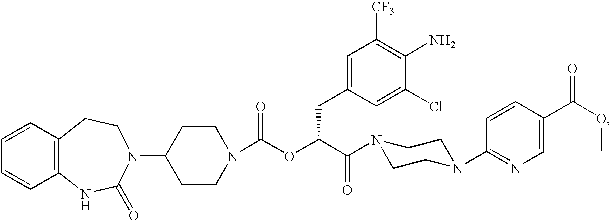 |
| (81) | 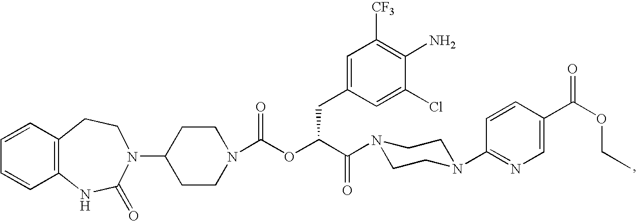 |
| (82) | 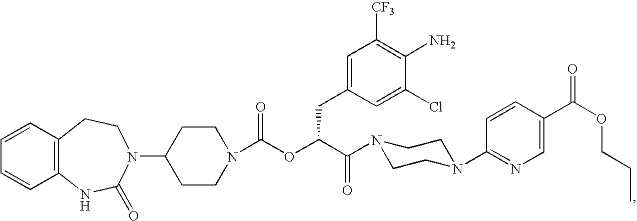 |
| (83) | 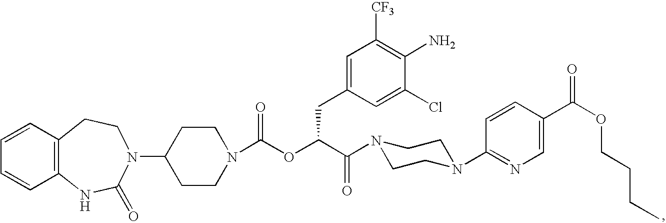 |
| (84) | 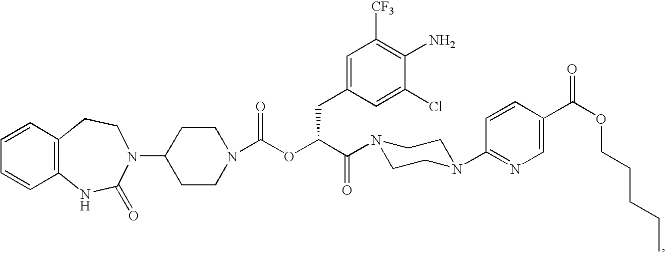 |
| (85) | 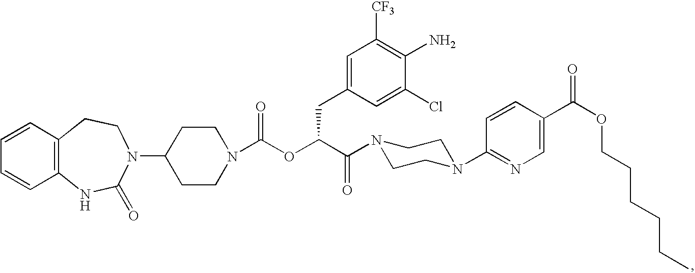 |
| (86) | 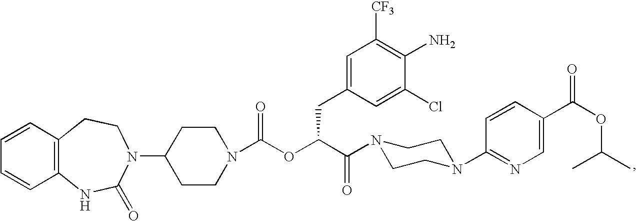 |
| (87) | 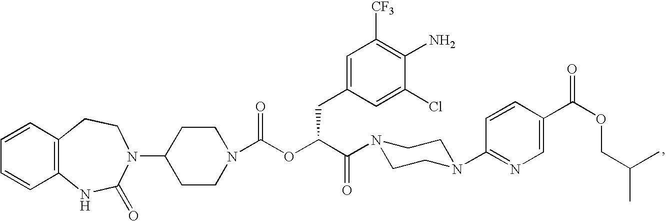 |
| (88) | 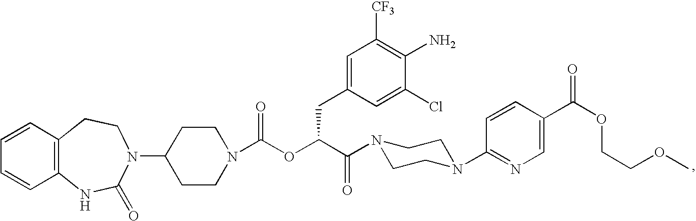 |
| (89) | 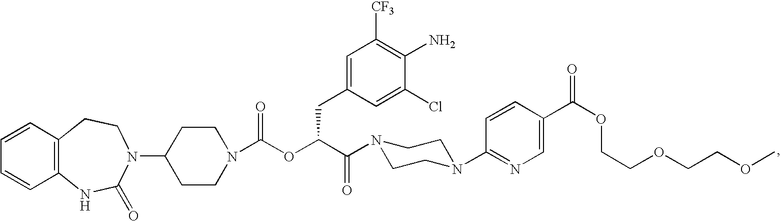 |
| (90) | 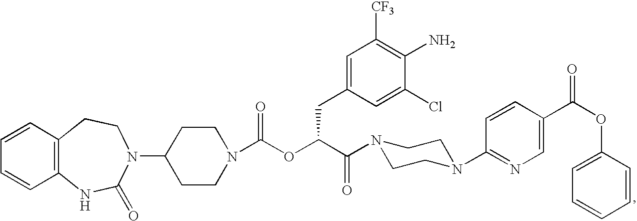 |
| (91) | 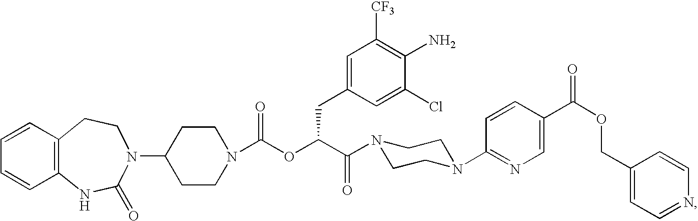 |
| (92) | 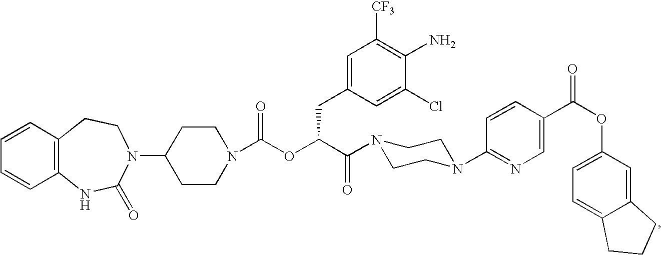 |
| (93) | 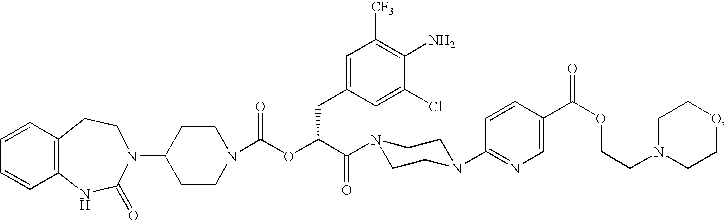 |
| (94) | 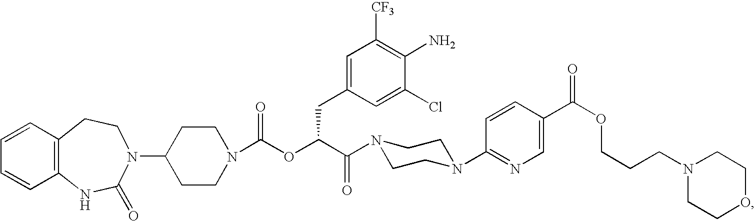 |
| (95) | 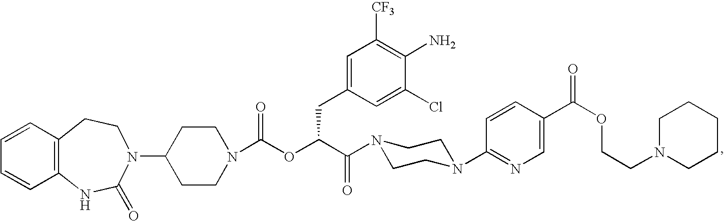 |
| (96) | 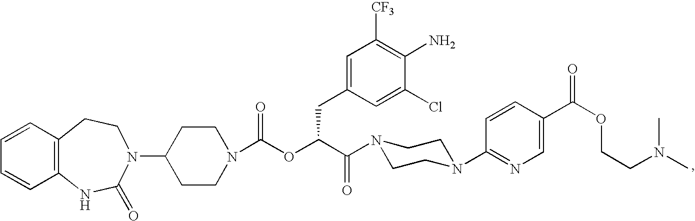 |
| (97) | 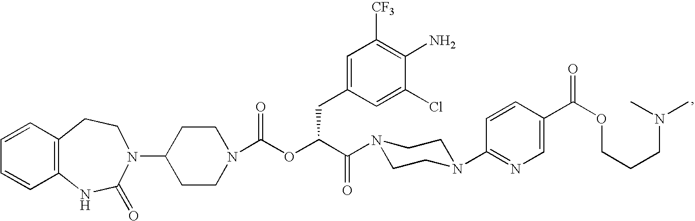 |
| (98) | 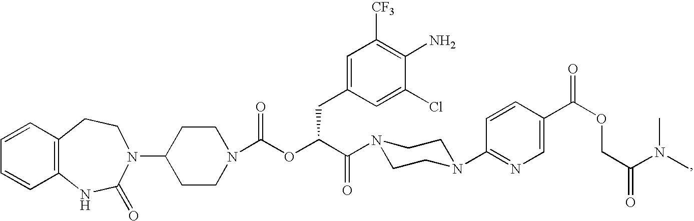 |
| (99) | 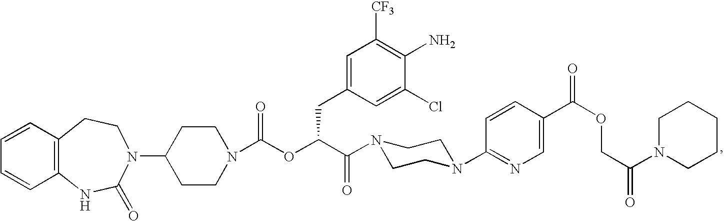 |
| (100) | 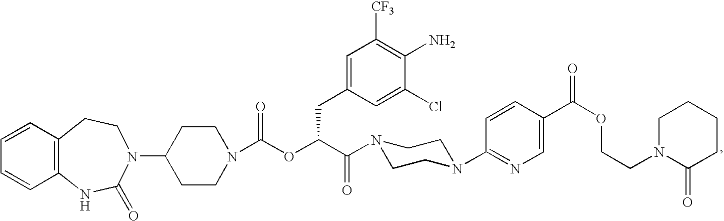 |
| (101) | 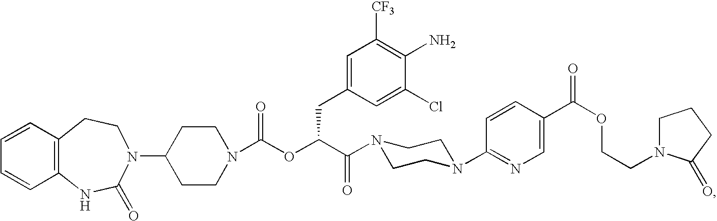 |
| (102) | 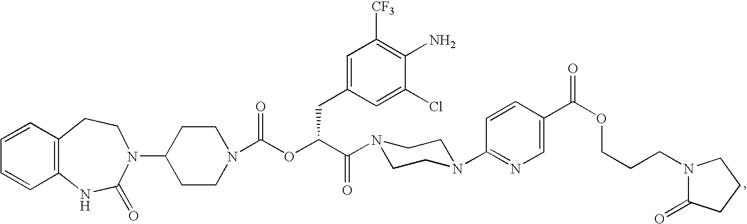 |
| (103) | 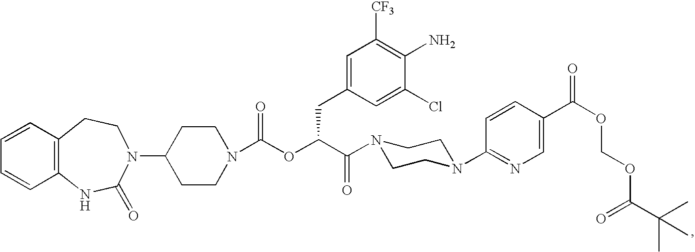 |
| (104) | 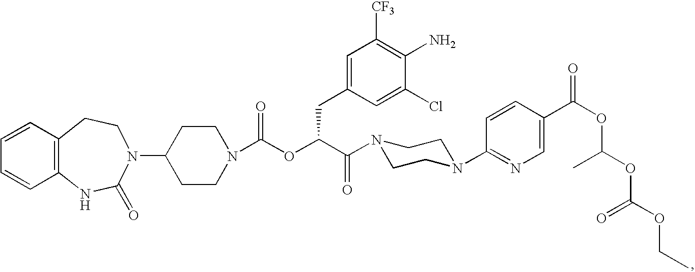 |
| (105) | 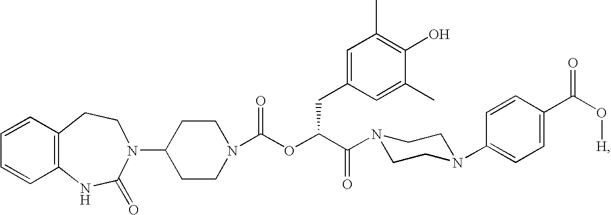 |
| (106) | 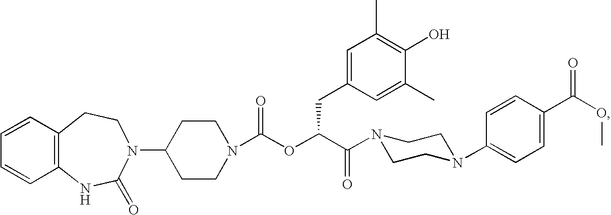 |
| (107) | 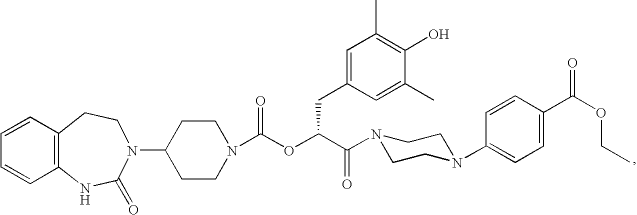 |
| (108) | 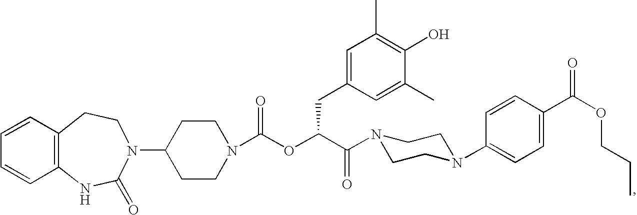 |
| (109) | 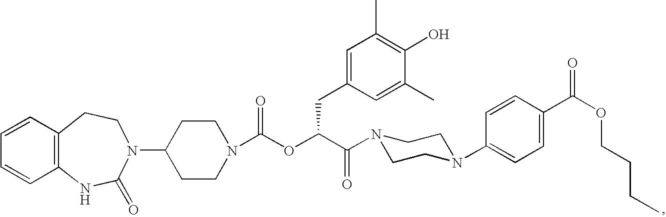 |
| (110) | 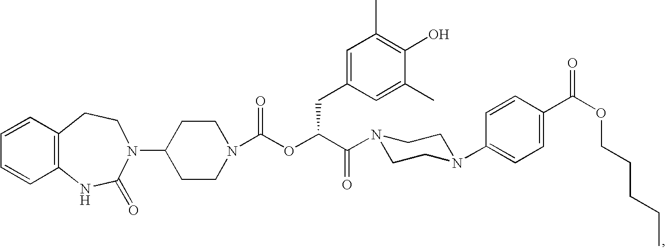 |
| (111) | 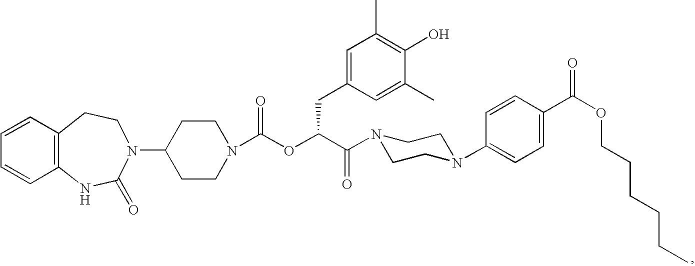 |
| (112) | 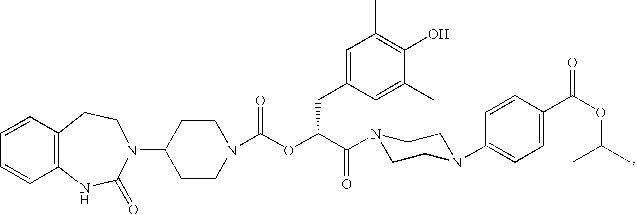 |
| (113) | 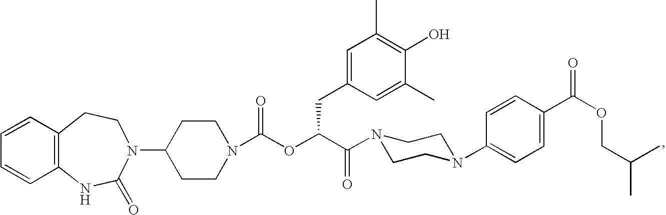 |
| (114) | 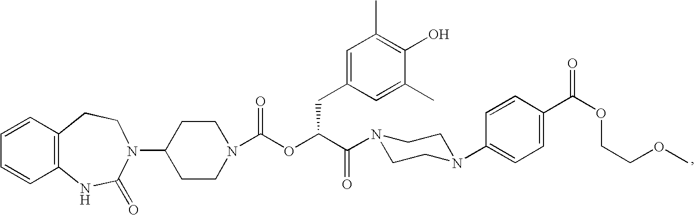 |
| (115) | 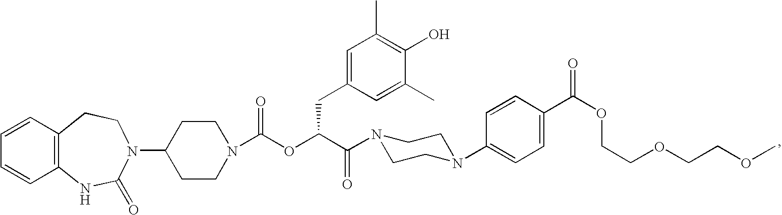 |
| (116) | 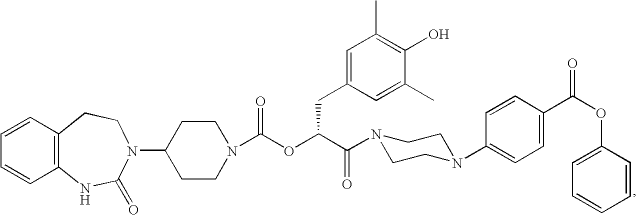 |
| (117) | 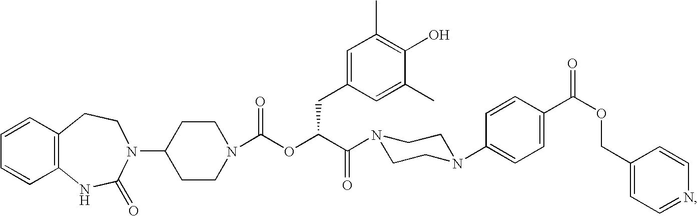 |
| (118) | 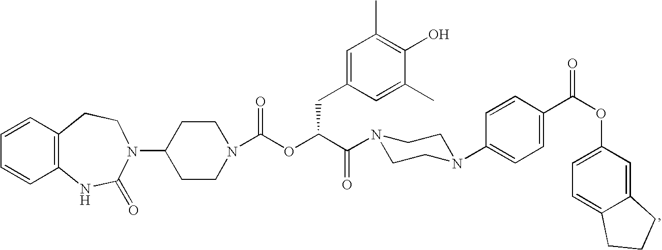 |
| (119) | 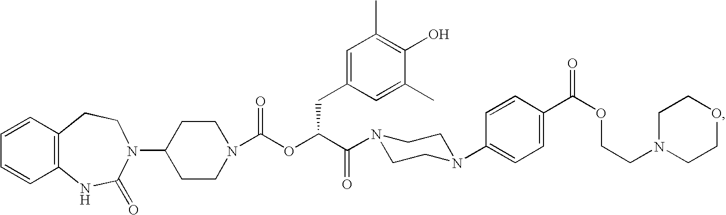 |
| (120) | 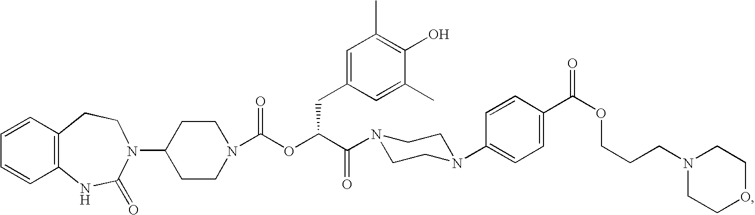 |
| (121) | 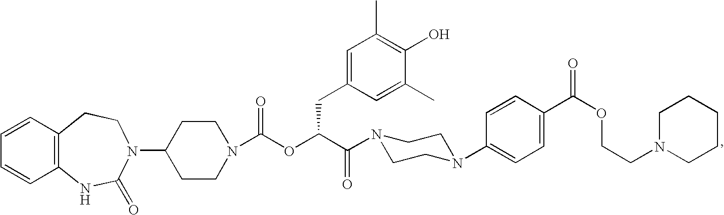 |
| (122) | 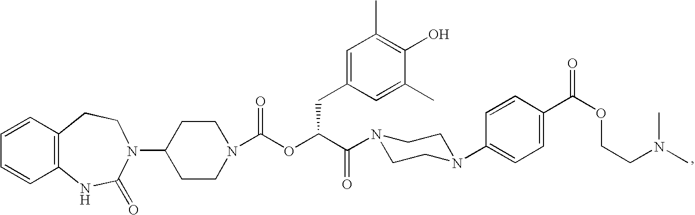 |
| (123) | 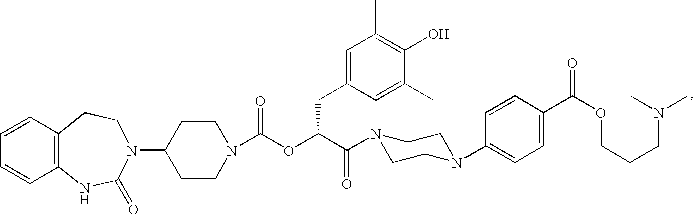 |
| (124) | 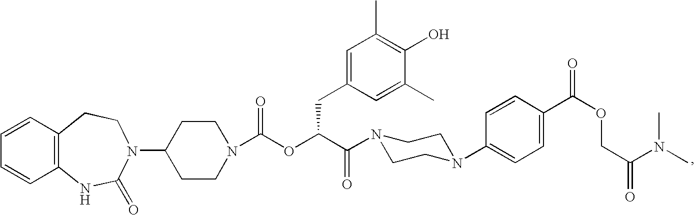 |
| (125) | 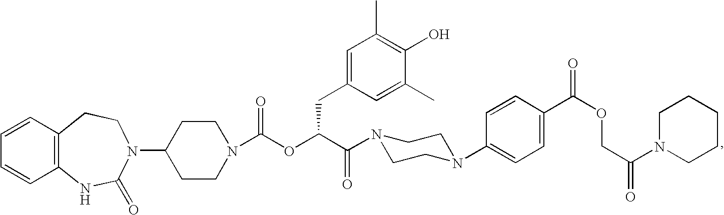 |
| (126) | 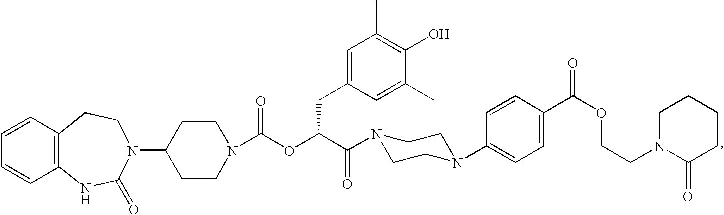 |
| (127) | 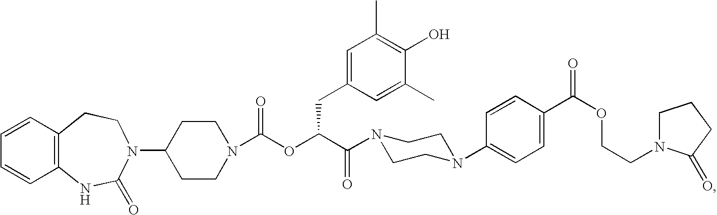 |
| (128) | 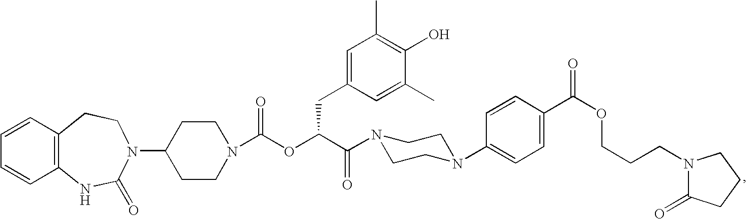 |
| (129) | 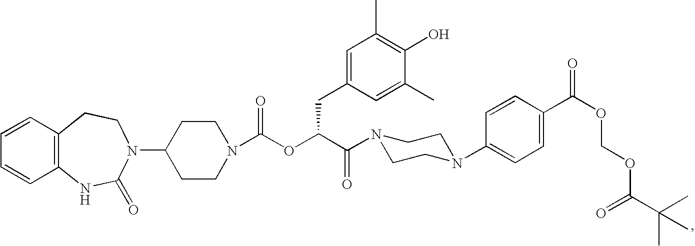 |
| (130) | 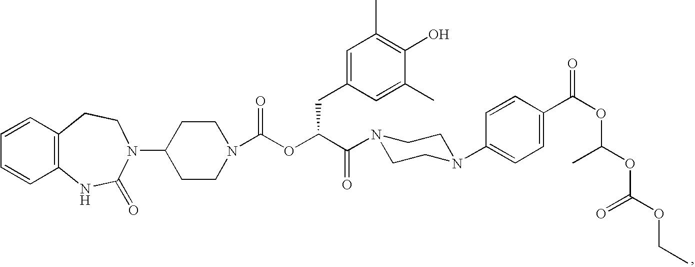 |
| (131) | 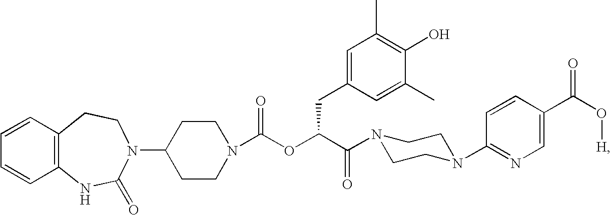 |
| (132) | 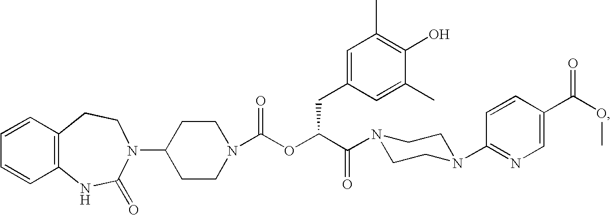 |
| (133) | 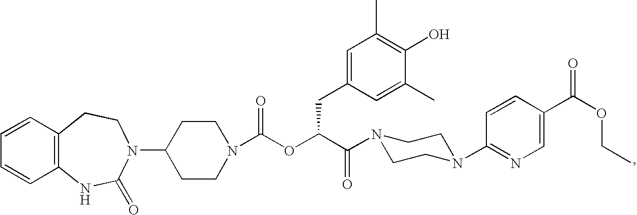 |
| (134) | 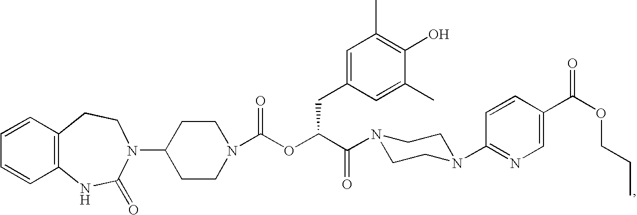 |
| (135) | 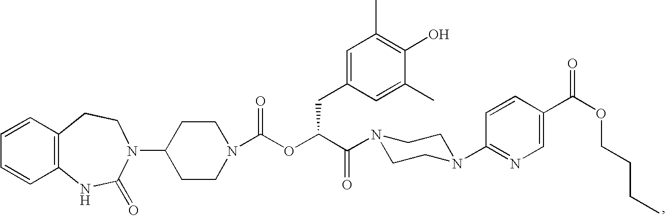 |
| (136) | 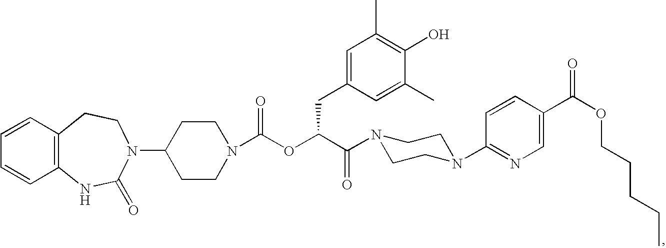 |
| (137) | 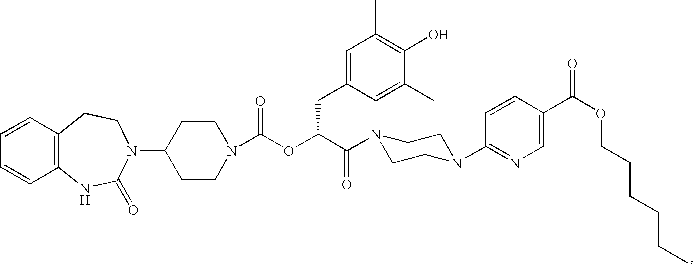 |
| (138) | 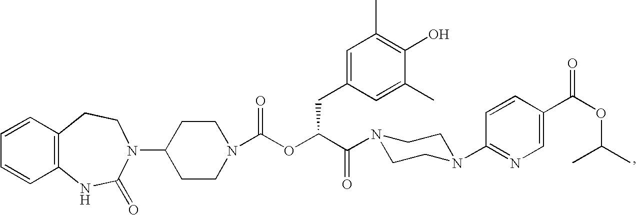 |
| (139) | 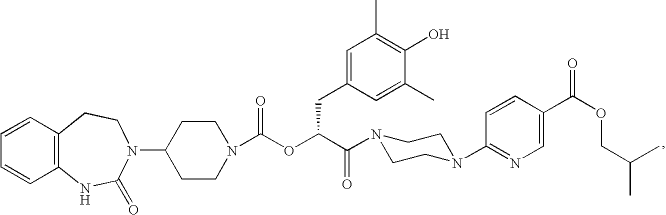 |
| (140) | 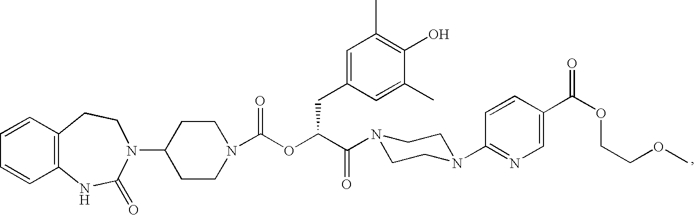 |
| (141) | 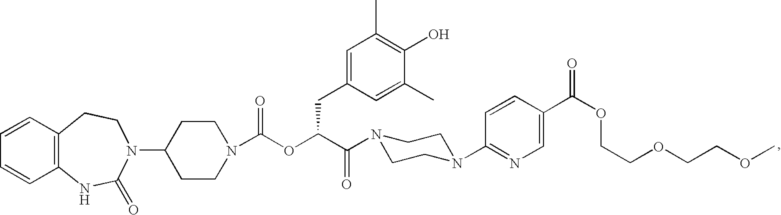 |
| (142) | 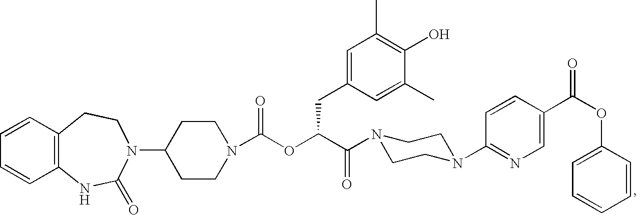 |
| (143) | 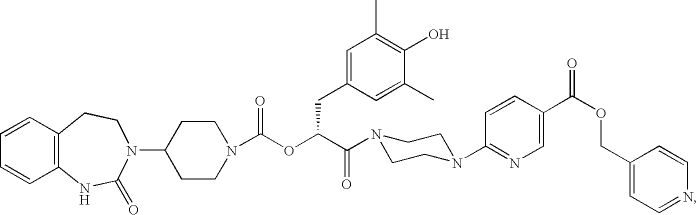 |
| (144) | 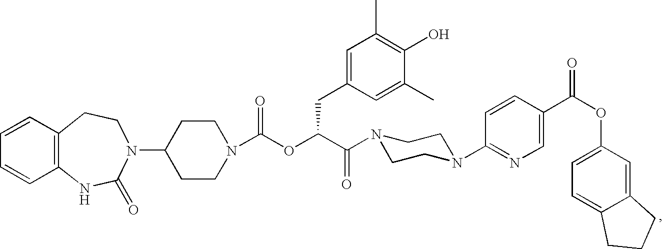 |
| (145) | 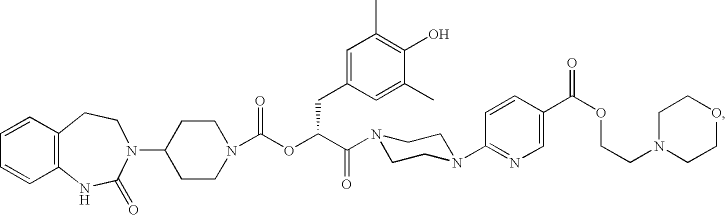 |
| (146) | 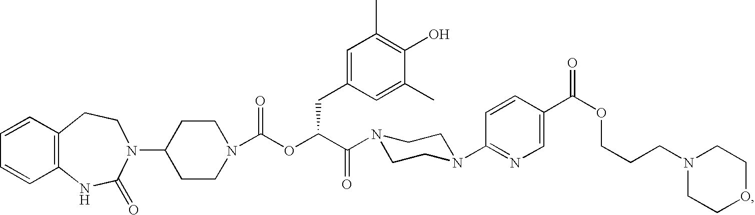 |
| (147) | 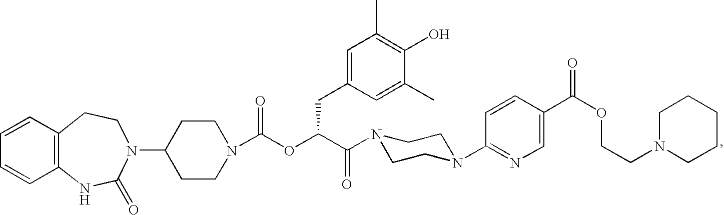 |
| (148) | 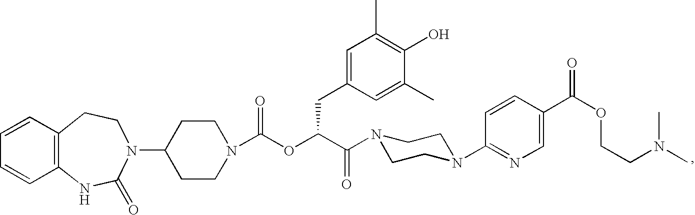 |
| (149) | 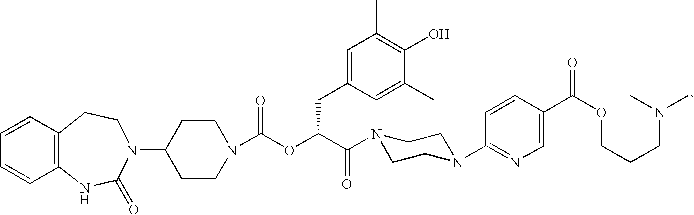 |
| (150) | 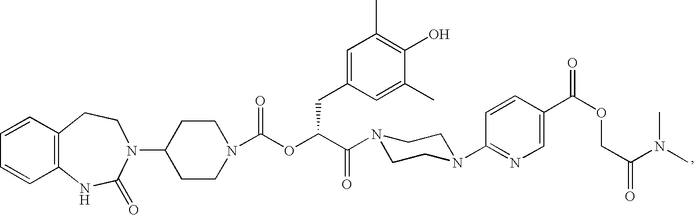 |
| (151) | 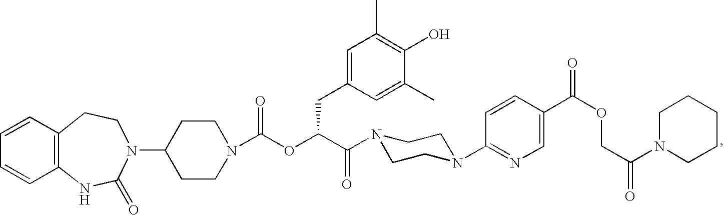 |
| (152) | 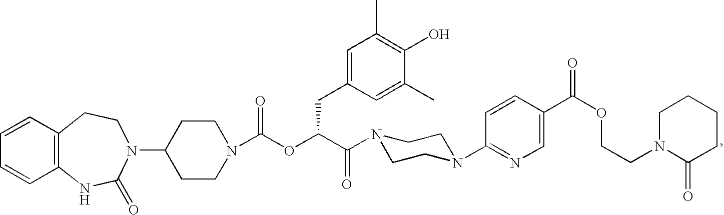 |
| (153) | 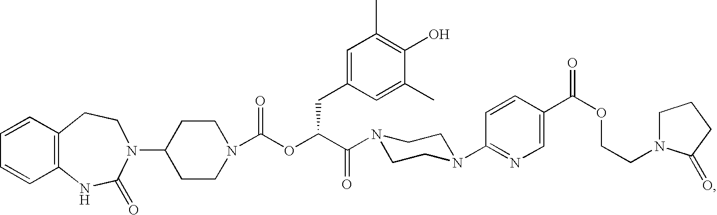 |
| (154) | 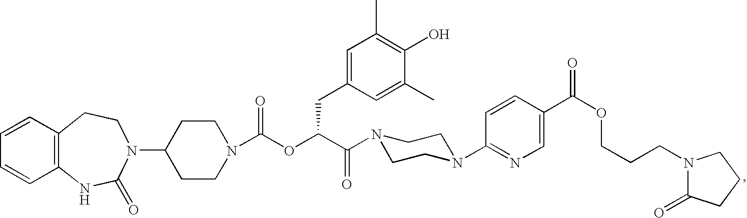 |
| (155) | 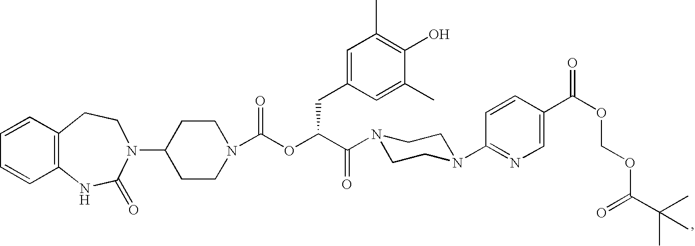 |
| (156) | 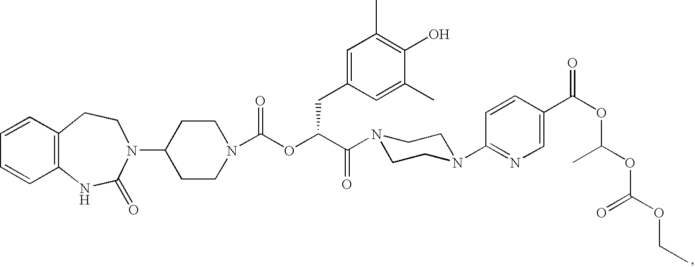 |
| (157) | 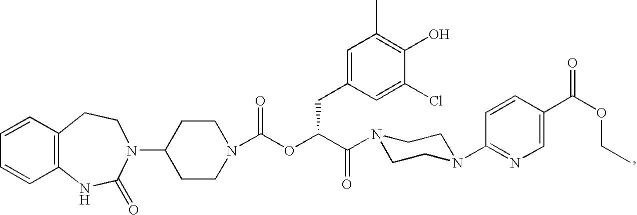 |
| (158) | 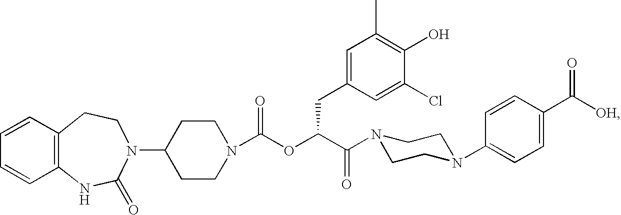 |
| (159) | 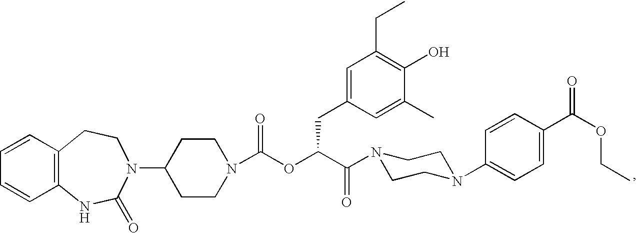 |
| (160) | 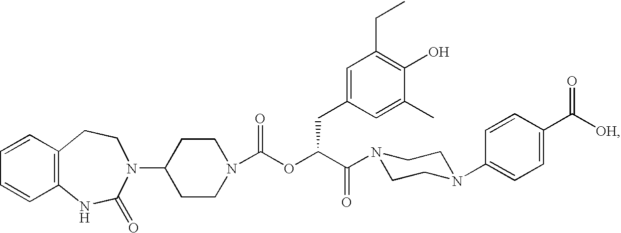 |
| (161) | 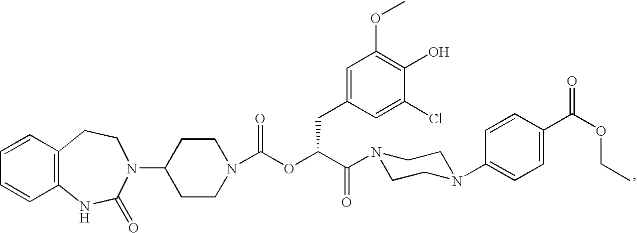 |
| (162) | 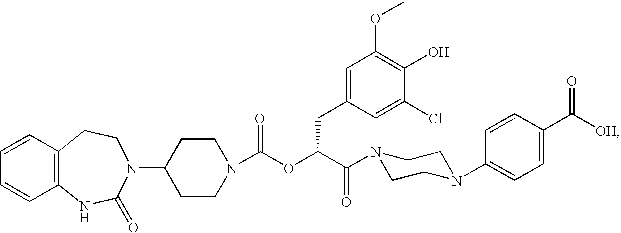 |
| (163) | 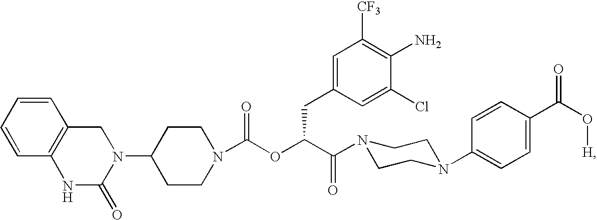 |
| (164) | 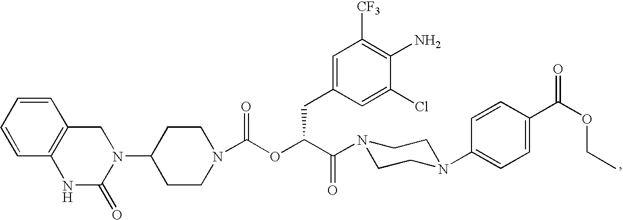 |
| (165) | 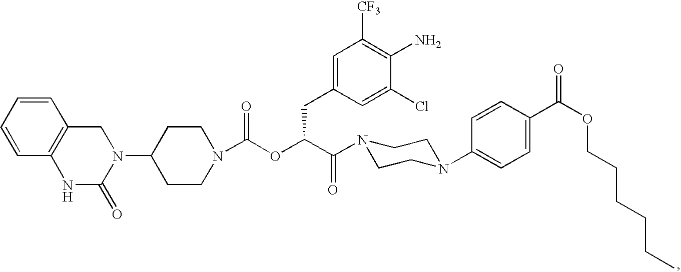 |
| (166) | 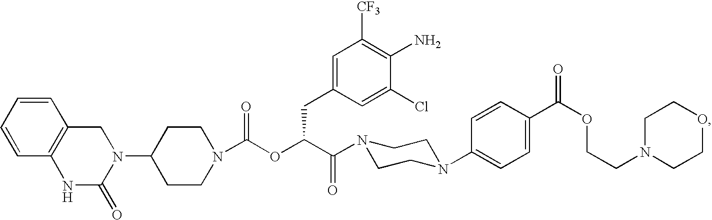 |
| (167) | 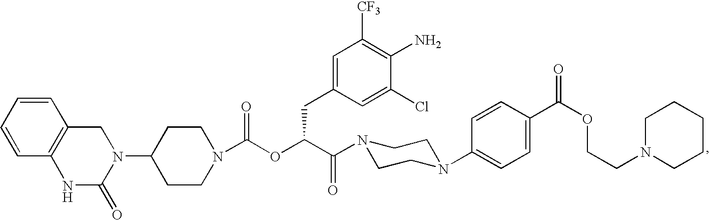 |
| (168) | 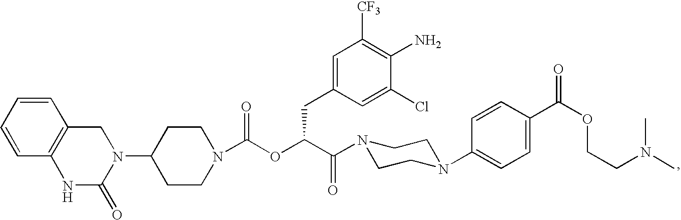 |
| (169) | 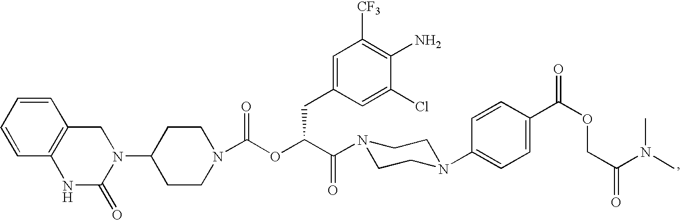 |
| (170) | 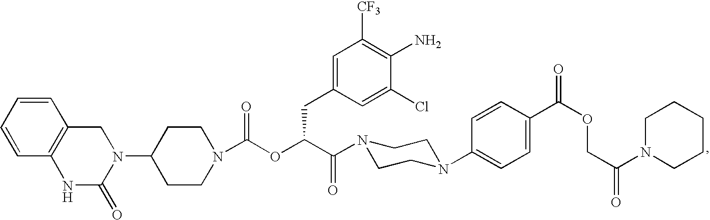 |
| (171) | 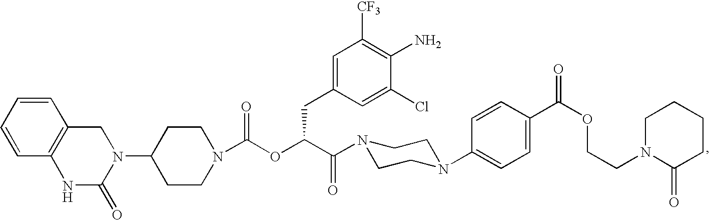 |
| (172) | 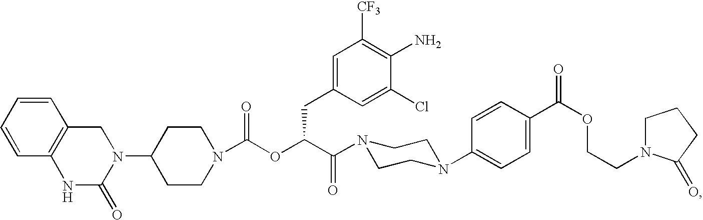 |
| (173) | 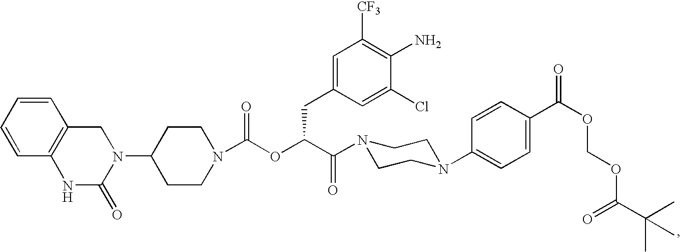 |
| (174) | 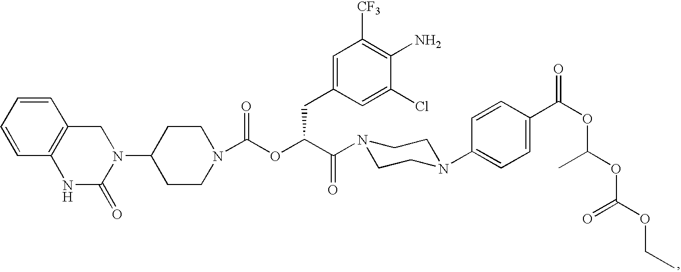 |
| (175) | 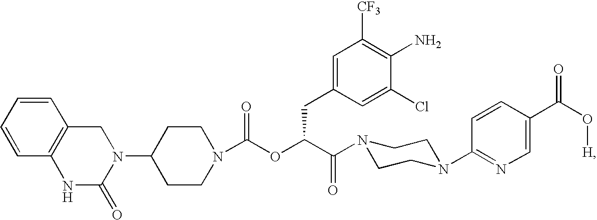 |
| (176) | 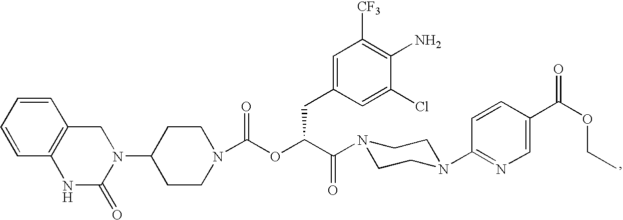 |
| (177) | 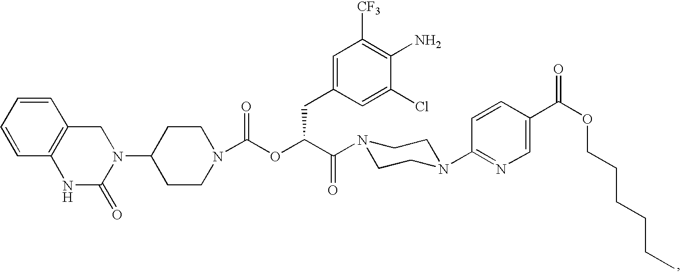 |
| (178) | 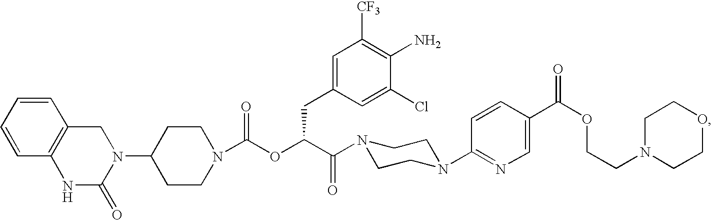 |
| (179) | 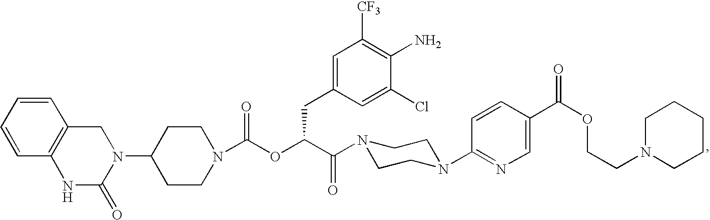 |
| (180) | 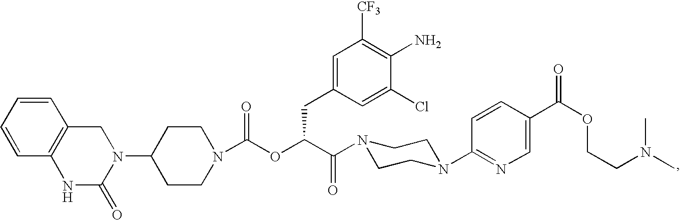 |
| (181) | 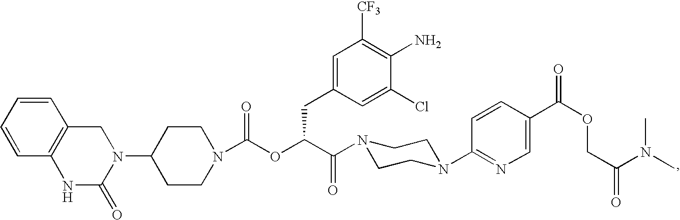 |
| (182) | 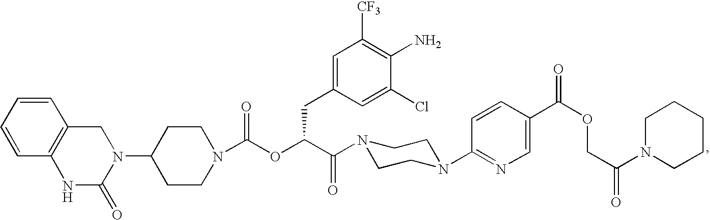 |
| (183) | 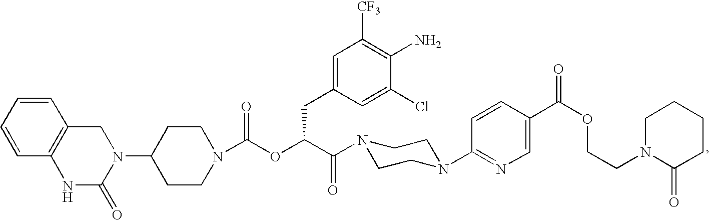 |
| (184) | 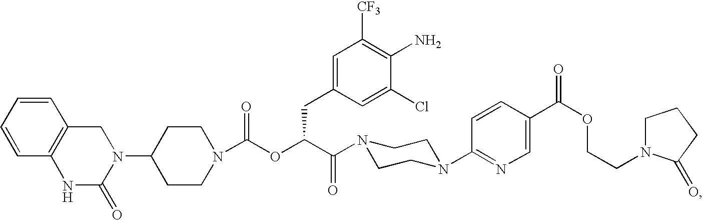 |
| (185) | 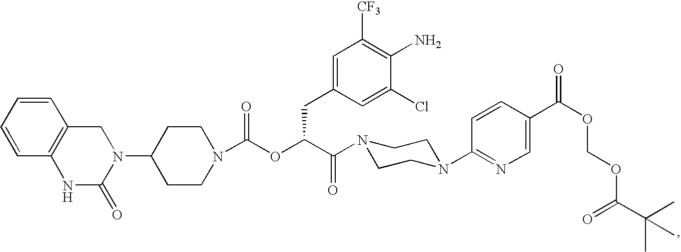 |
| (186) | 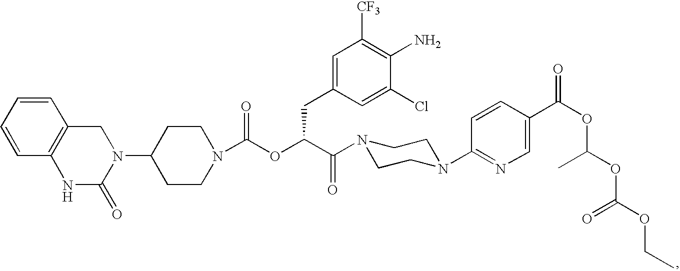 |
| (187) | 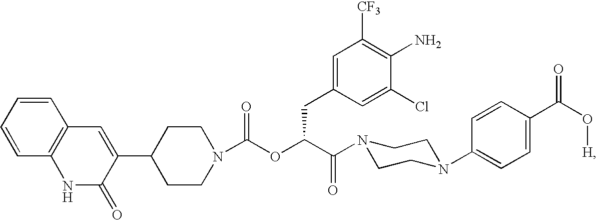 |
| (188) | 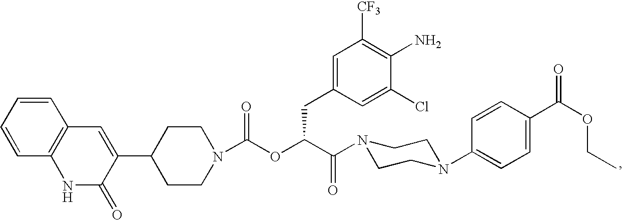 |
| (189) | 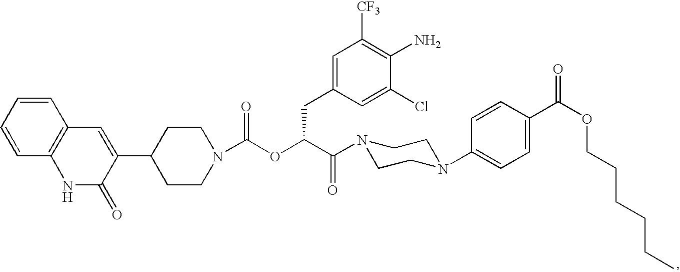 |
| (190) | 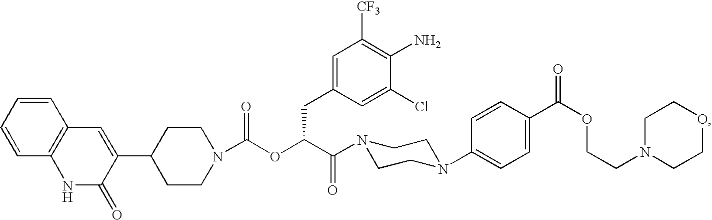 |
| (191) | 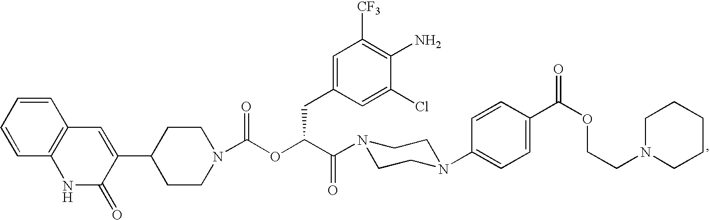 |
| (192) | 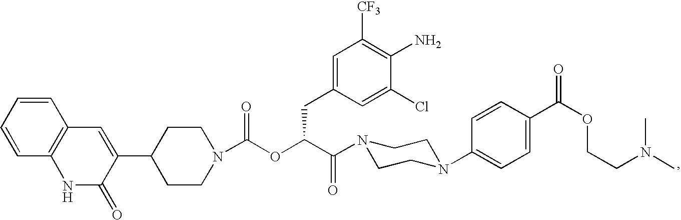 |
| (193) | 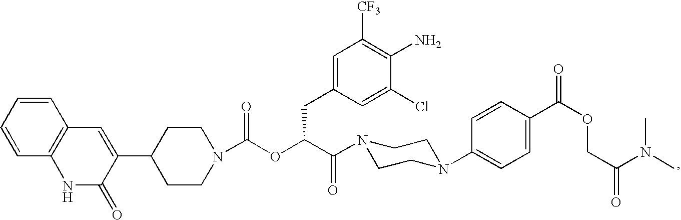 |
| (194) | 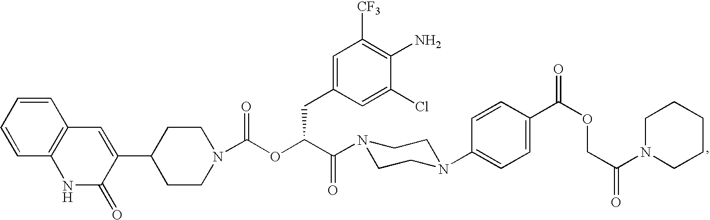 |
| (195) | 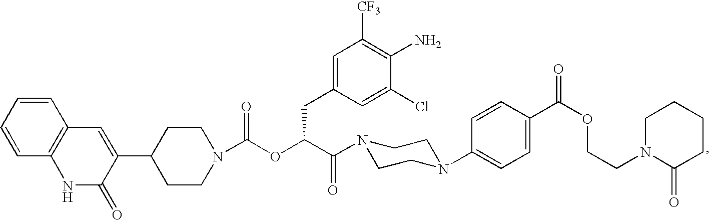 |
| (196) | 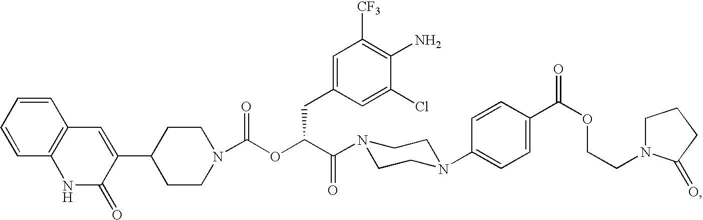 |
| (197) | 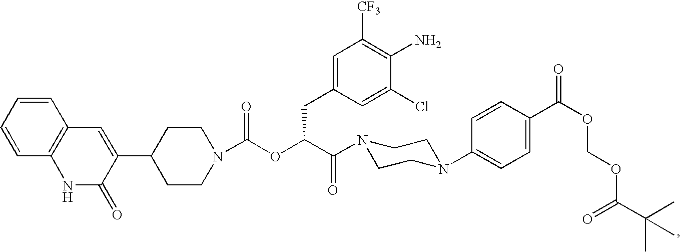 |
| (198) | 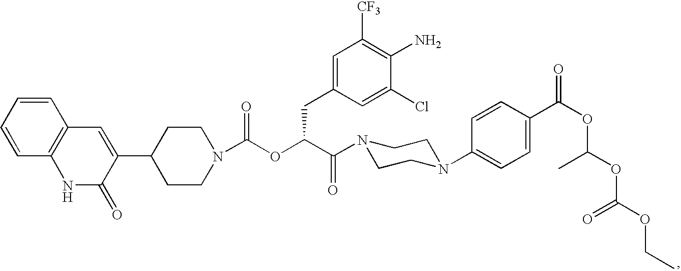 |
| (199) | 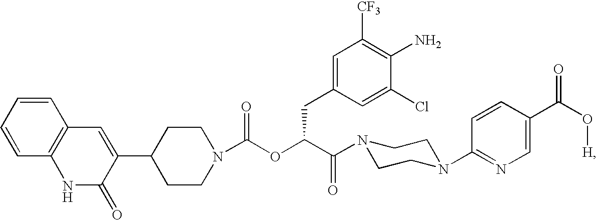 |
| (200) | 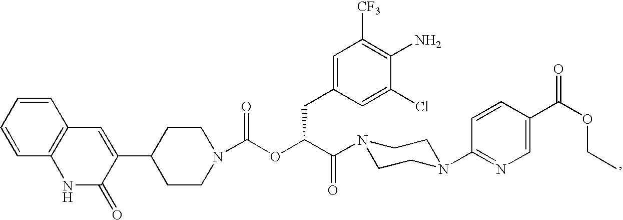 |
| (201) | 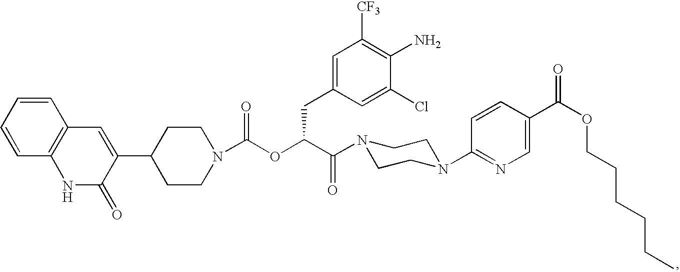 |
| (202) | 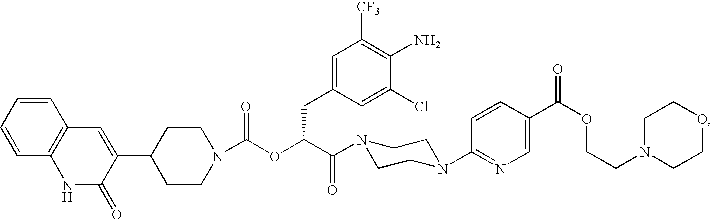 |
| (203) | 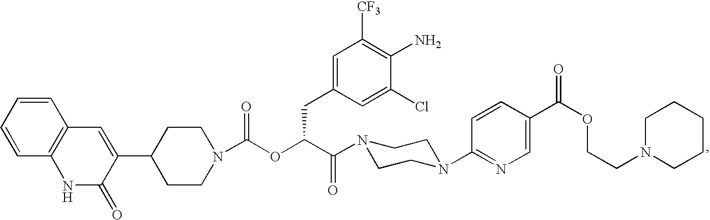 |
| (204) | 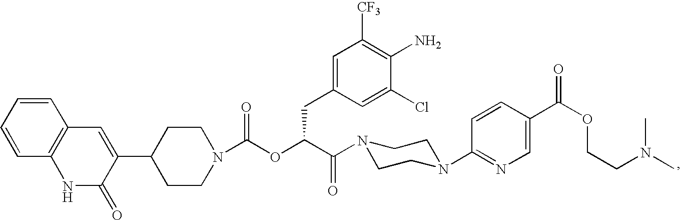 |
| (205) | 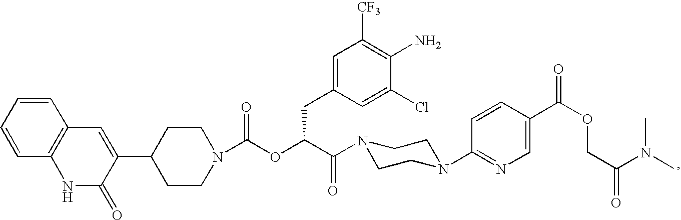 |
| (206) | 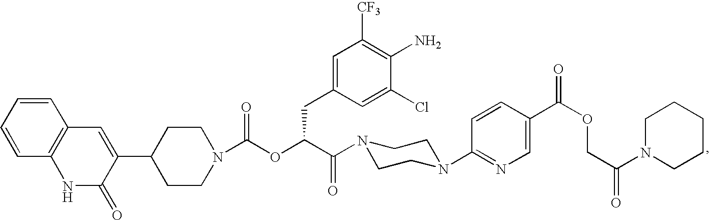 |
| (207) | 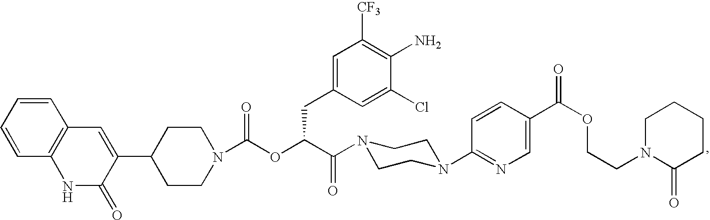 |
| (208) | 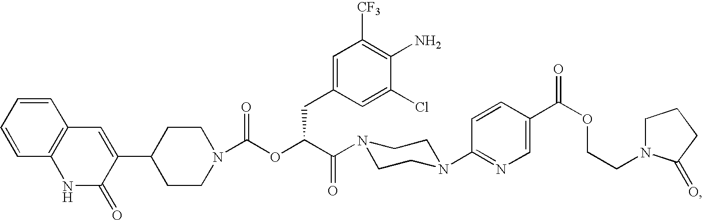 |
| (209) | 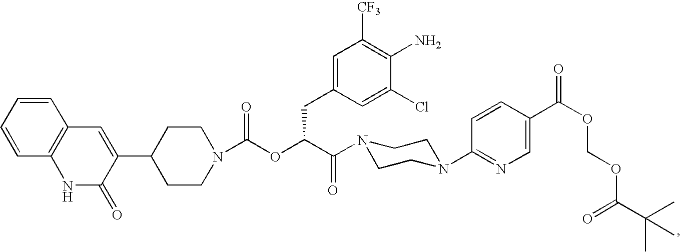 |
| (210) | 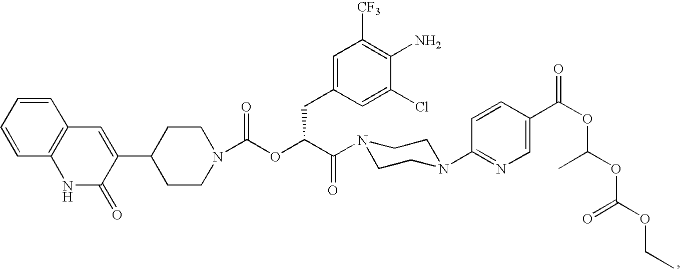 |
| (211) | 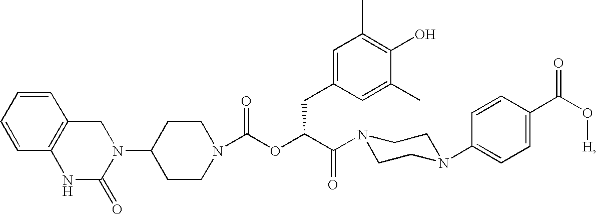 |
| (212) | 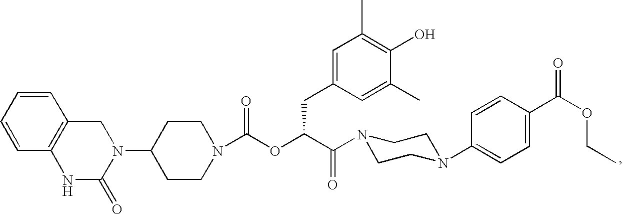 |
| (213) | 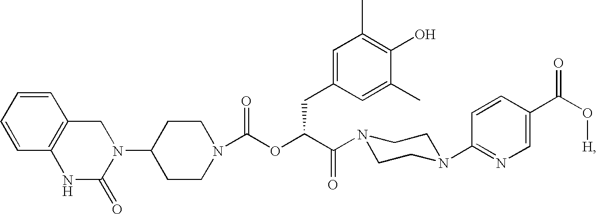 |
| (214) | 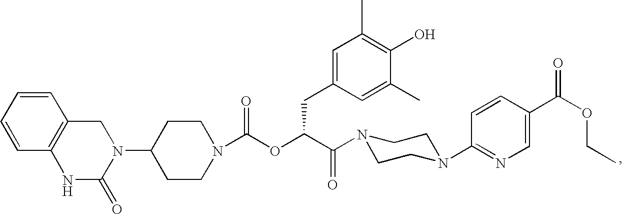 |
| (215) | 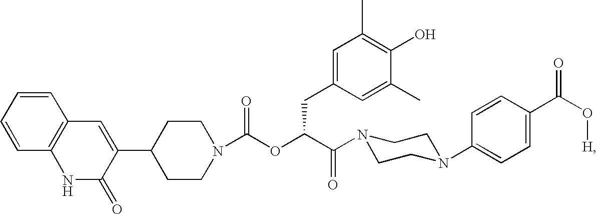 |
| (216) | 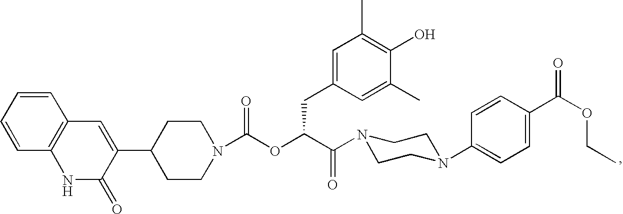 |
| (217) | 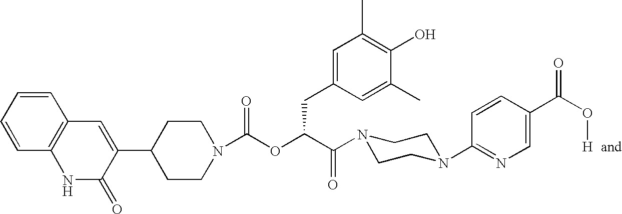 |
| (218) | 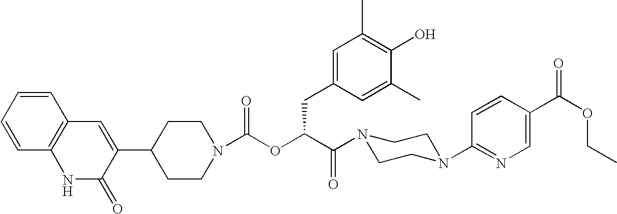 |

or a tautomer or salt thereof.

5. A physiologically acceptable salt of a compound according to claim 1, 2, 3 or 4.

6. A pharmaceutical composition comprising a compound according to claim 1, 2, 3 or 4 or a physiologically acceptable salt thereof, together with a pharmaceutically acceptable carrier or diluent.

7. A method for treating migraine, cluster or tension headaches, which comprises administering to a host suffering from such a headache a therapeutically effective amount of a compound according to claim 1, 2, 3 or 4 or a physiologically acceptable salt thereof.

8. A method for treating non-insulin-dependent diabetes mellitus (NIDDM), which comprises administering to a host suffering from such NIDDM, a therapeutically effective amount of a compound according to claim 1, 2, 3 or 4 or a physiologically acceptable salt thereof.

9. (canceled)

10. A method for treating irritable bowel syndrome (IBS), which comprises administering to a host suffering from such IBS, a therapeutically effective amount of a compound according to claim 1, 2, 3 or 4 or a physiologically acceptable salt thereof.

11. A method for treating hot flushes in oestrogen-deficient women, which comprises administering to anoestrogen-deficient women suffering from hot flushes, a therapeutically effective amount of a compound according to claim 1, 2, 3 or 4 or a physiologically acceptable salt thereof.

12-13. (canceled)
